(19) United States
(12) Patent Application Publication
(10) Pub. No.: US 2025/0090729 A1
(43) Pub. Date: Mar. 20, 2025

(54) Methods and Products to Detect, Minimize and Treat TRAP-Related Tissue Reactions and Tissue Injury Associated With Medical Devices

(71) Applicant: Cell and Molecular Tissue Engineering, LLC, Avon, CT (US)

(72) Inventors: Donald L. Kreutzer, Avon, CT (US); Ulrike W. Klueh, Detroit, MI (US)

(73) Assignee: Cell and Molecular Tissue Engineering, LLC, Avon, CT (US)

(21) Appl. No.: 18/729,359

(22) PCT Filed: Jan. 19, 2023
(86) PCT No.: PCT/US2023/011186

§ 371 Date: Jul. 16, 2024

Related U.S. Application Data
(60) Provisional application No. 63/301,397, filed on Jan. 20, 2022.

(51) Int. Cl. A61L 29/08; A61L 29/16

(52) U.S. Cl. CPC A61L 29/08; A61L 29/16; A61L 2300/41; A61L 2300/434; A61L 2300/61; A61L 2300/626

(57) ABSTRACT

Disclosed herein are implantable and non-implantable devices coated with an anti-TRAP agent. A system is also disclosed comprising an implantable device or a non-implantable device and a tissue-injectable/infusible liquid, oil or gel comprising an anti-TRAP agent. A system incorporating a drug-injecting pen is described. Medical biomaterials that inhibit TRAPs are discussed, as well as air pouch models for analyzing products and materials. Corresponding methods also are described.

### RELATED APPLICATIONS

This application claims the benefit of U.S. Patent Application No. 63/301,397 filed Jan. 20, 2023, the contents of which are incorporated by reference herein in their entirety.

### STATEMENT WITH REGARD TO FEDERALLY SPONSORED RESEARCH & DEVELOPMENT

Some of the embodiments described herein have been made with Government support under Grants awarded by the National Institute of Health. The Government may have certain rights in the described embodiments.

### BACKGROUND

The explosion of implantable medical devices (IMD) and medical biomaterials and medical bio-fluids, has substantially improved the quality and length of life for a wide variety of patients worldwide. In the case of implantable medical devices, these devices range from simple devices such as metal screws, surgical meshes, cannulas, shunts, or stents. Additionally, treatments with injectable or infusible medical bio-fluids (e.g., insulin) help millions of patients with diabetes maintain euglycemia. Additionally, various combinations of implantable medical devices (IMD) and medical biomaterials and medical bio-fluids can be integrated in composite devices, such as implantable medical devices (TMD), medical biomaterials and medical-bio-fluids, e.g., the artificial pancreas. In the case of the artificial pancreas, it is a combination of implantable medical devices (sensors and cannulas) and medical bio-fluids (i.e., insulin). All these medical devices are dependent on bio-compatible medical biomaterials and medical bio-fluids, to function successfully in vivo.

Proper functioning of medical devices, medical bio-fluids and medical bio-materials are critical to the treatment of human and animal diseases both now and in the future. Success in these devices, materials and biofluids depends on their biocompatibility with biologic fluids, cells, tissues and organs, in vitro, in vivo, ex vivo and or extracorporeally.

Blood, cells and tissues, directly in contact in vitro applications, in vivo, ex vivo or extracorporeally, in conjunction with medical devices, medical bio-materials, medical bio-fluids, can result in triggering destructive biologic fluid activation (e.g. blood clotting, complement activation), cell injury, cell activation and cell death, resulting in tissue reactions (e.g. inflammation, loss of blood vessels and fibrosis), that limit the performance of these medical devices, medical bio-fluids and/or medical biomaterials; e.g., implantable sensors, insulin infusion/injections to maintain blood glucose levels (euglycemia) in patients with diabetes, stent and surgical mesh, as well as heart and kidney bypass related medical devices. Thus, the functional lifespans of these medical devices, medical biofluids and biomaterials, as well as related treatments, are frequently shortened or hindered by tissue reactions triggered by these medical biofluids, biomaterials, devices and treatments.

Although there have been decades of research and development to discover a “Rosetta Stone” to defeat tissue reactions to medical devices, medical biofluids and or medical biomaterials and treatments, no true breakthroughs or disruptive technology has been developed to overcome these tissue reactions, and thereby extend the functional accuracy and lifespan of implantable devices in vitro, in vivo, extracorporeally or ex vivo. Thus, there is a clear need for the advancement of a truly disruptive technology to fill this critical need for better functioning and longer lasting medical devices, medical bio-fluids medical bio-materials and related treatments. Additionally, there is poor understanding of the “nature” of the materials and fluids that are used to build and use these medical devices, medical bio-fluids and medical bio-materials as well as associated treatments, and their role in triggering and or sustaining destructive tissue reactions associated with these devices, fluids and treatments. This includes developing in vitro and in vivo assays (e.g. biomarkers) that predict the likelihood that a medical bio-fluids, biomaterials, medical devices and related treatments trigger destructive cell and tissue reactions that limit the lifespan and or functions of medical bio-fluids, biomaterials, medical devices and related treatments in vitro, in vivo, extracorporeally or ex vivo.

Despite their demonstrated clinical benefits, currently, insulin infusion sets are only approved for in vivo usage for only a few days. Even with this limited approved lifespan, a substantial portion of sets fail to meet this recommended lifespan during practical use.

Nevertheless, Continuous Subcutaneous Insulin Infusion (CSII) therapy represents the most advanced form of insulin delivery technology currently available and administers more precise amounts of insulin in a programmable format as compared to traditional injection methods, which provides increased flexibility and enhanced quality of life for the user. To achieve effective glucose stasis using an artificial pancreas, a combination of a highly accurate continuous glucose monitor (CGM) and reliable continuous subcutaneous insulin infusion (CSII) is required. Although CGM performance and lifespan has significantly improved over the last decade, CSII with a current lifespan of 3 days or less has not. As such, the currently approved usage lifespans for commercial CGM and CSII devices are highly mismatched with in vivo durations of 10+ days vs. 3 days, respectively. Other medical devices and biofluids also have limited useful lifespans.

A body's response to the presence of a foreign substances, whether microbes e.g., bacteria or viruses) or their products (e.g. toxins (e.g. endotoxins), or non-infective/non-microbial materials or substances e.g., sterile foreign objects like medical devices, medical bio-materials, and medical bio-fluids), triggers non-specific and/or specific activation of the immune system (i.e. innate and acquired immunity). Generally, these reactions begin with the innate immune system followed by the adaptive immune system. Both innate and acquired immunity involve biologic fluids (e.g., blood, plasma serum), cells (white blood cells) and tissue cells (e.g., endothelial cells and fibroblasts), proteins (e.g. fibril clots, complements proteins, or antibodies) usually resulting in cell injury and death as well as tissue destruction involving inflammation, fibrosis and loss of vascular vessels, resulting loss of tissue architecture and function.

For the effective treatment of specific human and animal diseases, e.g. treatment utilizing medical devices, medical bio-fluids and medical bio-materials, it is important to develop products and methods to reduce tissue inflammation and scarring, and loss of blood vessels resulting from contact between non-microbial foreign substances, e.g., medical devices, medical bio-fluids, and medical bio-materials, and body cells, tissues, and fluids including blood and blood components, as well as cells in the skin layers and muscle tissues of the body.

### SUMMARY

One embodiment described herein is an implantable device coated with an anti-TRAP agent.

Another embodiment is a method comprising obtaining an anti-TRAP agent, obtaining an implantable device, coating the implantable device with the anti-TRAP agent, and implanting the device.

Another embodiment is a non-implantable medical device coated with an anti-TRAP agent.

A further embodiment is a method comprising obtaining an anti-TRAP agent, obtaining a non-implantable device, coating the non-implantable device with the anti-TRAP agent, and using the device.

A further embodiment is a system comprising an implantable device or a non-implantable device and a tissue-injectable and/or infusible liquid, oil or gel comprising an anti-TRAP agent.

Yet another embodiment is a method of treating a tissue containing an implantable device, comprising delivering to the tissue a therapeutically effective quantity of an anti-TRAP agent.

Another embodiment is a method of increasing the lifespan of an implantable device comprising delivering a therapeutically effective dose of an inhibitor of TRAPS to tissue positioned adjacent to the implantable device.

A further embodiment is a medical device formed from a biomaterial comprising an anti-TRAP agent.

Yet another embodiment is a system comprising a fluid delivery tube, and a filter formed in the fluid delivery tube configured to remove a TRAP-inducing agent from a medical biofluid prior to delivery of the medical biofluid to a mammal.

Another embodiment is a method of lowering the concentrations of a TRAP-inducing agent in a medical biofluid configured for delivery to a mammal, comprising filtering the medical biofluid to remove the TRAP-promoting agent prior to delivery to the mammal.

Yet another embodiment is a system for detecting TRAPs induced by the presence of a medical device, a medical biofluid and/or a medical biomaterial, comprising: a biological tissue specimen having a pouch in the skin that is created by air, other gases, saline or a biologic buffer, gels, natural or synthetic matrix. The pouch is configured to receive the medical device, medical biofluid and/or medical biomaterial, and an imaging apparatus configured to image a portion of the biological tissue specimen at measured time intervals after insertion of the medical device to detect TRAPs and/or netosis. In embodiments, the liquids are sterile and pyrogen free.

Another embodiment is a method, comprising obtaining a biological tissue specimen, forming an air pouch in the biological tissue specimen configured to receive a medical device, a medical biofluid and/or a medical biomaterial, inserting the medical device, medical biofluid and/or a sample of the medical biomaterial in the air pouch, imaging a portion of the biological tissue specimen at multiple time intervals at a resolution sufficient to detect TRAPs, and detecting the presence of TRAPs in the biological tissue specimen adjacent to the medical device.

A further embodiment is a system for detecting the effectiveness of an anti-TRAP agent disposed proximate a medical device, a medical biofluid and/or a sample of a medical biomaterial, comprising: a biological tissue specimen having an air pouch formed therein configured to receive the medical device, the medical biofluid, and/or the sample of the medical biomaterial, and to receive the anti-TRAP agent, and an imaging apparatus configured to image a portion of the biological tissue specimen at measured time intervals after insertion of the medical device, medical biofluid and/or medical biomaterial, and the anti-TRAP agent in order to detect TRAPs.

Another embodiment is a method, comprising obtaining a biological tissue specimen, forming an air pouch in the biological tissue specimen configured to receive a medical device, a medical biofluid and/or a sample of a medical biomaterial, and to receive an anti-TRAP agent, inserting the medical device and the anti-TRAP agent in the air pouch, imaging a portion of the biological tissue specimen at multiple time intervals at a resolution sufficient to detect TRAPs, and detecting the presence of TRAPs in the biological tissue specimen adjacent to the medical device, medical biofluid and/or sample of medical biomaterial.

A further embodiment is a method of detecting TRAPs resulting from trauma induced by insertion of a medical device, a medical biofluid, and/or a medical biomaterial, comprising obtaining a biological tissue specimen, forming an air pouch in the biological tissue specimen configured to receive the medical device, the medical biofluid and/or the medical biomaterial, and inserting the medical device, medical biofluid and/or a sample of the medical biomaterial in the air pouch. The method also comprises imaging a portion of the biological tissue specimen at multiple time intervals at a resolution sufficient to detect TRAPs, and detecting the presence of TRAPs in the biological tissue specimen adjacent to the medical device.

### BRIEF DESCRIPTION OF THE DRAWINGS

FIG. TA summarizes methods and products for prolonging use of implants and reducing inflammation at implantation sites in accordance with embodiments described herein.

FIG. 1B illustrates netosis plus TRAPs in a nucleated cell.

FIG. 2 briefly describes netosis, NETs and TRAPs.

FIG. 3 briefly describes and illustrates netosis.

FIG. 4 describes the role of netosis in causing inflammation and fibrosis.

FIG. 5 shows biochemical pathways of NET and TRAP formation.

FIG. 6A describes measuring the quantity of NETs over time.

FIG. 6B also describes measuring the quantity of NETs over time.

FIGS. 7A-7E show TRAP-containing tissue visualized using various analytical techniques.

FIGS. 8A-8D illustrate TRAPs in mouse tissue.

FIG. 9 schematically illustrates the intersection of immunity and netosis.

FIG. 10 describes NET- and TRAP-induced tissue damage in diabetic patients.

FIG. 11 describes research connected to embodiments disclosed herein.

FIGS. 12A-12F show photomicrographs of TRAPs induced by insulin and/or insulin preservatives.

FIGS. 13A-13F show in vivo insulin infusion histology.

FIGS. 14A-14C show m-cresol infusion in porcine tissue.

FIGS. 15A-15C shows insulin fibril injection in porcine tissue.

FIGS. 16A-16B show TRAPs appearing in vivo in tissue adjacent an infusion cannula.

FIGS. 17A-17E show TRAPs appearing in vivo in tissue adjacent an infusion cannula on day 7 traps are indicated by gray and white arrows indicated in H&E stain tissue section.

FIGS. 18A-18B show neutrophil TRAP formation on infusion cannulas in vitro.

FIG. 19 illustrates detection of neutrophil-derived TRAPs on sensors in vitro.

FIGS. 20A-20D show TRAPs in tissue at an in vivo glucose sensor implantation site.

FIG. 21 shows TRAPs in tissue at an in vivo glucose sensor implantation site.

FIG. 22 shows tissues at an in vivo glucose sensor implantation site using a commercially available sensor.

FIG. 23 shows tissues at an in vivo glucose sensor implantation site using a commercially available sensor.

FIG. 24 shows tissues at an in vivo glucose sensor implantation site using a commercially available sensor.

FIG. 25 shows tissues at an in vivo glucose sensor implantation site using a commercially available sensor.

FIG. 26 shows neutrophil TRAPs on a suture in vitro.

FIG. 27 shows commercially available surgical mesh products.

FIG. 28 shows commercially available surgical mesh products.

FIG. 29 shows NETs on surgical mesh fibers.

FIG. 30 shows NETs on surgical mesh fibers.

FIG. 31 shows NETs on surgical mesh fibers.

FIG. 32 shows NETs on surgical mesh fibers.

FIG. 33 shows TRAPs at biomaterial implantation sites in vivo.

FIG. 34 shows TRAPs at biomaterial implantation sites in vivo.

FIG. 35 summarizes several solutions to overproduction of TRAPs and NETs, which are described in detail below.

FIG. 36 shows data regarding the use of an air pouch to detect TRAPs on the surface of an implanted device.

FIG. 37 illustrates TRAPs in an air pouch.

FIG. 38 shows TRAP formation after a 4-day continuous infusion of phenol and m-cresol.

FIG. 39 shows insulin fibril in phenolic preservative injection once a day over 7 days.

FIG. 40 shows antibodies used and treatments for human PMN TRAPs staining.

FIG. 41 shows human PMN TRAPs staining.

FIG. 42 shows human PMN TRAPs staining.

FIG. 43 shows human PMN TRAPs staining.

FIG. 44 describes macrophage staining of swine macrophage cells.

FIG. 45 illustrates the stained samples.

FIGS. 46-58 describe and illustrate neutrophil TRAP study triplicates.

FIGS. 59-60 show the appearance of TRAPs over time in insulin and insulin preservatives as compared to controls.

FIG. 61 shows a commercially available matrix from Sigma.

FIG. 62 depicts insulin degradation by PMN TRAPs.

FIG. 63 lists some triggers for TRAP formation.

FIG. 64A provides a list of articles describing the air pouch method.

FIG. 64B illustrates one embodiment of the air pouch model.

FIGS. 65A-67 shows and describes the results of experiments using air pouches.

FIGS. 68-71 are tables showing TRAP inhibitors.

FIGS. 72A and 72B show an exemplary insulin pump with an in-line filter.

FIGS. 72C and 72D shows an exemplary syringe for delivery of a liquid, such as insulin, with a filter to remove components such as preservatives.

FIG. 73A shows devices and methods for removing preservative and antimicrobial agents from insulin, or for removing components from other medical bio-fluids at the time of infusion.

FIG. 73B shows devices and methods for removing fibrils and other TRAP-inducing components during CSJI or infusion of other medical bio-fluids.

FIG. 73C shows devices and methods for delivering drugs, factors and/or agents that prevent or reverse TRAP formation in vitro or in vivo.

FIG. 73D shows methods and a dual lumen cannula for delivery of a drug, factor and/or agent during CSJI or infusion of other medical bio-fluids that can prevent TRAP formation, reverse TRAP formed in situ, and/or treat tissue reactions induced by TRAPS.

FIG. 73E shows methods and devices, such as cannulas or other medical devices or biomaterials, to increase biocompatibility and/or prevent infections and/or netosis, which would otherwise result in TRAP-induced tissue reactions in vivo.

FIG. 73F shows additional methods and devices to increase biocompatibility of cannulas and/or prevent infections and/or netosis resulting in TRAPS-induced destructive tissue reactions in vivo.

FIG. 73G shows methods and devices to make cannulas and collars more biocompatible and/or prevent infections and/or netosis and TRAP formation.

FIG. 73H shows further methods and devices to make cannulas more biocompatible and/or prevent tissue death, and to prevent TRAP formations from infusion of medical bio-fluids and or medical bio-materials.

FIG. 73I shows additional methods and devices to make cannulas more biocompatible and/or prevent infections, and to prevent TRAP formation or to remove TRAPs.

FIG. 73J shows additional methods and devices to make cannulas more biocompatible and/or prevent infections, and to prevent TRAP formation or to remove TRAPs.

FIG. 74A shows a conventional cannula.

FIG. 74B shows cannulas incorporating filters and/or absorbing materials to prevent TRAP-inducing tissue reactions and/or promote TRAP degradation.

FIG. 74C shows cannulas incorporating filters and/or absorbing materials to prevent TRAP-inducing tissue reactions and/or promote TRAP degradation.

FIG. 74D shows cannula incorporating filters and/or absorbing materials to remove TRAP-inducing agents.

FIG. 74E shows a cannula incorporating filters and/or absorbing materials to remove TRAP-inducing agents.

FIG. 74F shows a cannula incorporating filters and/or absorbing materials to remove TRAP-inducing agents.

FIG. 75A shows a conventional cannula.

FIGS. 75B-75F schematically show systems which incorporate drugs, factors and/or other agents, such as anti-TRAP agents, to improve biocompatibility of cannulas.

FIG. 76A shows a conventional syringe used to deliver insulin.

FIGS. 76B-76E show syringes incorporating filters and/or absorbing materials.

FIG. 77 a-f are photomicrographs of human neutrophils (cells from human blood) in culture dish with negative control media (a-c), or positive control (A23187) (d-f) that triggers TRAPs induced by neutrophils. TRAPs appear as white dots and clumps in FIG. 78 (e) (fluorescence) and merged images (780 in the A23187 neutrophils).

FIG. 78 a-c. Photomicrographs of human neutrophils (cells from human blood) in culture dish including a sensor without the presence of white dots and clumps indicating that sensor do not release any TRAP-inducing substance when cultured with neutrophils.

FIG. 79 a-f. Demonstrates that commercial sensor 1 without neutrophils in media plus addition of sytox green show lack of TRAPs (79a-c). Addition of neutrophils in media plus sytox green to sensor show appearance of white clumps of TRAPs (79 e and f).

FIG. 80 a-f Demonstrates that commercial sensor 2 without neutrophils in media plus sytox green show lack of TRAPs (80a-c). Addition of neutrophils to media plus sytox green and sensor, show appearance of white clumps of TRAPs (80 e and f).

FIG. 81 a-f. Demonstrates that commercial sensor 3 without neutrophils in media plus sytox green, show lack of traps (81a-c). Addition of neutrophils in media plus sytox green to the sensor show white clumps of traps appearance (81 e and 0.

FIG. 82 a-f. demonstrates that commercial sensor 4 without addition of neutrophils in media plus sytox green lack TRAPs presence (FIG. 82a-c). However, when neutrophils in media plus sytox green are added to the sensor, white clumps of traps appear (FIG. 82 e and f).

FIG. 83 a-o shows TRAPS formed on a commercial glucose sensor after implantation in a non-diabetic human subject for 24 hours. Upon removal from the tissue, sensors were placed in tissue dish with media plus sytox green and photographed for presence of TRAPs. Traps are clearly present across the entire sensor surface (83 f-j fluorescent image) and 83k-o (merged image) FIG. 84 a-o shows TRAPS formed on a commercial glucose sensor after implantation in non-diabetic human subject A for 24 hours. Upon removal from the tissue, sensors were placed in tissue dish with media plus sytox green and photographed for presence of traps. TRAPs are clearly present across the entire sensor surface (84 f-j fluorescent image) and 84 k-o (merged image) FIG. 85 a-g shows TRAPS formed on a commercial glucose sensor after implantation in a non-diabetic human subject B for 24 hours. Sensors were removed and placed in tissue dish with media plus sytox green and photographed for presence of TRAPs. TRAPs are clearly present across the entire sensor surface (85 f-j fluorescent image) and 85 k-o (merged image) FIG. 86a-o shows TRAPS formed on a commercial glucose sensor after implantation in a human subject C for 24 hours. Sensors were removed and placed in tissue dish with media plus sytox green and photographed for presence of TRAPs. TRAPs are clearly present across the entire sensor surface (86 b, e, h, k, n: fluorescent image) and 86-c,f, i, l o (merged image)

FIG. 87 depicts a prototype insulin pen and needle in accordance with embodiments described herein which can remove phenolic preservatives from commercial drugs such as commercial insulin formulations.

FIG. 88 shows data from an in vivo experiment in which an Abbott sensor was placed in porcine tissue for 24 hours, the implantation site was punch biopsied, fixed and the resulting tissue was processed, sectioned and stained by H&E dye. Presence of TRAPs are localized in dark bands (rivers of traps in the tissue next to the glucose sensor tip.

FIG. 89 further illustrates the methods of preparation and evaluation of murine air pouch model shown in FIGS. 64A-64B.

FIG. 90 further illustrates the air pouch model shown in FIGS. 64A, 64B and 89. Which includes injection and evaluation of liquids, biomaterial and medical devices using air pouch model.

FIG. 91 schematically shows a prototype of another type of modified drug-injecting pen, such as insulin pen, in accordance with embodiments disclosed herein.

FIG. 92 shows the presence of TRAPS in porcine skin over a period of 21 days when a sensor has been implanted.

FIG. 93 shows H&E, Trichrome and CD31 (vessel staining) for the porcine skin, for the samples tested to obtain the data shown in FIG. 92.

FIG. 94 shows a workflow for in vitro cell testing using an air pouch model.

FIG. 95 provides lists of non-limiting examples of biomarkers and inhibitors that can be used in the workflow shown in FIG. 94.

FIG. 96 shows a workflow for in vivo mammal testing using an air pouch model.

FIG. 97 provides a list of mammal types that can be used in the testing procedure workflow shown in FIG. 96.

### DETAILED DESCRIPTION

The inventors believe that at the core of the failure of many medical devices, medical bio-fluids and/or medical bio-materials in vitro, in vivo, ex vivo or extracorporeally, is the tissue destructive reactions (inflammation, loss of blood vessels and fibrosis) triggered by the insertion of the medical devices, medical bio-fluids and/or medical bio-materials into tissue site(s). The tissue destructive reactions result from the devices, bio-fluids and/or biomaterial triggering a specific and unique process of cell death, known as netosis. Netosis is a form of cell death in which there is formation of netosis induced “TRAPs”. These TRAPs are a specialized form of extracellular DNA, that is triggered in injured/dying/dead nucleated cells containing DNA, in response to various types of cell injury. This injury can be induced by microbes, microbial products or some non-microbial agents. Recently, the inventors have demonstrated within this application, that medical devices, medical bio-fluids and medical bio-materials can trigger netosis and TRAP formations in vitro and in vivo. They further demonstrate within this application that medical devices, medical bio-fluids and medical bio-materials including insertion into tissue induced netosis and TRAPs that trigger tissue reactions (inflammation, loss of vasculatures and fibrosis). The inventors further believe that medical devices, medical bio-fluids and medical bio-materials induced netosis and TRAP formation with associated inflammation, loss of vasculatures and fibrosis, limit effective treatment and lifespan of these medical devices, medical bio-fluids and medical bio-materials. This includes in vitro, in vivo, ex vivo and extracorporeal interactions of medical devices, medical bio-fluids and medical bio-materials with cells, tissues, biologic fluids (e.g., blood and fluids).

The Inventors further believe that the netosis/TRAP induced tissue reactions resulting is specific biomarkers that can be detected in cells, tissue, and biologic fluids e.g., blood and urine, that correlate with the presence of netosis, as well as TRAP and TRAP products useful in diagnosis, treatment and prognosis of tissue reactions and outcome induced by medical devices and medical bio-fluids and medical bio-materials, but can also be used to screen medical device, biofluids and medical bio-materials used in medical devices and treatment in vitro and in vivo, to determine their safety and utility for use in the treatment to disease and medical problems for humans and animals.

The inventors further believe that agents and materials that prevent (inhibit) or minimize netosis and TRAP formation, as well as substances can remove TRAPs after they have formed, improve functionality of medical devices, medical bio-fluids, and medical bio-materials.

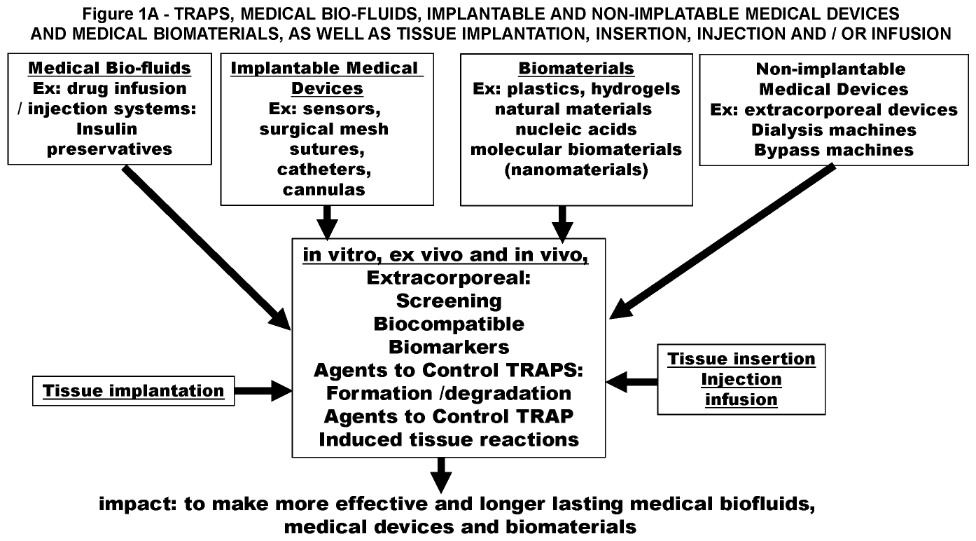

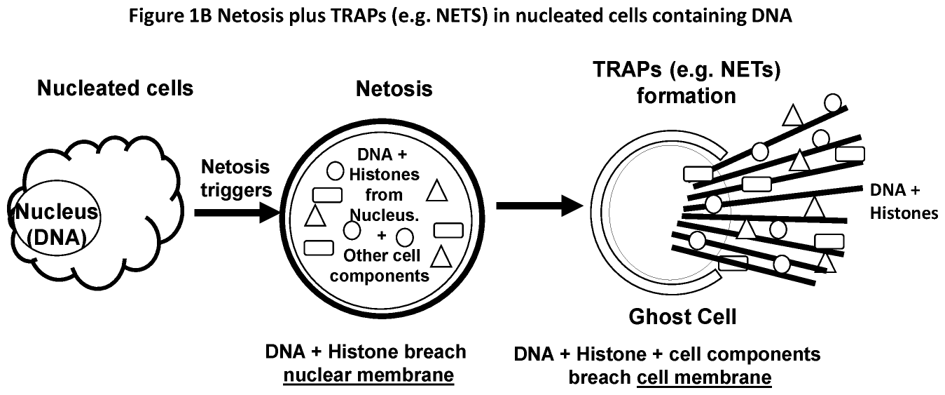

Substances that inhibit netosis/TRAP associated factors (e.g. oxygen radicals, PAD4, cell receptors, proteases (e.g. elastase)), as well in cell and tissue reactions triggered by netosis, TRAPs and related cell and tissue reaction increase the accuracy, function, effectiveness and/or functional life of medical devices, medical bio-fluids and medical bio-materials in vitro, in vivo, ex vivo and extracorporeally. Substances such as DNase can be used to remove TRAPs after they have formed. The inventors believe that increasing the functional lifespan and accuracy/effectiveness of medical devices, medical bio-fluids and medical bio-materials benefit patients and animals needing treatment for a disease. FIG. 1B provides a summary overview of netosis and TRAP formation.

Netosis and TRAPs, were initially discovered in association with microbial diseases (infections) and the interactions with microbes (e.g., bacteria and viruses) and their products (e.g., microbial toxins) with cells, biologic fluids and or tissues in vitro and in vivo. Netosis and TRAPs resulting from the interaction of microbes with biologic fluids, cell and tissues, have been implicated in the body's effort to clear microbes and infections (e.g., bacteria and viruses) or microbial products (e.g., endotoxins) from infected tissues and biologic fluids (blood, urine, etc.).

Additionally, netosis, TRAPs and TRAP derived products, have been used as biomarkers of microbial disease (i.e., infections) and can be used as biomarkers of infection related disease progression, treatments and prognosis. Currently, medical devices, medical bio-fluids and medical bio-material triggered netosis and TRAPs biomarkers, have not be identified or used as biomarkers for medical devices, medical bio-fluids and medical bio-material induced netosis and or TRAP formation, as well as correlating with progression, treatments and prognosis tissue reactions and the function and lifespan of medical devices, medical bio-fluids and medical bio-material.

Additionally, the inventors believe that panels of common and unique biomarkers induced by microbes, microbial products, medical devices, medical bio-fluids and medical bio-material, as well as biomarkers of inflammation, loss of vasculatures and fibrosis can be used in combination to better understand, detect, prognosis and treat, netosis and TRAP induced tissue reactions in vitro, in vivo, ex vivo and extracorporeally and to better treat patients with diseases that require the uses of medical devices, medical bio-fluids and medical bio-materials to assure the most efficient, effective and extended lifespan of medical devices, medical bio-fluids and medical bio-material.

In addition to microorganisms, netosis is triggered by other various stimuli, including proinflammatory cytokines (TNF-ca, IL-8), platelets, activated endothelial cells (eCs), nitric oxide, monosodium urate crystals, and various autoantibodies.

To function effectively, medical devices (e.g., implantable, extracorporeal and in vivo medical devices) and medical bio-fluids (e.g., insulin) and medical bio-materials used in the treatment of disease, need to be biocompatible in vitro, in vivo, and ex vivo or extracorporeal. Therefore, to be biocompatible, it is important that medical devices, medical bio-fluids and medical bio-materials themselves are not inherently toxic and/or kill/damage cells or tissues in vitro, in vivo, ex vivo, extracorporeally. These devices, biomaterials or biofluids must not trigger tissue reactions directly or indirectly, such as through the activation of biologic fluids as is the case for complement or clotting system activation, resulting in destructive tissue reactions in vivo (e.g., inflammatory, edema, and wound healing process (scarring with loss of vasculatures)) that cause damage or tissue reaction when coming in contact with cells, biologic fluids (blood or urine, or tissues or organs). Non-biocompatible medical devices, medical bio-fluids and medical bio-materials induced reactions can not only destroy cells, tissues and organs, this lack of biocompatibility can also limit the performance or functionality pertaining to the lifespan of the medical devices, medical bio-fluids and medical bio-materials, which undermines the treatment of the underlying disease. Thus, screening of medical devices, medical bio-fluids and medical bio-materials for their biocompatibility, is important in deciding which of these medical devices, medical bio-fluids and medical bio-materials can be used in the design, fabrication and/or testing of medical devices, medical bio-fluids and medical bio-materials for use in treating of diseases that can benefit from the uses of medical devices, medical bio-fluids and medical bio-materials in treating disease in humans or animals. For example, with diabetes, it is important for the devices (e.g., glucose sensors, insulin infusion devices), as well as medical bio-fluids (e.g., insulin) to be biocompatible in vivo to allow determining accurate blood glucose levels, and the injection or infusion of the proper amounts of insulin, needed to control blood glucose levels.

Little is known about the roles of netosis, TRAPs and TRAP formation/composition, as they relate to medical devices, medical bio-fluids (e.g., drug delivery systems and agents including preservatives), and biomaterials (liquids and solids) used to construct these devices and medical bio-fluids, as well as the impact of netosis/TRAP induced tissue reactions on their function in vitro, in vivo or extracorporeally in the short and long run in vivo. Additionally, little is known about the composition and function of netosis, and TRAPs induced by medical devices, medical bio-fluids and medical biomaterials, when compared to netosis and TRAPs triggered by microbes (bacteria and viruses). Additionally, little is known about the agents that can detect, suppress netosis, TRAP formation and associated tissue reactions, including agents that prevent, suppress or even enhance netosis and TRAPs in response to medical devices, medical bio-fluids and medical bio-materials in vitro, in vivo, ex vivo as well as extracorporeally, as well as preventing loss of biocompatibility or bioactivity or loss of performance of medical devices, medical bio-fluids or bio-materials in vitro, extracorporeally, ex vivo and in vivo.

Specific, non-limiting examples of implantable devices impacted by netosis and TRAP driven tissue reactions.
1. Implantable medical devices, including but not limited to sensors, medical bio-fluids infusion systems and delivery sets, including drug infusion systems, cannulas, catheters, surgical mesh, sutures, stents, implantable valves, clips, pacemakers, bags, grafts, filters, patches, locks, wires, ligatures, screws, shunts, connectors, adapters, stimulators, fasteners, plates, rods, pins, fasteners, nuts, bolts, washers, staples, nails, caps, rings, expanders, electrodes, ports, bone graft materials, spermatocele, tape, wax, wraps, balloons, barriers, cement, scaffolds, vessel guards, plugs, surgical films, and other FDA authorized implantable medical devices.
2. Non-implantable medical devices, including needles, such as needles or syringes, tubing for dialysis, tubing and membranes for bypass machines, including machines for cardiac bypass surgery, tubing for blood transfusions, chambers, non-implantable valves, clips, filters, molds, dialyzers, pumps, sensors, tape, wax, wraps, and other FDA authorized non-implantable medical devices. Products such as bandages also can be deemed non-implantable medical devices.

### Overview

As is summarized in FIG. 1A, the present application describes netosis and/or TRAP-based platforms, assays, markers, reagents and agents that can:
1) Be used to screen medical devices (implantable or non-implantable medical devices), bio-fluids (e.g., drug delivery systems, agents and fluids including their preservatives), and/or biomaterials to predict/demonstrate biocompatibility including in vitro, ex vivo, in vivo and extracorporeal biocompatibility.
2) Evaluate netosis and/or TRAPs and their related products including, injured, dying and dead cells and tissues, including detecting netosis and or TRAPs in vitro, ex vivo and in vivo and extracorporeal, and/or inherently lack of biocompatibility resulting in tissue destruction, inflammation, fibrosis, loss of vessels, e.g., air-pouch model for detect of netosis and or TRAPs.
3). Identify and quantify biomarkers and functional markers of netosis and or TRAPs induced by bio-fluids (e.g., drug delivery systems, agents and including its preservatives), medical devices, and biomaterials, which are specific and unique to the inducing material, device, drug delivery fluid or drug delivery agents.
4). Identify local and circulating products/biomarkers from netosis and or TRAPs/nets, as well as netosis and or TRAP-derived products that can cause disease, and/or predict the status of tissues reactions induced by bio-fluids, medical devices, and biomaterials used in implantable and non-implantable medical devices for their respective in vitro, ex vivo and in vivo.
5). Identify new (discovery and validation of chemicals, molecules, liquids & materials) and test existing agents that can:
   a) Inhibit or alter netosis and or TRAP formation, stop netosis and/or speed TRAP degradation in vitro, in vivo, ex vivo, or extracorporeally,
   b) Inhibit or alter netosis and TRAP related products (e.g., products of injured, dead and dying cells) in vitro, ex vivo and in vivo,
   c) Degrade formed or forming TRAPs, to minimize their destructive tissue reactions (e.g., inflammatory and wound healing process (scarring) to these TRAPs and related cell/tissue products, for example uses of DNase 1 to degrade DNA based TRAPS,
   d) Prevent circulating netosis and TRAP products from causing local or systemic disease, Neutralize netosis/TRAP/DNA associated factors e.g. proteases (e.g. alpha-1-trypsin inhibitor which can inhibit elastase), inhibitors of oxygen radial related factors (inhibitors of MPO, catalases, as well as radical oxygen species (ROS) scavengers),
   e) Be employed in conjunction with medical bio-fluids, medical devices, and medical bio-materials used in implantable and non-implantable medical devices for their respective in vitro, ex vivo in vivo and extracorporeal uses.
   f) Prevent netosis and or TRAPs formed, trigger the release of toxic or proinflammatory factors for living, dying or dead cells, which would induce additionally cell death, netosis, tissue, protein, nucleic acids (e.g. DNAs or RNAs) lipid degradation/alteration or chemical reactions that would promote cell death, cell injury, TRAP generation, inflammation, loss of blood vessels or fibrosis, or damage to structure components of cells and or tissues. This using inhibitors of pro inflammatory agents such as cytokine inhibitor, promotors od blood and lymphatic vessel formation and function (e.g. VEGFa, VEGFb, VEGFc, and VEGFd, which induce new blood and lymphatic vessel formation), inhibitors of fibrosis (e.g. such as anti TGFB cytokines such as antibodies and TGFB receptor inhibitors).

### Definitions

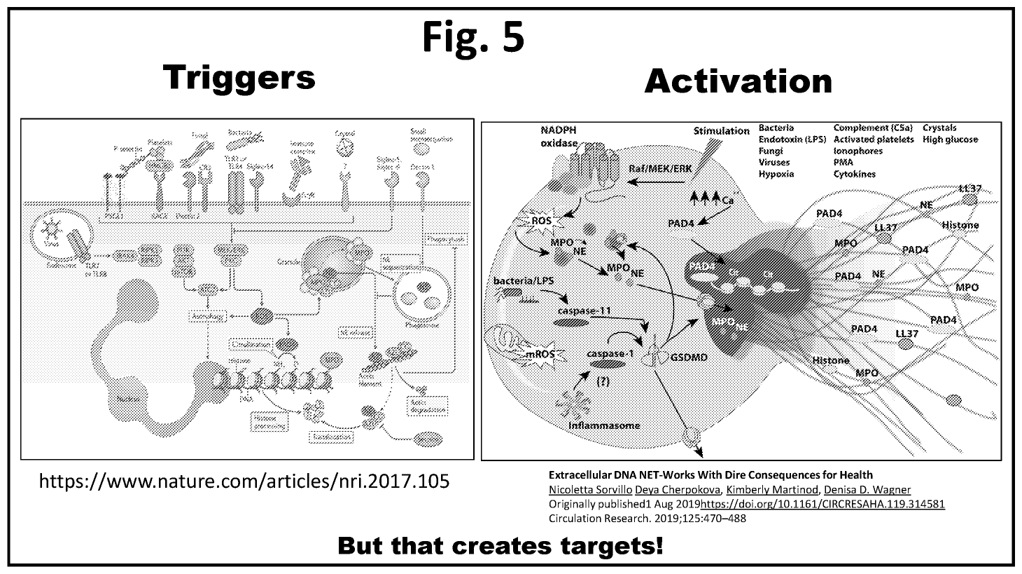

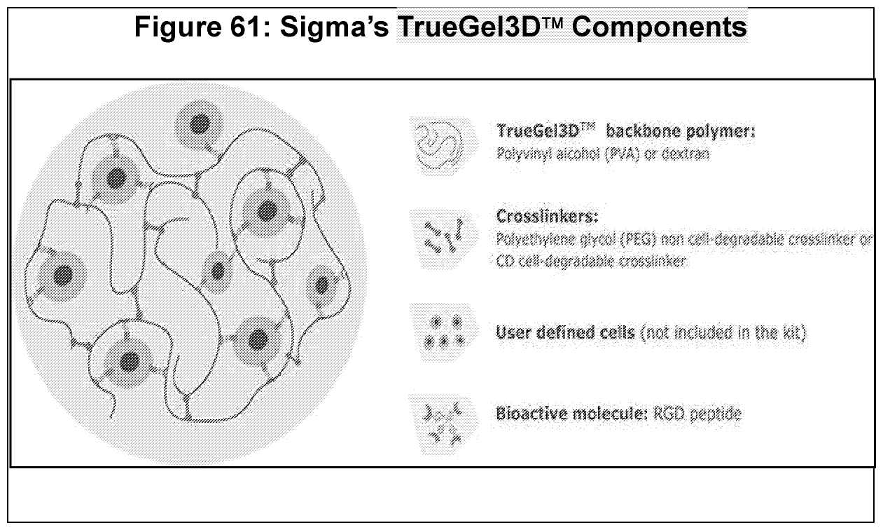

A medical device: A medical device is an instrument, apparatus, implant, machine, tool, material, substance, chemical, biological substance, in vitro reagent, or similar article that is to diagnose, prevent, mitigate, treat, or cure disease or other conditions, and, unlike a pharmaceutical or biologic, achieves its purpose by physical, structural, or mechanical action, but not through chemical or metabolic action within or on the body (this separates devices from drugs). Medical devices are devices used in vitro, in vivo, ex vivo, in situ or extracorporeally, to detect, treat or monitor diseases in organisms.
An implantable medical device: implantable medical device, is a medical device is an instrument, apparatus, implant, machine, tool, in vitro reagent, or similar article that is to diagnose, prevent, mitigate, treat, or cure disease or other conditions, and, unlike a pharmaceutical or biologic, achieves its purpose by physical, structural, or mechanical action, but not through chemical or metabolic action within or on the body (this separates devices from drugs). Examples sensor, cannulas catheters, stents, mesh. Implantable medical devices can come into contact with living cells, tissues and fluids) or structures like bone or matrix such as collagen as part of treatment.
Insertion and insertion devices: includes any type of insertion mechanism, type or delivery mechanism to insert or delivery the medical device into the tissue.
A non-implantable medical device: non-implantable medical device is a medical device (an instrument, apparatus, implant, machine, tool, in vitro reagent, or similar article) that is to diagnose, prevent, mitigate, treat, or cure disease or other conditions, but comes in contact with blood, urine tissue and/or cells, and by doing so, induce formation of TRAPs (e.g. NETs) in vitro and/or in vivo. The resulting NETS and/or TRAPs can in turn cause tissue reactions and/or acute and chronic disease in humans and animals but, unlike a pharmaceutical or biologic, achieves its purpose by physical, structural, or mechanical action, but not through chemical or metabolic action within or on the body (this separates devices from drugs).
A medical bio-fluids: includes cells, viruses, natural/biological substances, synthetic substances, drugs, recombinant proteins, lipids, nucleic acids, therapeutic RNAs and DNAs, nanoparticles, liposomes, as well as biologicals including blood, plasma, serums, cell components including but not limited to micro vesicles, exosomes liquids, gels, oils, emulsions, that can or are thought to help in treating disease and/or symptoms associated with diseases. They can be used by injection or infusion or contact with cells or tissue. Medical biofluids include drugs such as insulin. Commercial insulin formulations contain preservatives such as phenol and m-cresol.
A medical bio-material: natural or synthetic solids, nets, particles, nanoparticles, liposomes, gels, high viscosity oils, that may be used directly or indirectly (e.g. support treatment modalities) for the treatment of disease, including minimizing of symptoms used in treating mammals or other living organisms, to improve their quality and/or length of life or used to construct medical devices and/or medical bio-fluids that that can directly or indirectly treat diseases. Medical bio-materials are natural or synthetic or combinations of natural plus synthetic materials used to manufacture medical devices, examples including but not limited to sensors, drug infusion systems/delivery sets (e.g., insulin infusion set), tubing and membranes used in bypass machines, surgical mesh, sutures, catheters, cannulas, needles and stents. Medical biomaterials include silicones, synthetic hydrogels, biological hydrogels, etc.
Also, a medical bio-material is used to construct implantable and non-implantable medical device, hold, transport, infuse, injector remove medial bio-fluids form cells, tissues organs or other areas or surfaces of a living organism.
Also, any combination of the above medical devices, medical bio-fluids and medical bio-materials and any of the substances, agent, cell, replicating substances (e.g., viruses) nucleic acid etc. described above.
Air pouch model: an experiment animal model; in which air/gases, saline or biologic buffer, gels natural or synthetic matrix is injected under the skin, to create a compartment (pouch) in which substances are injected to determine toxicity of the test substance by removing the substances and evaluating them in vitro, lavaging (washing out) the contents of the pouch with fluids or gels evaluating them in vitro, and then characterizing and quantifying the contents of the lavage e.g., inflammatory cell/factors levels and types see FIG. 61A (see, e.g., the three articles cited below). Tissue from the pouch can be removed and components of the pouch can be evaluated in vitro including but not limited to staining of tissue cells and other components (e.g. metrics or presents of biologic on non-biologic factors. Blood, tissue and fluids can be non-pouch areas of the animal and also evaluated. Inducers and inhibitors of tissue reaction can be injected, implanted, inhaled or feed to the animal at any timepoint during the testing to determine the impact of these substance have on any tissue, matrix, cell or factor in the mouse.
The contents of the following articles authored by the inventors is incorporated by reference herein in its entirety.
1. Advancing continuous subcutaneous insulin infusion in vivo: new insights into tissue challenges. Kesserwan S, Mulka A, Sharafieh R, Qiao Y, Wu R, Kreutzer D L, Klueh U. J Biomed Mater Res A. 2021 July; 109(7):1065-1079. doi: 10.1002/jbm.a.37097. Epub 2020 Sep. 18. PMID: 32896081
2. Insulin Derived Fibrils Induce Cytotoxicity in vitro and Trigger Inflammation in Murine Models. Lewis B E, Mulka A, Mao L, Sharafieh R, Qiao Y, Kesserwan S, Wu R, Kreutzer D, Klueh U., J Diabetes Sci Technol. 2021 Jul. 21: 19322968211033868. doi: 10.1177/19322968211033868. Online ahead of print. PMID: 34286629
3. A pharmacological approach assessing the role of mast cells in insulin infusion site inflammation. Kesserwan S, Mao L, Sharafieh R, Kreutzer D L, Klueh U., Drug Deliv Transl Res. 2021 Sep. 24. doi: 10.1007/s13346-021-01070-w. Online ahead of print. PMID: 34561836
   TRAP—As used herein, “TRAPs” refers to networks of extracellular DNA based “fibers” or DNA meshes/nets that are released from cells or subcomponents of cells e.g. mitochondria. In some cases, TRAPs are triggered by microbes or their products, which bind microbes and trigger tissue reaction to eliminate the microbes and their products. It other cases TRAPs can be triggered by diseases such as autoimmune diseases. As presented in this application these inventors demonstrate the TRAPs can be induced in cells that contain DNA in their nucleus, can be triggered by medical devices, medical bio-fluids or bio-materials as described with in this application as well as neutralized prevented and or removed as described in this application. As used herein, the term anti-TRAP agent refers to aa a TRAP inhibitor, which is an agent that can prevent, suppress, and or degrade one or more components of a TRAP, e.g., DNA, ROS, proteases, MPO to name but a few), or disrupts the pathway of TRAP formation, thereby preventing the formation of TRAPs. Additionally, these inhibitors can prevent netosis and TRAPs by blocking biological and chemical pathways that induce netosis and or TRAPs, or induce or suppress biological or chemical pathways that can trigger biological, chemical or cellular pathways the induce cell and tissue damage, as well as promote inflammation, loss of vascular networks and or induce fibrosis in a living organism. A TRAP inhibitor can inhibit expelled components and/or biochemical pathways inside cells)
   NET—“NET” refers to a type of TRAP that is induced/released from neutrophils (i.e. neutrophil extracellular TRAP (NET). They are given the name of NETs because they are extracellular DNA from neutrophils. The process of forming toxic pathologic extracellular DNA from neutrophils (NETs) is referred to as netosis. So, TRAPs from macrophages are sometime referred to as mac-TRAPs or Mets, mast cell are referred to MC TRAPs or MCets, etc. TRAPs can trigger in all mammalian cells in response to cell activation or cell death. All traps have similar composition including DNA backbones with cell and tissue components bound/decorated on the DNA backbone. Therefore all NETs are TRAPs, but not all TRAPs are NETs.
   As used herein, the term “anti-TRAP agent” refers to substances that inhibit the formation of TRAPs, substances that degrades one or more components of a TRAP after it has formed, and substances that neutralize toxic factors that are present on TRAPs.
   As used herein, the term “anti-NET agent” refers to a NET inhibitor, which is an agent that degrades one or more components of a NET, or disrupts that pathway of formation, thereby preventing the formation of NETs. NETs are just a subset of TRAPs based on the cell that undergoes netosis and releases the TRAP, all nucleated cells, in which the nuclear material contains DNA can make TRAPS.
   Netosis inhibitor and anti-netosis agent—As used herein, the terms “netosis inhibitor” and “anti-netosis agent” refer to any natural or synthetic substance that is able to (1) prevent binding of triggers to cell receptors, (2) prevent cell activation pathways that induce/produce NETs (see, for example, FIG. 5), and/or (3) inhibit cell entry of netosis inducing agents that trigger netosis directly (for example, diffusing directly into the cells), and/or (4) inhibit cell entry of netosis inducing agents indirectly (for example, binding through a surface receptor or molecule). A netosis inhibitor can inhibit expelled components and/or biochemical pathways inside cells).
   Biomarker—As used herein, the term “biomarker” means any agent that detects biological processes and/or can be used for diagnosis, prognosis and/or treatment of diseases.

### The Problem

TRAPs (e.g., NETs, which arise from neutrophils), represent toxic or pathologic extracellular DNA, which are the product of netosis, can form in nucleated cells, for example cell injury because of the presence of microbes, including viruses and bacteria, in order to kill and or contain the microorganism. TRAPs also can form in response to microbial products (e.g., endotoxins) and non-microbial substances (e.g., PMA, ionophores). In some cases, excessive TRAP formation occurs due to a medical condition such as cancer, or an autoimmune disease such as diabetes, Antineutrophil cytoplasmic antibody-associated vasculitis (AAV), Systemic lupus erythematosus, Rheumatoid Arthritis, psoriasis, Antiphospholipid syndrome, multiple sclerosis, dermatomyositis, polymyositis (PM), autoimmune pancreatitis, or a Drug-Induced Autoimmune Disease.

The existence and roles of netosis and TRAPs for medical devices, medical bio-fluids and medical bio-material is poorly understood.

The inventors demonstrate in this application that netosis with TRAP formation can be triggered by medical devices, medical bio-fluids and medical bio-materials in vitro and in vivo. Also the inventors demonstrate that netosis and trap formation triggered by medical devices, medical bio-fluids and medical bio-materials trigger tissue reactions that not only damage tissue function and architecture, but also limit the function lifespan of medical devices, medical bio-fluids and medical bio-materials and their ability to treat disease successfully.

The medical devices and medical bio-fluids and medical bio-materials can be used directly in the treatment of disease or as delivery or support systems for treatment of diseases, such as a synthetic or biologic material, plastic, hydrogel, nucleic acid, or a molecular biomaterial such as a nanomaterial, or therapeutic nucleic therapeutic agents e.g., modified RNAs and DNAs, noncoding RNA (miRNA, siRNA etc.) Non-limiting examples of implantable medical devices include sensors, surgical mesh, sutures, catheters, cannulas, and collars for cannulas. Non-limiting examples of non-implantable medical devices include medical tubing and membranes, as is used in dialysis and blood transfusions, and bypass machines used in cardiac surgery, and kidney dialysis machines. Non-limiting examples of medical bio-fluids include drugs administered oral, by injection or infusions, insulin solutions, and other medical bio-fluids that contain active agents, or preservatives and stabilizers, and other additives in addition to active agents.

Trauma triggered in tissue by insertion, injection and/or infusion of medial biomaterials, medical bio-fluids (for example, commercial insulin solutions) and or medical devices, can result directly in cell death, damaged tissue and tissue cells, or damaged organs (insertion trauma) or as the result of the presence in or near cells, tissues, organs, fluid present in tissue or organs (blood, plasma, serum etc., all the types of trauma induce recruitment of leukocytes and fluids (blood and plasma, and serum (edema) as well as other cell types, endothelial, epithelial cells and fibroblasts to the injured site in the body. All these cells, as well as other cell populations can be injured/trigger to undergo netosis and productive in formation of extracellular DNA (TRAPs), from tissue or recruited nucleated cells, which are referred to as TRAPs.

These TRAPs, which are composed of DNA, as well as other cell and tissue components, such as histones, myeloperoxidases, proteases (e.g. elastase), from the injured and dying cells, as well as other tissue components. Both the DNA and these other DNA TRAP associated factors are cell and tissue toxic and can promote inflammation, loss of vasculatures, as well as fibrosis, all of which result in the loss of tissue functions directly and or to nearby cells and tissue. For example, TRAPs promote edema, inflammation and, fibrosis (with loss of vessels), as well as clot formation, both locally and at distant sites (e.g., local and distant tissue and blood and lymphatic vessels/vasculature, which leads to vasculature damage and tissue ischemia, tissue damages and destruction of local and distant tissue architecture and function in general, as well as loss of function of local and distant medical devices, medical bio-materials, medical bio-fluids (example commercial insulin solutions).

Relevance to Diabetes-Insulin injection/infusion remains one of the least studied, but most critical elements of an integrated artificial pancreas (AP) system (i.e., glucose sensor plus insulin infusion). Successful AP system requirements include the need to maintain precise and accurate blood glucose measurements (sensors) that control insulin infusion pumps to deliver of very minute and continuously variable amounts of insulin in response to normalize blood glucose (BG). Additionally, the physical absorption and blood glucose response to infused insulin should remain constant permitting stable AP algorithm performance. Interestingly, little was known in the past about the impact of insulin excipients/diluents and continuous subcutaneous insulin infusion (CSII) failures including loss of blood glucose regulation. Specifically, diluent provided by Eli Lilly and Company, represents phenolic preservative, which has a combined m-cresol and phenol concentration of 2.25 mg/ml.

### Solutions—General

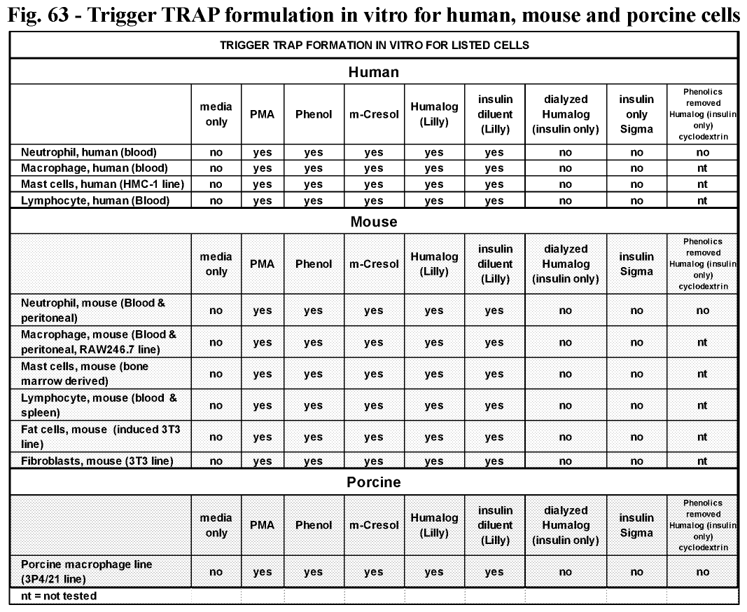

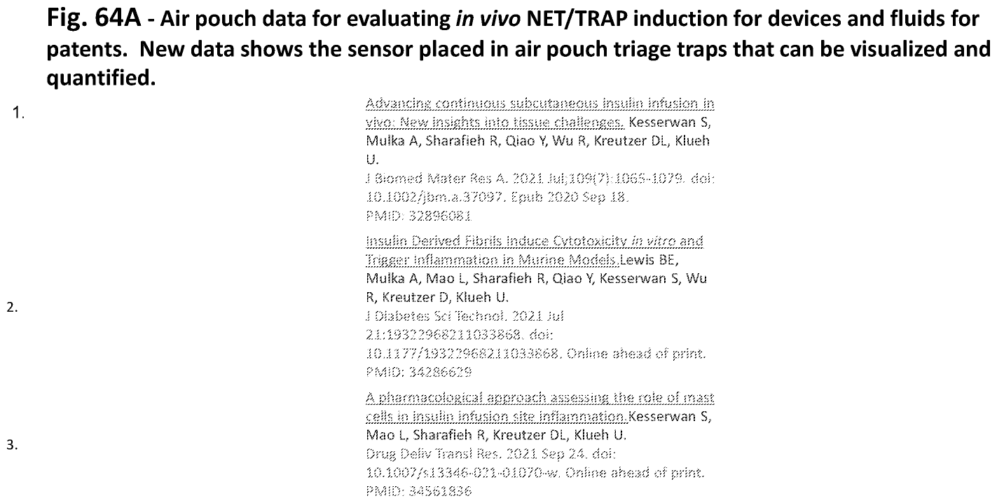

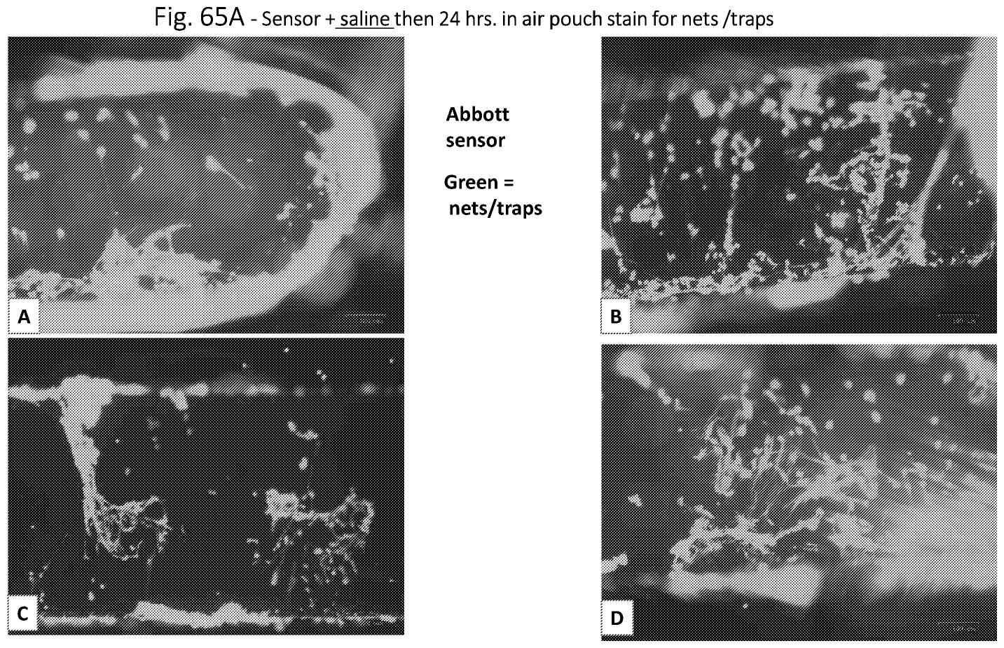

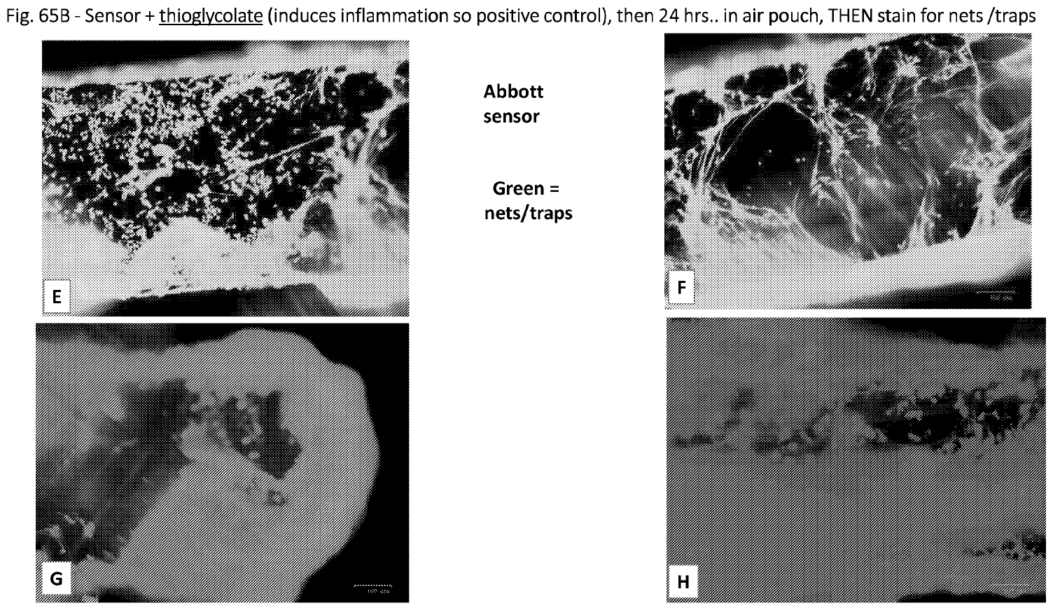

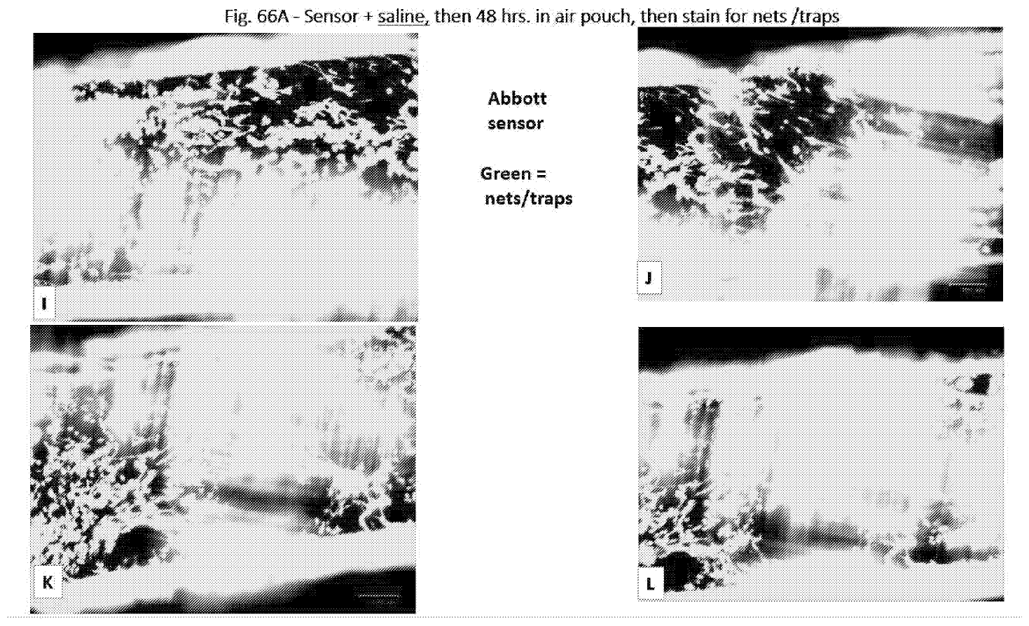

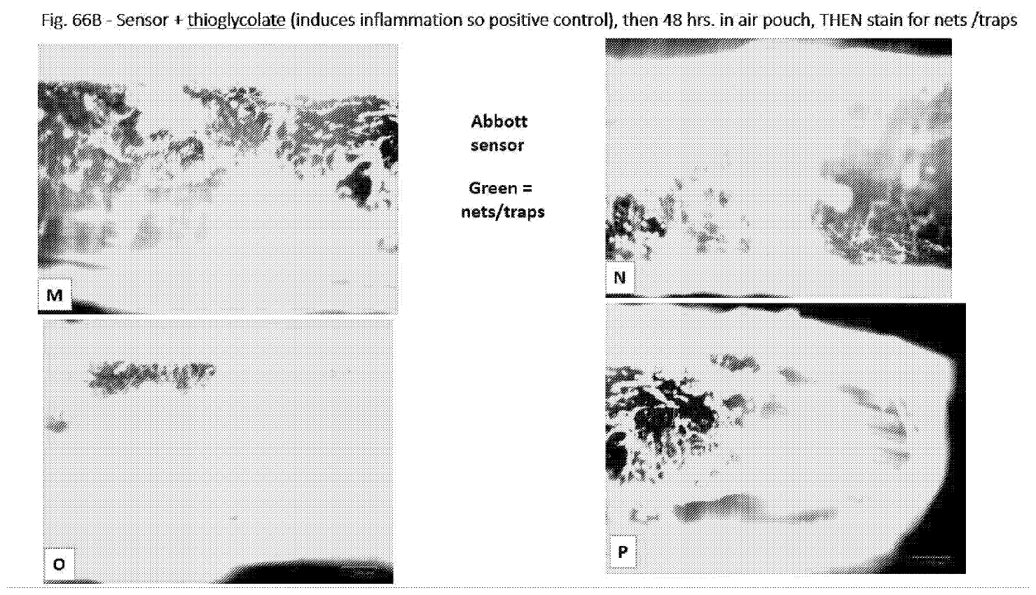

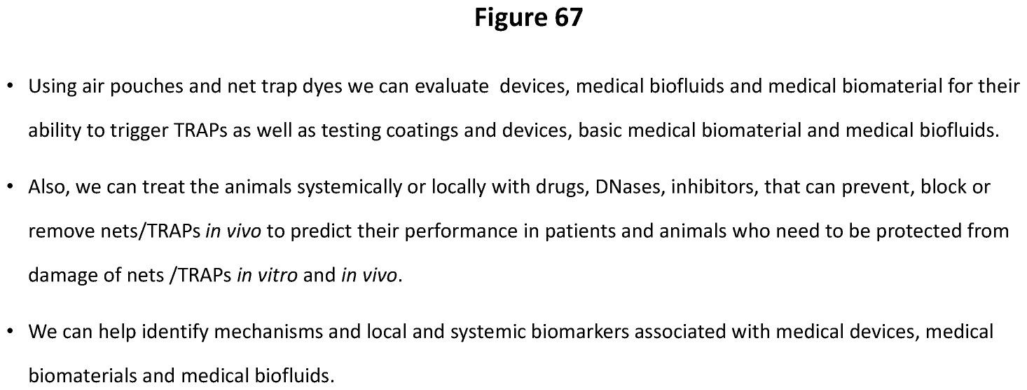

1. Detection, prevention/suppression and/or removal of TRAPs (including NETs), induced directly or indirectly, by implantable medical devices, as well as the development of treatments and agents to prevent and treat acute and chronic diseases/tissue reactions (e.g., inflammation, loss of blood vessel and fibrosis) induced by implantable medical devices TRAPs (e.g. NETs,), (anti-NETs and anti-TRAPs induced diseases). These anti-NET and anti-TRAP treatments and agents can be used for the treatment of acute and chronic disease, including minimizing of symptoms, improve their quality and/or length of life when used in mammals or other living organisms.
2. Detection, prevention and removal of TRAPs (e.g. NETs,), induced directly or indirectly, by non-implantable medical devices, as well as the development of treatments and agents to prevent and treat acute and chronic diseases/tissue reactions (e.g., inflammation, loss of blood vessel and fibrosis) induced by non-implantable medical devices TRAPs (e.g. NETs,), Anti-NET and anti-TRAP treatments and agents can be used for the treatment of acute and chronic disease, including minimizing of symptoms, improve their quality and/or length of life when used in mammals or other living organisms.
3. Detection, prevention and removal of TRAPs (e.g. NETs,), induced directly or indirectly, by medical bio-fluids, as well as the development of treatments and agents to prevent and treat acute and chronic diseases/tissue reactions (e.g., inflammation, loss of blood vessel and fibrosis) induced by medical bio-fluids TRAPs (e.g. NETs,), Anti-NET and anti-TRAP treatments and agents can be used for the treatment of acute and chronic disease, including minimizing of symptoms, improve their quality and or length of life when used in mammals or other living organisms.
4. Detection, prevention and removal of TRAPs (e.g. NETs,), induced directly or indirectly, by medical bio-materials, as well as the development of treatments and agents to prevent and treat acute and chronic diseases/tissue reactions (e.g., inflammation, loss of blood vessel and fibrosis) induced by medical bio-materials TRAPs (e.g. NETs,), anti-NET and anti-TRAP treatments and agents can be used for the treatment of acute and chronic disease, including minimizing of symptoms, improve their quality and/or length of life when used in mammals or other living organisms.
5. Using “Air pouch model” in animals for detection NETS and TRAPS that are induced directly or indirectly by 1) implantable medical devices, non-implantable medical devices, medical bio-fluids, and/or medical bio-materials to determine which of these types of materials or devices can induce TRAPs (e.g. NETs,), in vivo, and use this information to evaluate these devices and material to design materials for uses in medical devices and biofluids, as well as the treatment in patients and animals with TRAP (e.g. NETs,), induced diseases.
6. Using “Air pouch model” in animals to detect and evaluate whether medical devices, medical bio-fluids and or medial biomaterials can induce the formation of TRAPS (nets), or related tissue reactions to screen existing or prototype new medical devices, medical bio-fluids and or medial biomaterials and are coatings (for example see FIGS. 63-67 from insertion of sensor and other medical devices, medical bio-fluids and medical bio-materials into air pouch.)
7. Using “Air pouch model” in animals to detect and evaluate whether medical devices, medical bio-fluids and or medial biomaterials can induce netosis, TRAPS (nets), or related tissue reactions to screen possible treatment for netosis, TRAPs and related tissue reactions induced by medical devices, medical bio-fluids and or medial biomaterials.
8. Using “Air pouch model” in animals to detect and evaluate whether medical devices, medical bio-fluids and or medial biomaterials can induce netosis, TRAPS (nets), or related tissue reactions to screen for in situ or circulating biomarkers that can be used in the detection, diagnosis, treatment of netosis, TRAPs and or related tissue reactions induced by medical devices, medical bio-fluids and or medial biomaterials.
9. Using “Air pouch model” in animals to detect and evaluate anti-NETS and anti-TRAPs agents and treatments that can directly or indirectly prevent and/or treat NET and TRAP induced tissue reactions and diseases induced by 1) implantable medical devices, non-implantable medical devices, medical bio-fluids, and/or medical bio-materials to prevent and/or treat NET and TRAP induced tissue reaction and diseases in patients and animals.
10. Using “Air pouch model” in animals to detect and evaluate anti-netosis, anti-NETs and anti-TRAPs agents and treatments that can directly or indirectly prevent and/or treat NET and TRAP induced tissue reactions and diseases induced by 1) implantable medical devices, non-implantable medical devices, medical bio-fluids, and/or medical bio-materials to screen for new/novel classes of anti-netosis/anti-TRAPs and related tissue reaction treatment and effective drugs and agents to prevent and or reduce the destructive effects on netosis and TRAPs on cells tissues, biological fluids, organs and organisms. In many cases, the anti-TRAP agents are substances or procedures that inhibit, diminish or prevent the formation of TRAPs inside of cells, the release of traps from cells, the cell or tissue toxicity of TRAPs and or TRAP components, TRAP induced injury, TRAP induced inflammation and/or TRAP induced fibrosis.

Use delivery of agents and treatments that can reverse the cell and tissue damage that netosis and TRAPs do to cell and tissue, e.g. agents that induce new blood and lymphatic vessels during both the destructive and reparative phases of tissue response to netosis and TRAPs.

The inventors demonstrate that inhibiting the induction and toxicity of TRAPs prevents or reduces loss of function of medical devices and/or medical bio-fluids associated with implanted devices. This applies to devices and fluid inserted, infused or injected at a tissue site both acutely and in the long term. TRAPs are important to fight off and kill bacteria and viruses, which is clearly helpful in the survival of the infected host. However, netosis and other TRAP formation, even in sterile (non-infectious) tissue injury or even treatment for non-microbial diseases such as treatment of diabetes with insulin, excessive TRAP formation can result in intense and sustained inflammation and fibrosis with loss of vessels, thereby causing permanent loss of the tissue site architecture and function (i.e., fibrosis causes tissue rigidity, and the loss of blood and lymphatic vessels prevents the ingrowth of any cells). The problem of formation of excessive TRAPs at and near the site of an implanted device in contact with tissue, including transdermal tissue and internal tissue, can be addressed by local delivery of netosis and Trap inhibitors and DNase (which dissolves TRAPs) that can prevent/suppress local inflammation/fibrosis/tissue destruction at the implantation site, thereby preventing loss of device and drug/medical bio-fluids function, including insulin formulation function. The embodiments described herein provide for preparing device coatings configured to improve biocompatibility directly by incorporating components directly into the coatings, or by local delivery of netosis/TRAP inhibitors and/or DNase. Additional embodiments described herein provide for biomaterials and fluid that prevent netosis/TRAP.

Disclosed embodiments include medical device and medical bio-material Coatings to Increase Device and medical bio-material Biocompatibility directly or by systemic and or Local Delivery of above Inhibitors/or related agents (e.g. DNase). Other embodiments include medical devices, medical bio-materials/Medical bio-fluids for Implantable and non-implantable Devices that Prevent or reduce or repair destructive Netosis/TRAPs induced reactions. Additional embodiments are methods and devices that minimize tissue damage during insertion, injection and/or infusion of medical bio-materials, medical bio-fluids (example commercial insulin solutions) and/or medical devices that minimize cell death with the formation of tissue cells, leukocyte TRAPs, as well as associated blood clots locally and at distant sites. Furthermore, Netosis biomarkers, including TRAPs and their related products, in blood urine or tissue, or air pouch related tissue cells and fluids, can be used as prognostic, diagnostic evaluation and treatment involving the use of insertion, injection and/or infusion of medical bio-materials, medical bio-fluids (example commercial insulin solutions) and or medical devices to detect, treat or prevent diseases which are directly and indirectly caused by netosis and TRAPs (causal biomarkers) or associated with disease that can trigger netosis and TRAPs as associated biomarkers.

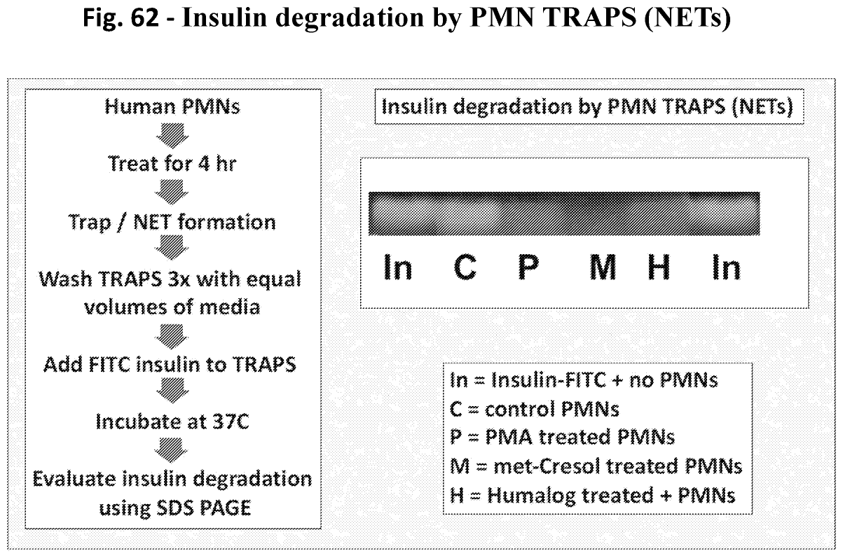

Diabetes—Embodiments disclosed herein solve problems associated with commercial insulin/excipient induced tissue reactions during continuous subcutaneous insulin infusion (CSII) and syringe delivery of insulin. The inventors have found that commercial insulin infusion triggers tissue injury and local inflammatory responses at insulin infusion sites, which ultimately results in infusion site tissue reactions (inflammation, loss of vasculature and fibrosis) resulting in acute and long-term/permanent loss of tissue structure and architecture, resulting in limited infusion site functional longevity, loss of blood glucose regulation due to decreased insulin diffusion and insulin degradations, premature infusion failure and pharmacokinetic (PK) absorption variability. The inventors also have found that commercial insulin phenolic preservative and well as insulin fibrils formation (fibrils are non-functional polymers of the insulin monomers that do not regulate blood glucose levels in vivo) trigger netosis, TRAPs tissue injury and local tissue reactions including inflammatory reactions (inflammation and fibrosis) both during infusion and afterwards (i.e., after cannula withdrawal), that ultimately limit infusion site longevity, loss of blood glucose regulation due to decreased insulin diffusion and insulin degradations, infusion failure and PK absorption. Furthermore, based on the data described herein, the inventors understand that insulin formulations containing phenol and/or m-creosol (excipients/diluents) trigger infusion site netosis and TRAPs formation with tissue injury and local tissue reactions (inflammation and fibrosis), occurring during both infusion and afterwards (i.e., after cannula withdrawal). The consequences of these insulin preservatives, such as phenol and/or m-cresol, induced tissue reactions include limiting infusion site longevity (short and long term), premature infusion failure and pharmacokinetics absorption variability. Based on data, the inventors believe that the influx of chemokine-recruited leukocytes into the infusion site results in the release of leukocyte-derived proteases that degrade insulin. Insulin degradation further limits the effectiveness of insulin mediated blood glucose control in vivo (FIG. 62). The inventors further understand that inhibitors of cytokine, chemokine and leukocyte proteases decreases infusion site inflammation, tissue injury and thereby improve both short-term (decrease inflammation) and long-term (decrease fibrosis) continuous subcutaneous insulin infusion including insulin injection performance and blood glucose control in vivo.

The inventors have also demonstrated in vitro (TRAP formation in cell culture, and in vivo (air pouch assay) that the insulin infusion cannula can trigger netosis and TRAP formation both in vitro and in vivo.

One embodiment described herein uses an anti-TRAP agent to reduce inflammation at an insulin infusion site. Non-limiting examples of anti-TRAP/anti-TRAP agents that can be used in the disclosed embodiments are deoxyribonuclease (DNase), ribonuclease (RNase), an inhibitor of peptidyl arginine deiminase 4 (PAD4 inhibitor), a histone-degrading enzyme, an antibody against a component of an extracellular TRAP, an inhibitor of chromatin recondensation, and plasmin.

Another embodiment uses an anti-TRAP agent to reduce inflammation, vessel loss and fibrosis at the site of contact between a patient's cells and tissue and an implantable or non-implantable device, medial biofluid or a medical bio-material.

A further embodiment uses an anti-TRAP agent to reduce inflammation vessel loss and fibrosis in a patient resulting from the use of a medical bio-fluid such as an infused drug, for example, insulin.

Yet another embodiment is a screening tool or biomarker used to determine whether a medical device or a medial biomaterial, or medical bio-fluids is likely to cause netosis and TRAP formation with associated tissue reactions of inflammation, loss of blood vessels and fibrosis in a patient. The screening tool or biomarker can be used for in vitro, ex vivo or in vivo testing.

In embodiments, the anti-TRAP agent is incorporated into a medical bio-fluids or a coating of a medical device (e.g. sensors, cannula, surgical meshes) or medical bio-materials. In embodiments, the anti-TRAP agent is supported by a biological or synthetic matrix. In some cases, the anti-trap agent is incorporated into a liposome or particle, which may be a nanoparticulate material used directly as a coating or the nanoparticles can be incorporated into nature or synthetic coatings or polymers or viscous oils. In some cases, the anti-netosis and or anti-TRAP agent is included in a coating on an implantable or non-implantable medical device.

Non-limiting examples of natural and synthetic matrices to support the anti-TRAP agents are described below:

### Pharmaceutically Acceptable Carriers—Non-Limiting Examples of Natural & Synthetic Matrices:

Natural Matrices: Basement membrane (BM) is a highly biocompatible coating for implantable medical devices. Basement membrane can be directly coated to the outer surface of an implantable device, as a single layer or multiple layers of different combinations of basement membrane (BM) or hydrogels (HG)) as medical device coatings.

Crosslinking of BM with low levels of glutaraldehyde prior to insertion into tissue dramatically extends the BM and medical device lifespan in vivo, without loss of biocompatibility. Additionally, the inventors have found that natural crosslinking agents (i.e., genipin) can also effectively crosslink BM without loss of biocompatibility of the BM.

Synthetic Matrices: Non-animal/human protein coatings also can be used. Non-limiting examples of suitable synthetic matrices are synthetic matrices for stem cells grown in vitro/in vivo (Sigma TruGel3D). TruGel3D has 9 synthetic matrices used for in vitro & in vivo growth of stem cells. The inventors believe these synthetic matrices are suitable for use with anti-TRAP agents. As shown below, these matrices have a polyvinyl alcohol or dextran backbone polymer, a non-cell-degradable crosslinker or cell-degradable crosslinker, such as polyethylene glycol, and bioactive materials, such as RGD peptide. Arginylglycylaspartic acid (RGD) is the most common peptide motif responsible for cell adhesion to the extracellular matrix (ECM), found in species ranging from Drosophila to humans.

### Anti-TRAP Agents

Non-limiting examples of anti-TRAP agents are materials that degrade, or target for degradation, a component of a TRAP, inhibit the activity of a TRAP or TRAP component, and/or prevents the formation of a TRAP. According to US Published Patent Application No. 2021/0023183, for example, anti-NET agents include nucleic acids—DNA, RNA, small molecules, lipid, carbohydrate, protein, peptide, antibody, or antibody fragment and DNases. These materials also inhibit the formation of various types of TRAPs in addition to NETs released from neutrophils.

In some embodiments, an anti-TRAP compound can be an enzyme, e.g. an enzyme which cleaves and/or degrades, e.g. a nucleic acid, protein, polypeptide, or carbohydrate. As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (i.e., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

According to US Published patent application No. 2021/0023183, an anti-NET compound can be, but is not limited to; DNase, RNase, heparin, an antibody (i.e. an antibody to histones or to a particular histone), a histone degrading enzyme (i.e. mast cell proteinase 1 (Gene ID:1215)), plasmin (Gene ID: 5340), cathepsin D (Gene ID:1509) or activated protein C (Gene ID:5624)) or an inhibitor of chromatin recondensation (i.e. staurosporine, HDAC inhibitors (i.e. M344), PAD4 inhibitors, or elastase inhibitors (i.e. Gelin®)). And a wide variety of chemicals and other agents. The inventors believe many of these compounds also inhibit TRAPs from various cell types induced by medical devices, medical bio-materials, medical bio-fluids, and Tissue Implantation induced trauma induced traps.

Anti-TRAP agents include therapeutic RNAs and RNAs non-coding RNAs, such are microRNAs (miR), modified messenger RNA (Med-mRNA), small RNAs, circular RNAs, etc., which can functional to prevent, suppress netosis, TRAP formation and degradation, control these therapeutic RNAs and RNAs non-coding RNAs, can act both as anti-netosis and anti-TRAPs agents, as well as inhibit netosis and TRAP induced tissue reactions e.g., prevent inflammation, loss of vasculature and fibrosis, as well as promote repair of injured cells and tissue such as inducing new blood and lymphatic vessel in injured tissue to prevent fibrosis and promote cell survival and proliferation. Also modified mRNA can produce additional inhibitors on destructive components present on TRAPS. For example, because TRAP related proteases (e.g. elastase) can destroy not only tissues, but also insulin itself using modified mRNA that transfects cells and induce extended expression of protease inhibitors such as alpha-i-anti-trypsin protein, this would prevent tissue and insulin degradation and commercial insulin infusion sites. This would not only save tissue sites for future implantation and insulin infusion, but it would decrease the amount of insulin needed to regulate blood glucose levels in patients with diabetes, extend the function lifespan of the insulin infusion site, as well as decreases the cost of insulin needed to regulate blood glucose levels in patients with diabetes. It should be noted that currently, insulin cost for patients without insurance can run for $100-$1000 of dollars a month. Proteases are anti-TRAP agents that neutralize the effect of TRAPS.

Anti-protease such as those that inhibit elastase and other proteases include alpha 1 anti-trypsin, and aprotinin (see Table A).

TABLE A
Other examples of TRAP inhibitors are shown in the figures.

| Drug | Inhibits | Manufacturer IC-50 | | Range | |
|---|---|---|---|---|---|
| DPI | NADPH Oxidase | 50 nM & 0.3 μM | | 10 μM, 32 μM | |
| 4-ABAH | MPO | 0.3 | μM | 100 | μM |
| GW311616A | Neutrophil | 22 | nM | 20 | μM |
| | Elastase | | | | |
| CI-Amidine | PAD | 0.8, 6.2, 5.9 | μM | 200 | μM |
| Go6976 | PKC | 7.9, 2.3, 6.2 | nM | 100 | nM |

| | | | | |
|---|---|---|---|---|
| Nocodazole | Microtubule | 0.2, 0.53, 0.64 | μM | 2.5 μM, 10 μM |

Additionally, Cayman Chemical, Selleck, Sigma-Aldrich and others sell compounds that are TRAP inhibitors.

TABLE B
The following are non-limiting examples of commercially
available TRAP inhibitors Table B.

1 μM Wortmannin (PI3K inhibitor; Sigma-Aldrich),
1 μM R406 (SYK inhibitor; Selleckchem),
10 μM SB203580 (p38 MAPK inhibitor; Selleckchem), 1
0 μM SB202190 (p38 MAPK inhibitor; Santa Cruz Biotechnology) 1
0 μM MLN0128 (pan-mTORC inhibitor; Selleckchem),
10 μM BAPTA-AM (chelator of Ca2+; Focus Biomolecules),
1 μM DPI (NADPH oxidase inhibitor; Sigma-Aldrich),
1 μM Rapamycin (mTORC1 inhibitor; Selleckchem),
1 μM Celastrol (NF-κB inhibitor; InvivoGen),
0.1 μM Bay-11-7082 (IκB phosphorylation inhibitor; InvivoGen),
0.1 μM GW5074 (cRaf1 kinase inhibitor; Santa Cruz Biotechnology),
0.1 μM U-73122 hydrate (PLC inhibitor; Sigma-Aldrich),
1 nM Bafilomycin A (vacuolar-type H+ -ATPase inhibitor;
Sigma-Aldrich),
0.1 μM JNKi II 420128 (JNK inhibitor; Merck Millipore) or
0.1 μM Mytoquinone (mitochondrial ROS inhibitor; BIOTREND)
10 μM 4-ABAH (MPO Inhibitor, Cayman)
100 μM GW311616A (Neutrophil Elastase Inhibitor, Adipogen)
20 μM Cl-Amidine (PAD Inhibitor, Cayman)
100 nM Go6976 (PKC Inhibitor, Selleck)
10 μM Nocodazole (Microtubule Inhibitor, Stem Cell)
10 μM 6-gingerol (Selleck)
10 μM 8-gingerol (Cayman)
100 μM 10-gingerol (Cayman)

### Other Anti-TRAP Agents Include DNases Such as DNase-1.

Additional anti-TRAP agents include protease inhibitors such Elastase inhibitors including natural inhibitors such as alpha 1 antitrypsin or synthetic inhibitors.

US Patent Publication No. 2016/0067315 describes a lactoferrin compound that can be used in combination with a pharmacologically acceptable carrier.

Another known anti-TRAP agent is JAK1/2 inhibitor.

Yet another known anti-TRAP agent is the 21-mer autophagy regulator peptide P140. Extracellular TRAPs formed by neutrophils have been studied. Other types of cells also form extracellular TRAPs, including but not limited to macrophages and epithelial cells (see FIG. 63 for additional examples), including endothelial cells. Other DNA-containing cells also have the potential to form TRAPs.

TABLE C
Examples of major mammalian cell types cells

| | |
|---|---|
| Stem cells | Embryonic stem |
| | cells |
| | Adult stem cells |
| White blood cells | Granulocytes |
| | (neutrophils, |
| | eosinophils, |
| | basophils) |
| | Agranulocytes |
| | (monocytes, |
| | lymphocytes) |
| Nerve cells | Neurons |
| | Neuroglial cells |
| Muscle cells | Skeletal |
| | Cardiac |
| | Smooth |
| Cartillage cells | Chondrocytes |
| Bone cells | Osteoblasts |
| | Osteoclasts |
| | Osteocytes |
| | Lining cells |
| Skin cells | Keratinocytes |
| | Melanocytes |
| | Merkel cells |
| | Langerhans cells |
| Fibroblasts | Fibroblasts |
| Endothe lial | Lining blood |
| | vessels |
| Epithelial cells | Lining body cavities |
| Fat cells | White adipocytes |
| | Brown adipocytes |
| Sex cells | Spermatozoa |
| | Ova |

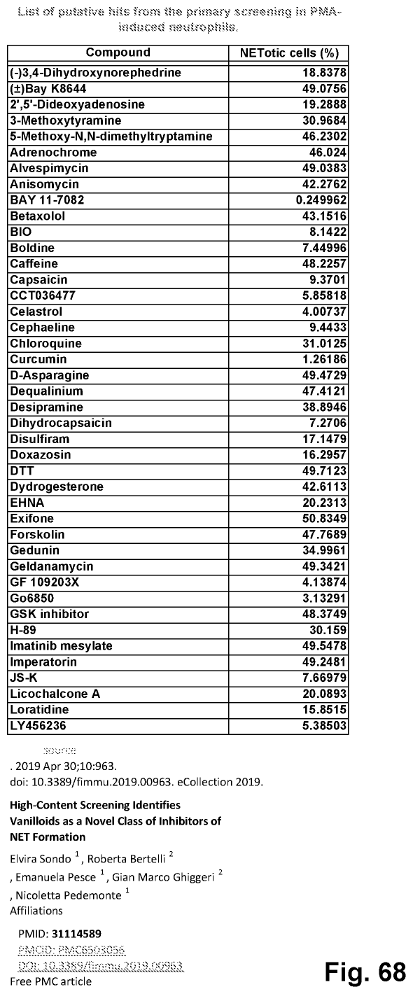

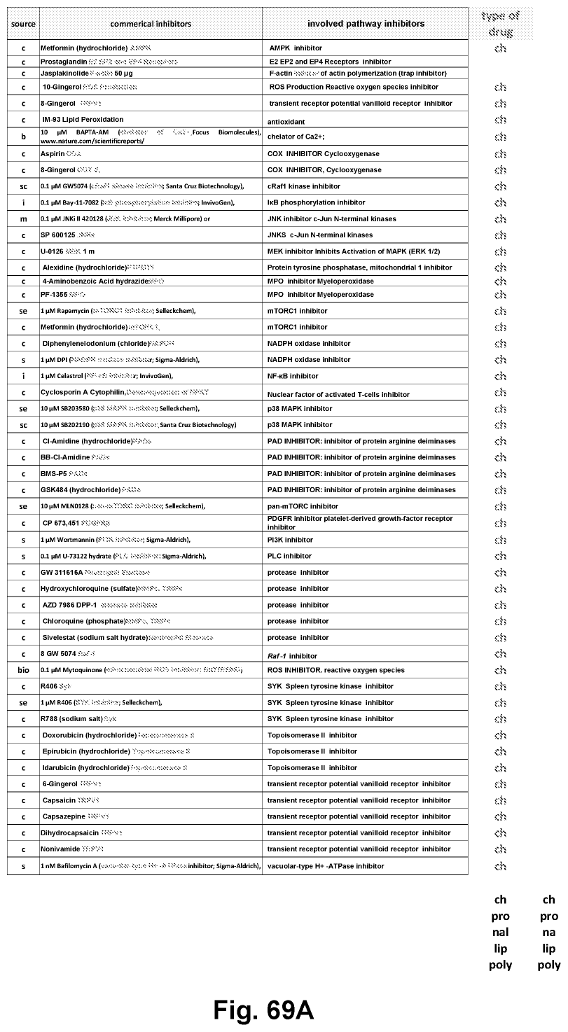

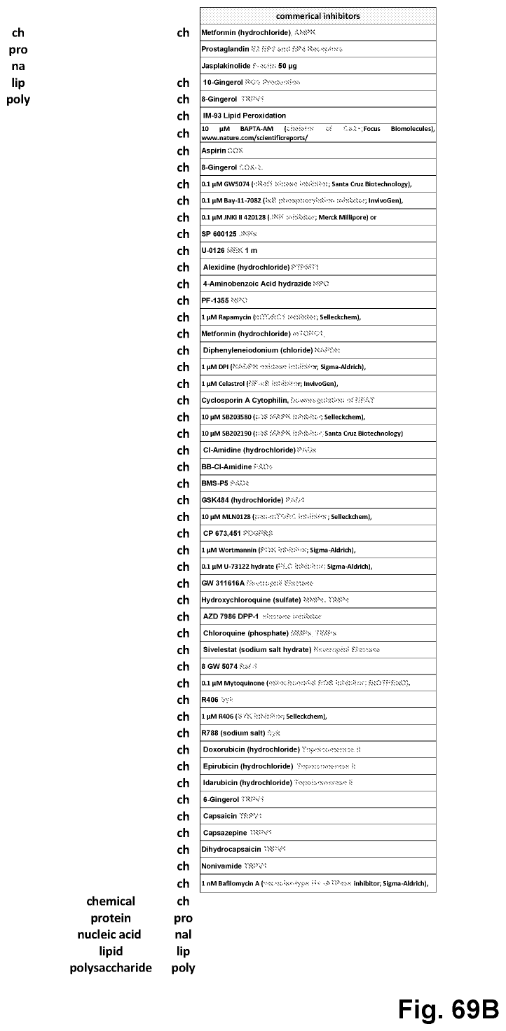

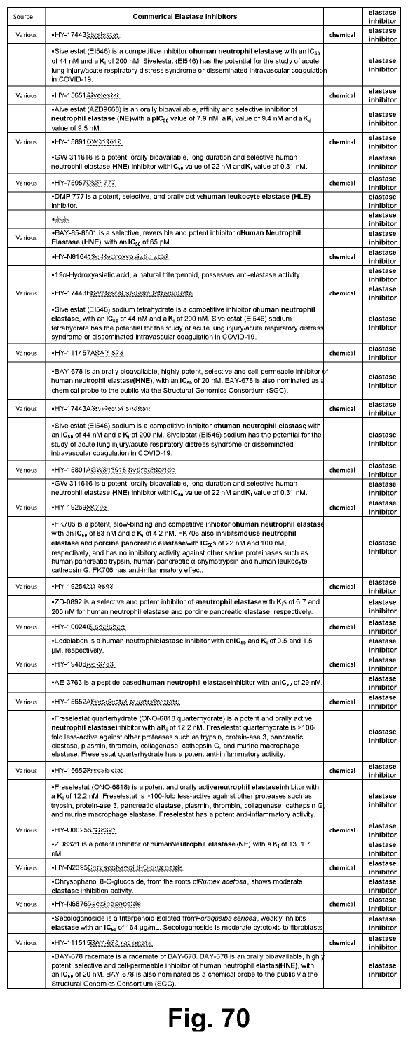

FIGS. 68-71 list additional inhibitors that are anti-TRAP agents.
Table D provides examples of nucleated cells in the human body that can undergo netosis and release of TRAPs which include leukocytes, bone marrow cells, endothelial cells, epithelial cells, neurologic cells, stem cells, embryonic cells, fibroblasts and all other DNA nucleated cells in mammals:

TABLE D
Examples of percent major cell types

| Cell type | % cell count |
|---|---|
| Bone marrow cells | 2.5 |
| Vascular endothelial cells | 2.1 |
| Lymphocytes | 1.5 |
| Hepatocytes | 0.8 |
| Neurons and glia | 0.6 |
| Bronchial endothelial cells | 0.5 |
| Epidermal cells | 0.5 |
| Respiratory interstitial cells | 0.5 |
| Adipocytes (fat cells) | 0.2 |
| Dermal fibroblasts | 0.1 |
| Muscle cells | 0.001 |
| Other cells | 2.0 |

### DESCRIPTIONS OF EMBODIMENTS AND DATA

FIG. 1A summarizes the role of medical devices, medical bio-materials, medical bio-fluids, and tissue implantation trauma inducing TRAP formation, and inflammation at implantation sites in accordance with embodiments described herein. FIG. 1B schematically illustrates TRAPs. TRAPs are DNA-based structures, that are released from cells with a nucleoles, which are dying and undergoing netosis. For example, macrophages produce TRAPs (MQ-TRAPs), endothelial cells produce TRAPs (E-TRAPs), and mast cells produce TRAPs (MC-TRAP). NETs are TRAPs that are released from neutrophils that are dying and undergoing netosis. Thus, all NETs are TRAPs, but not all TRAPs are NETs.

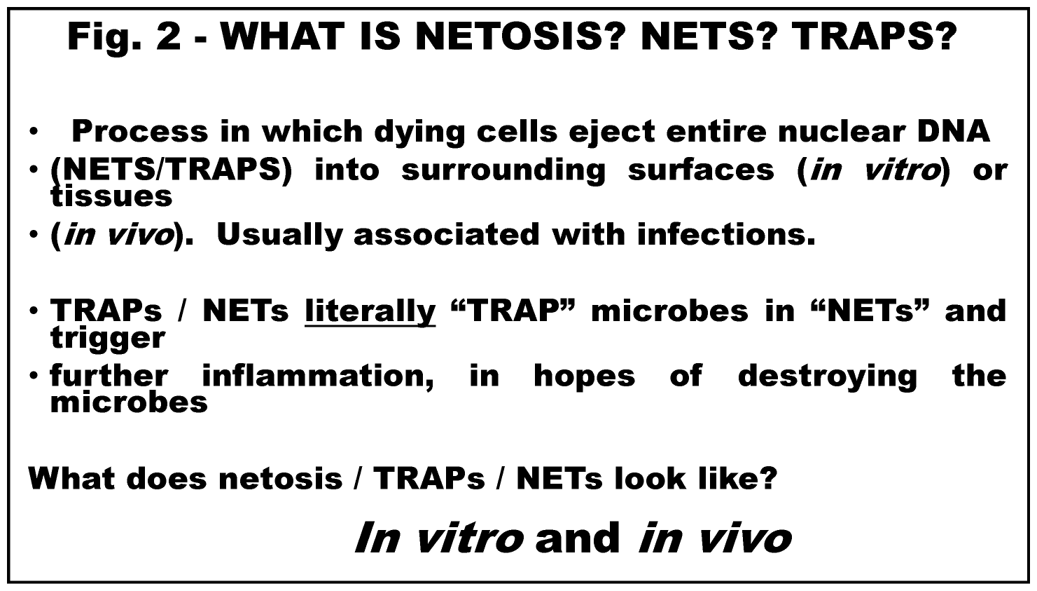

FIG. 2 describes netosis and TRAPs. Originally, netosis and TRAPS were associated with microbes, and their products. The inventors in this application believe that TRAPS all cells (including NETS (TRAPS from neutrophil) can be triggered by medical devices, medical bio-materials, medical bio-fluids, and tissue implantation.

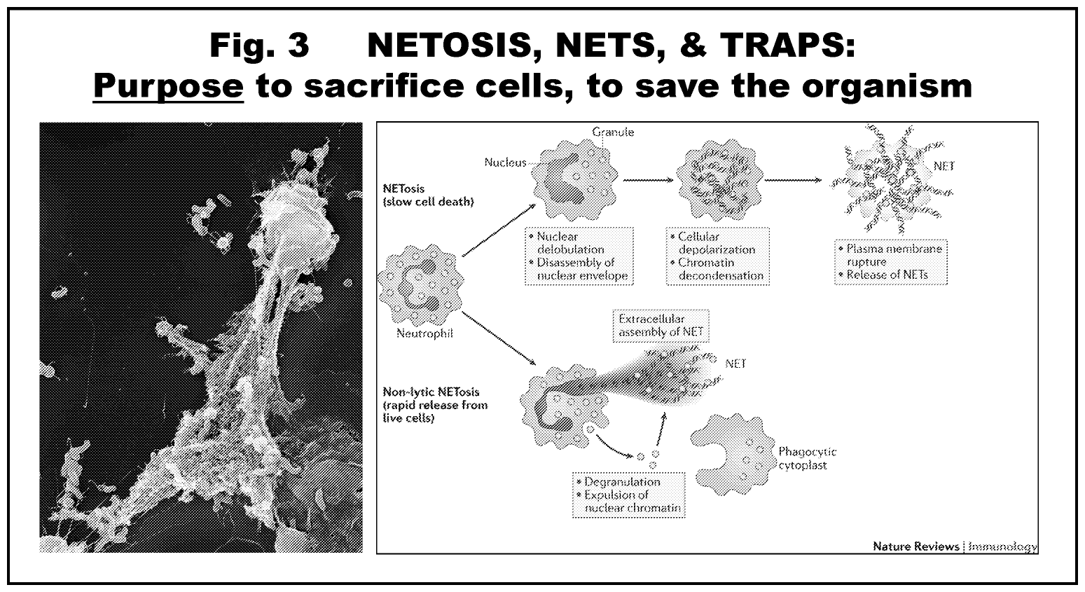

FIG. 3 schematically shows the processes of netosis (slow cell death) and non-lytic netosis (rapid/explosive release of TRAPs from dying cells). Both Netosis and non-lytic netosis results in cell death and the release of TRAPs, with destructive outcomes. Non-lytic netosis results in degranulation and the expulsion of nuclear chromatin, with residual phagocytic cytoplast with short term metabolic (e.g., oxygen radicals), proteolytic activity (e.g., elastase) and synthetic capability (e.g. protein synthesis).

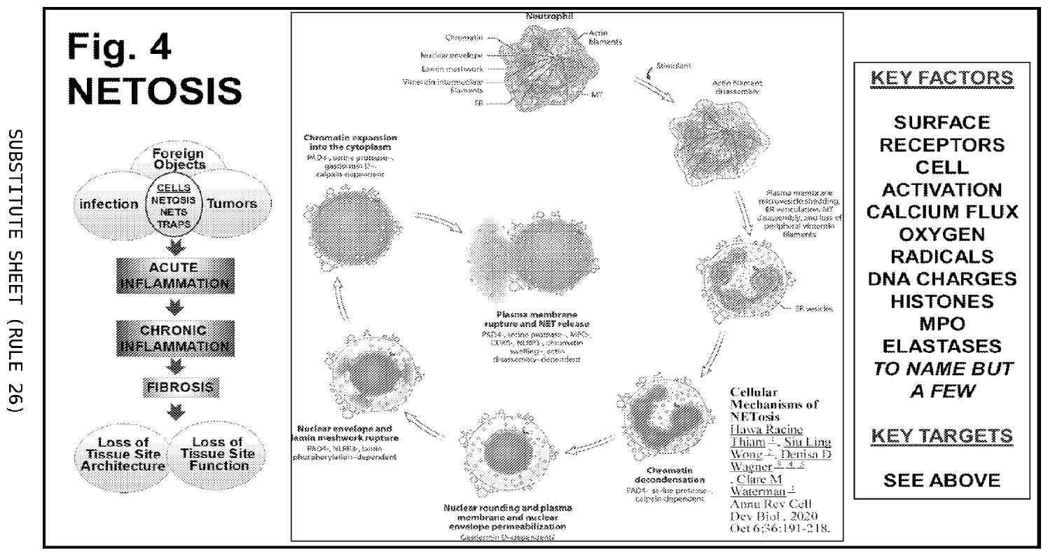

FIG. 4 includes a schematic flow chart showing that the presence of foreign objects in tissues can lead to infection, cell netosis, and the formation of TRAPs. These cause acute inflammation as well as chronic inflammation, loss of vasculature, which lead to fibrosis. Fibrosis results in the loss of tissue site architecture and/or loss of tissue site function. When an implant is present, such as a glucose sensor, this can lead to the loss of sensor function. Key factors in netosis and TRAP formation, as well as triggering of tissue reactions include surface receptors, cell activation, calcium flux, oxygen radicals, DNA charges, histones, myeloperoxidase (MPO), elastases, cytokines, etc. to name but a few, also see FIG. 5. Key targets include the same.

FIG. 5 schematically depicts triggers for TRAP formation, and activation of the formation of TRAPs.

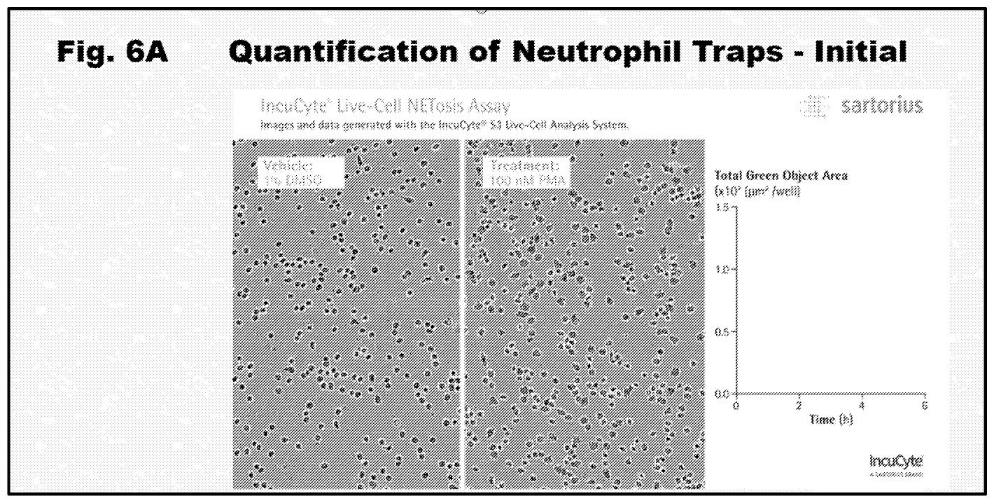

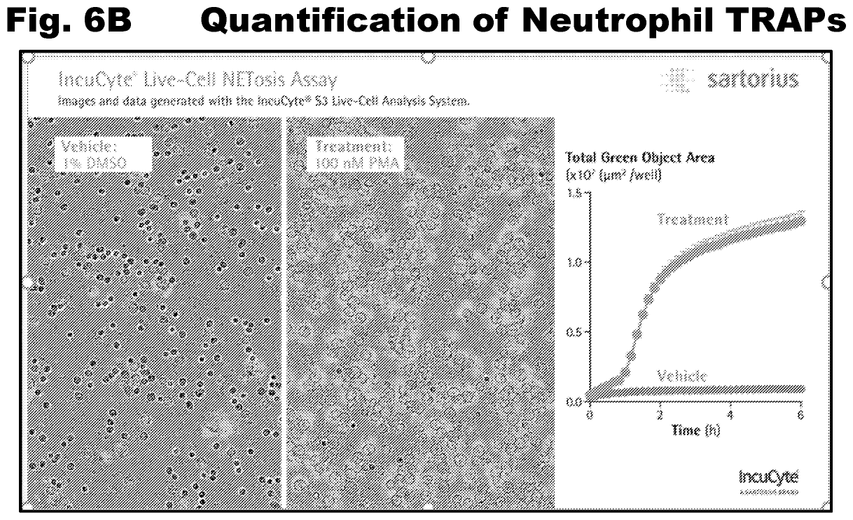

FIG. 6A-6B show a technique for quantifying TRAPs.

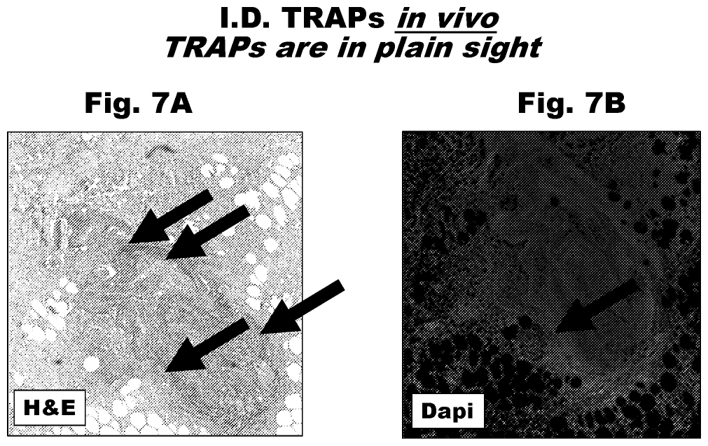

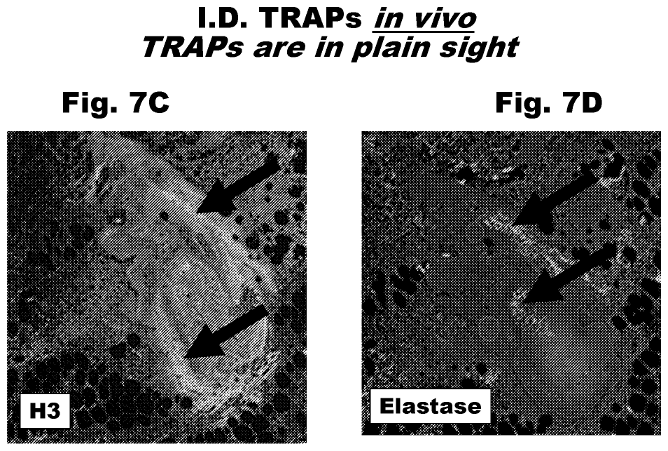

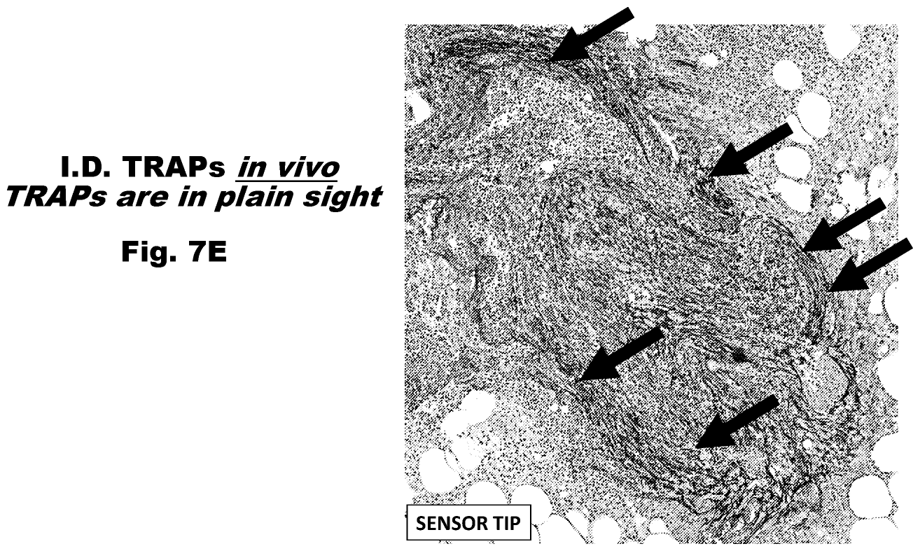

FIG. 7 shows a series of histological swine biopsy sections with the presence of TRAPs at site of an implanted glucose sensor. In FIG. 7, black arrows indicate the presence of TRAPs directly, e.g., extracellular DNA using a hematoxylin stain (A and E) or DAPI (B) staining. The presence of TRAPS are also detected as TRAP bound markers, such as fluorescent antibodies to Citrullinated H3 histones (C), as well as fluorescent antibodies to elastase (D). Most importantly H&E images show consistent purple web staining appearance in tissue sections (A) and (D). These studies demonstrate that implantation of glucose sensors trigger TRAP formation in the skin of swine and likely contribute to inflammation and scarring, which limits sensor function over time.

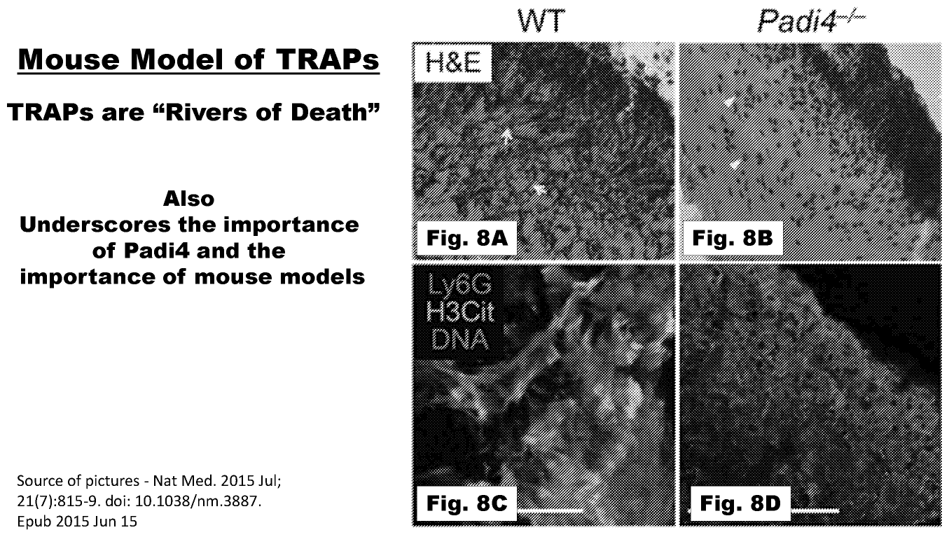

In FIG. 8, the images are from the cited reference, which demonstrates that mice deficient in Padi4 protein inhibit TRAP formation in skin wounds but appear in the skin of wild type (WT) mice. In the case of WT mice TRAPS are detected in H&E-stained tissue section, while citrated histones, neutrophil are detected with lyg6 antibody positive and DNA with DAPI. Padi4^{-/-} mice do not show TRAP formation following skin injury. Left-hand side demonstrates the use of the term “Rivers of death” by cells that have been triggered to release their DNA to the extracellular space of forming TRAPs, rivers appear (dark blue to) black in pictures. H&E images show consistent purple web staining appearance in tissue sections (A) and (D). The presence of TRAPs are also detected as TRAP bound markers, such as fluorescent antibodies to Citrullinated H3 histones (C), as well as fluorescent antibodies to elastase (D). These figures also show the importance of Padi4 and the swine model.

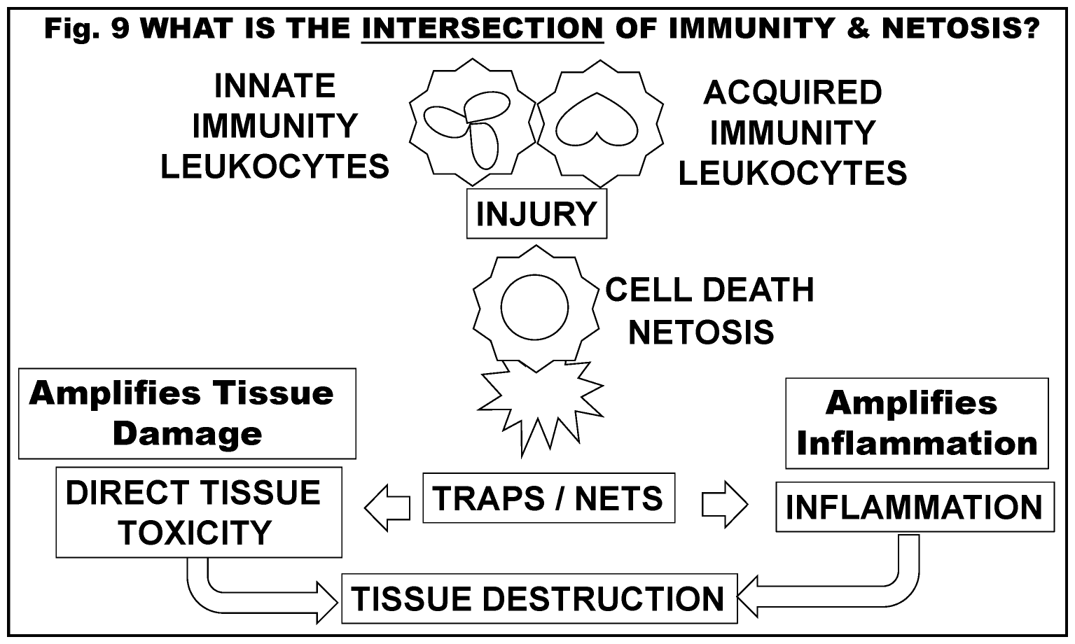

FIG. 9 schematically shows the intersection of immunity and netosis. The formation of TRAPs amplifies tissue damage due to direct tissue toxicity and amplifies inflammation, both of which lead to tissue destruction. FIG. 9 demonstrates the inventor's understanding of the overlap of immunity and NETOSIS that trigger TRAPS induced by implantable devices, medical fluids or other biomaterials or medical devices. Subsequent trigger of inflammation and associated wound healing results in destruction of the tissue side that results in loss of tissue architecture and function, e.g., loss of blood vessels and nerves.

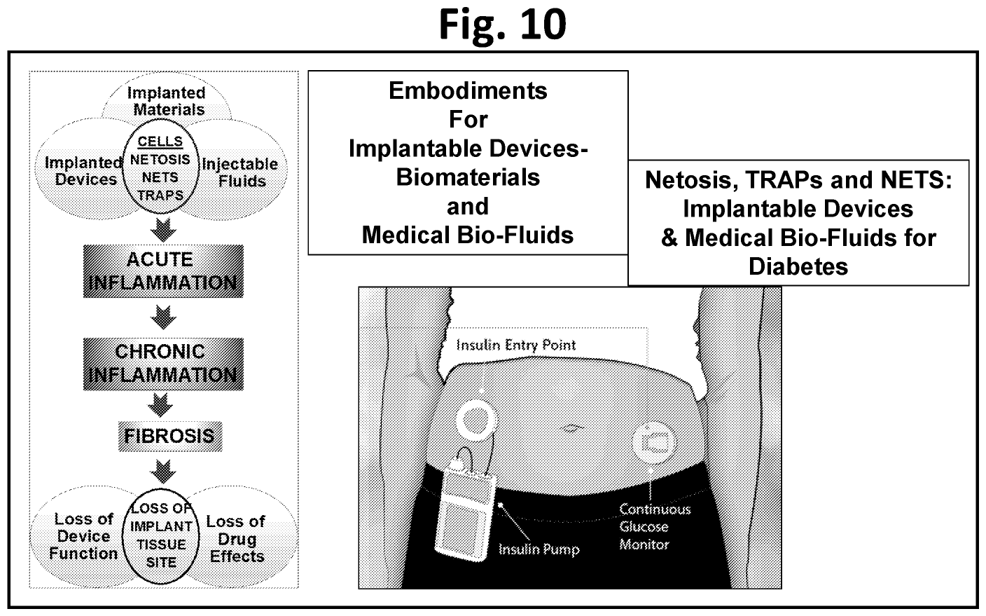

FIG. 10 shows the use of anti-TRAP and anti-TRAP agent to improve the function, especially the lifespan, of an insulin pump cannula. The figure contains original subject matter in combination with an illustration from the internet showing the presence of both an implantable glucose sensor and insulin infusion set on the body to show where they are located when they are used in patients with diabetes or other disease using these types of devices.

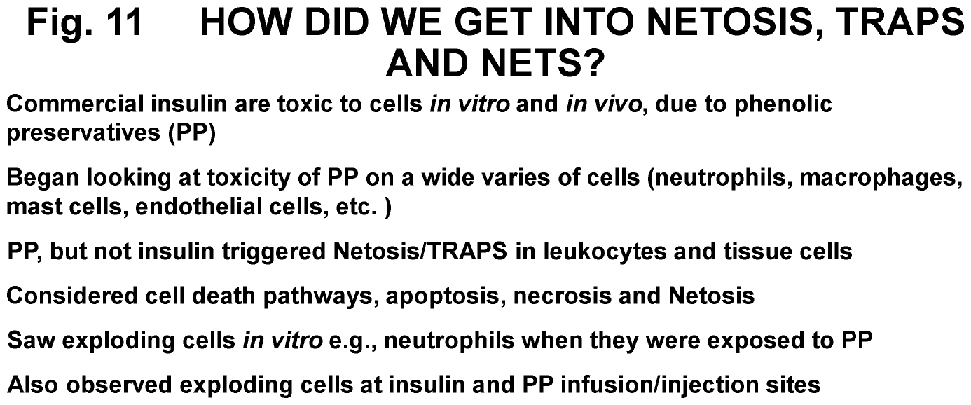

FIG. 11 lays out the history of the inventor's detection of TRAPS in vitro using commercial insulin containing phenolic preservatives, including phenols and m-cresol, as well as the detection of TRAPS in the skins of experimental animals, such as mice and pigs.

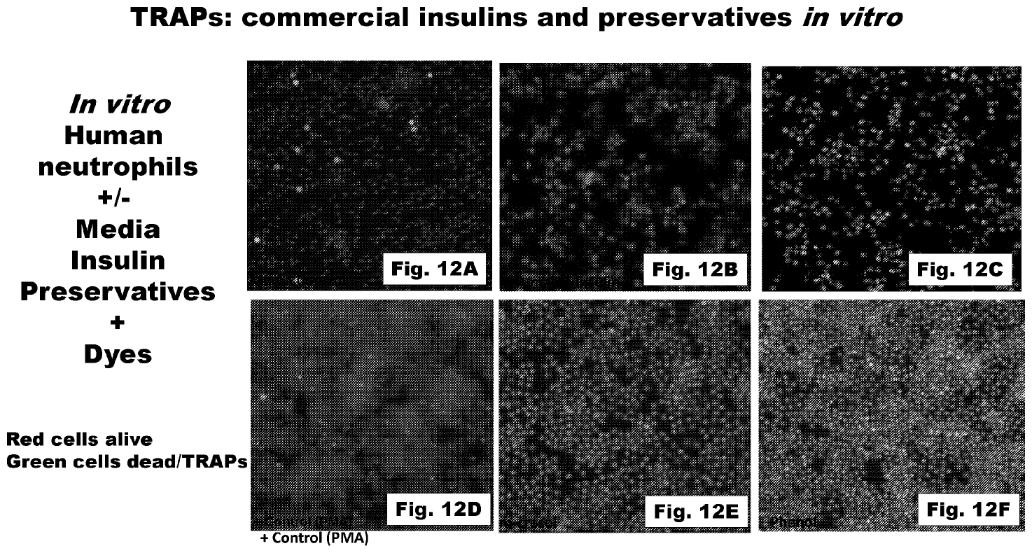

FIG. 12 demonstrates the results of in vitro studies in which we cultured human blood neutrophils with various agents to determine the ability of commercial insulin and related phenolic preservatives, used in commercial insulin formulation, to trigger TRAPS in human neutrophils media. From left to right and top to bottom (A)-(F), media only (A) and PMA (B), a known inducer of TRAPS in human neutrophils, present positive control. Commercial Humalog insulin (B) and the generic insulin version (C) trigger TRAP formation in human neutrophils as does the addition of phenolic preservatives, such as m-cresol (E) and phenol (F). This is the first demonstration of the ability of commercial insulin and phenolic preservatives to trigger TRAP formation in human neutrophils in vitro.

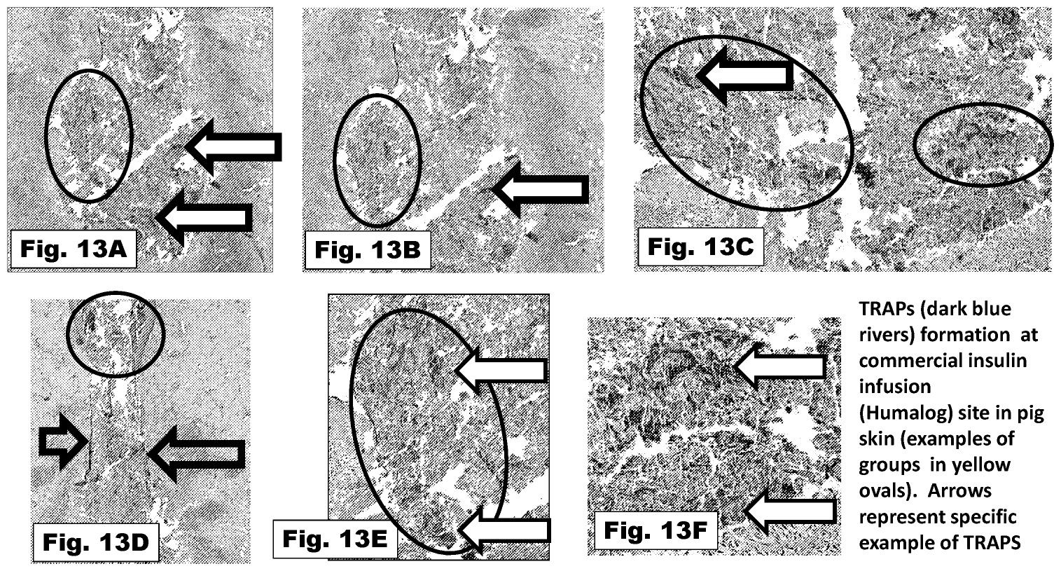

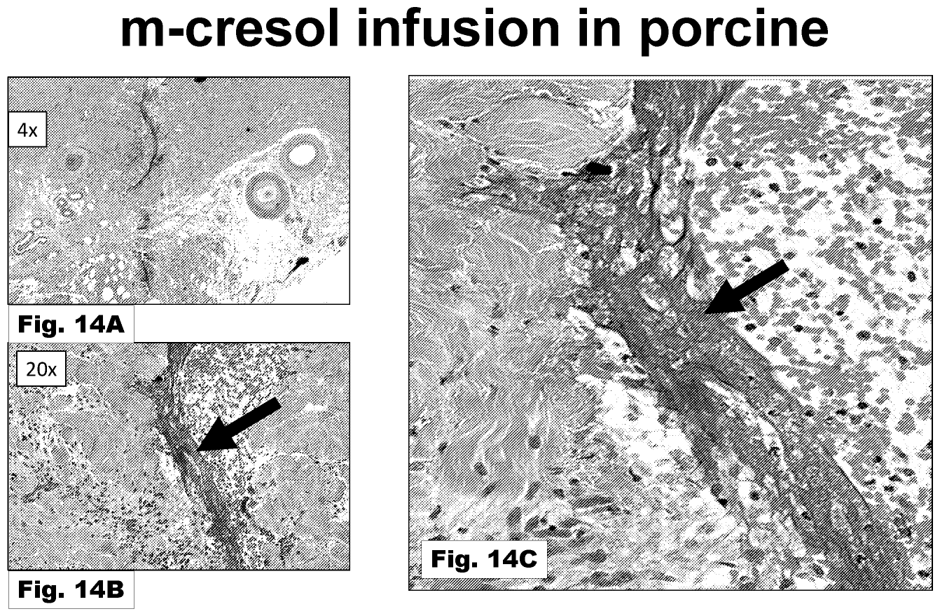

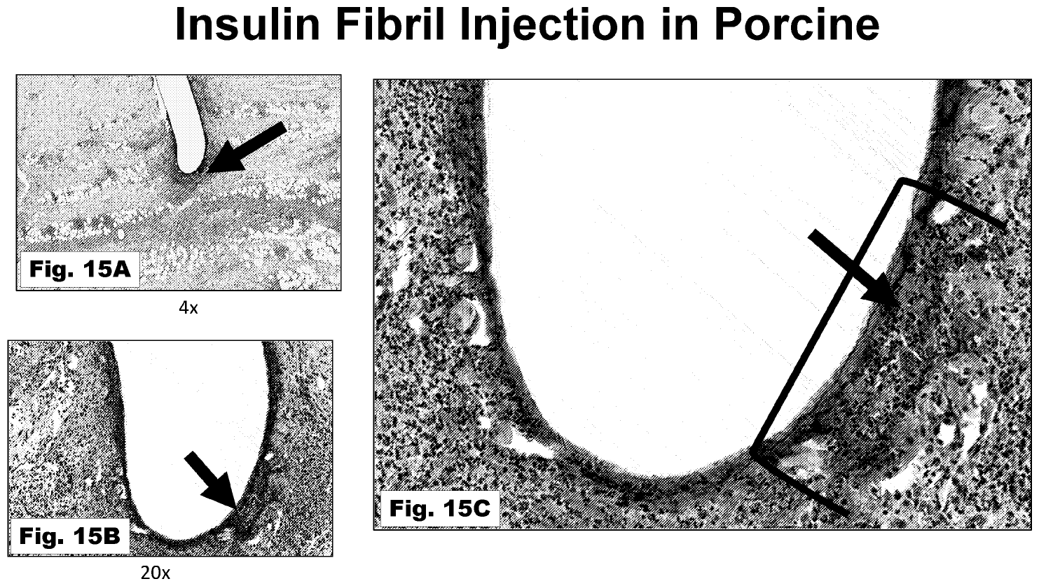

FIG. 13 shows histology of commercial insulin induced TRAP formation in vivo in pigs.

FIG. 14 is a histologic section using H&E staining. It demonstrates the presence of TRAPS in swine tissue infused continuously with m-creosol over a 5- to 7-day period. M-cresol is one of the main preservatives in commercial insulin formulations, such as Humalog. The presence of the purple web appearance is indicated by the black arrows. These studies demonstrate that preservatives present in commercial insulin can trigger TRAPS in vivo when infused into the skin.

FIG. 15 demonstrates insulin derived insulin fibrils injected in porcine skin trigger inflammation, as well as TRAP formation. Insulin fibrils are a large oligomer characterized by a non-native D-sheet structure rendering the protein inactive. The appearance of TRAPS is seen by the dark purple web-like formation at the injection port material tissue interface. Areas of TRAP presence are indicated by the black arrows in the image. White area in the center of the image is the previous location of the injection port.

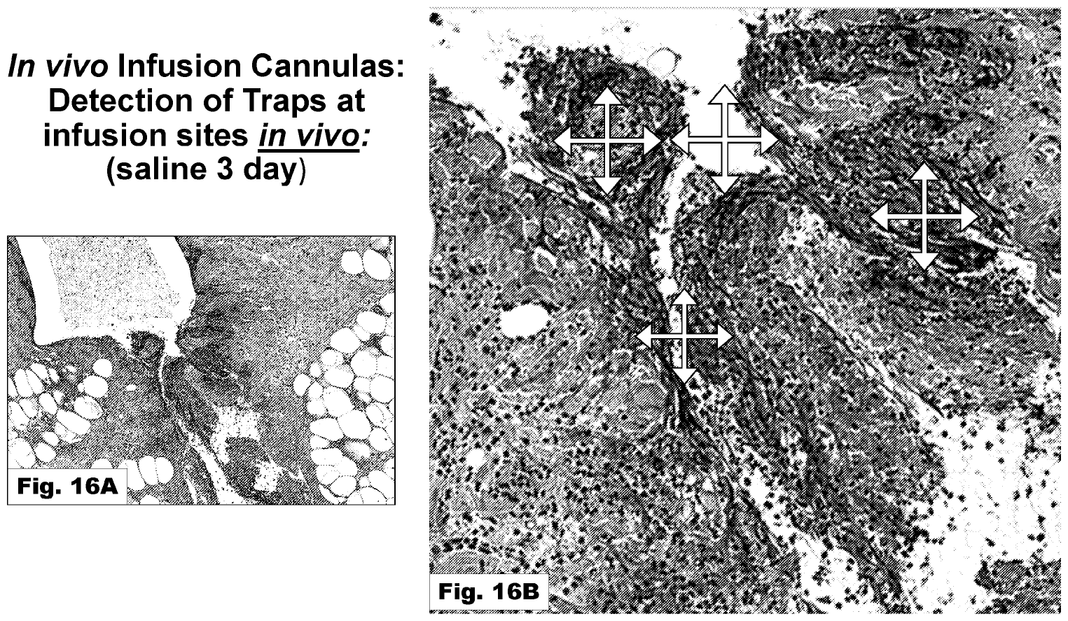

FIG. 16 is a histologic section of swine skin that was infused with saline using a cannula and infusion pump identical to that used for insulin infusion. As can be seen in this H&E tissue section is inflammation and TRAP formation as a result of cannula insertion, continuous saline infusion and the presence of the catheter (A). Presence of TRAPS as indicated by the white arrows in (B). This data demonstrates that in addition to the ability of phenolic preservatives to trigger inflammation and TRAPs, the insertion of the cannula (insertion trauma), as well as possible micro-movement of the cannula overtime trigger inflammation and TRAP formation. This information and TRAP formation is likely initiated due to the insertion of the cannula into the skin which triggers both edema and mass cell activation which begins the inflammatory process, as well as formation of TRAPS at the insertion site, e.g., insertion trauma as well as foreign body reactions triggered by the chemistry of the cannula itself.

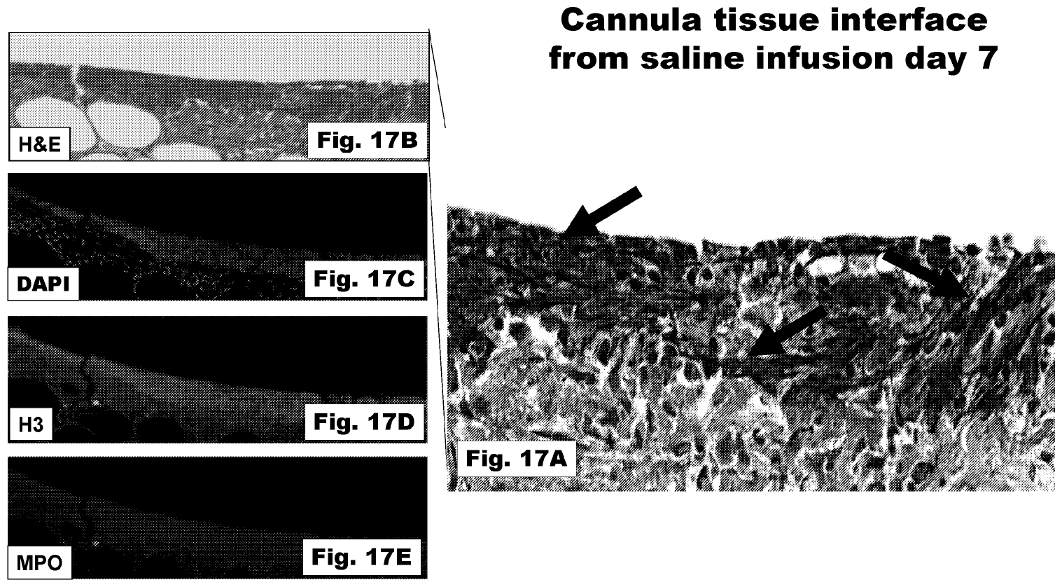

FIG. 17 contains histologic sections from the pig tissue adjacent to cannula. The cannula infused saline for 7 days, followed by tissue biopsy, sectioning and staining (H&E right side). This slide demonstrates that the interaction of the plastic cannula and the tissue results in inflammation and the formation of TRAPs. The TRAPs are represented by the black arrows pointing towards the surface towards the tissue of the section the white space above the tissue section the location of the cannula prior to sectioning because the cannulas fallout once their section is cut, similar to a knot hole that appears when the knot falls out of cut lumber. The sections on the left confirm the presence of the TRAPs as indicated by the arrows. This slide supports the hypothesis that medical devices like cannula can directly trigger TRAPS in vivo.

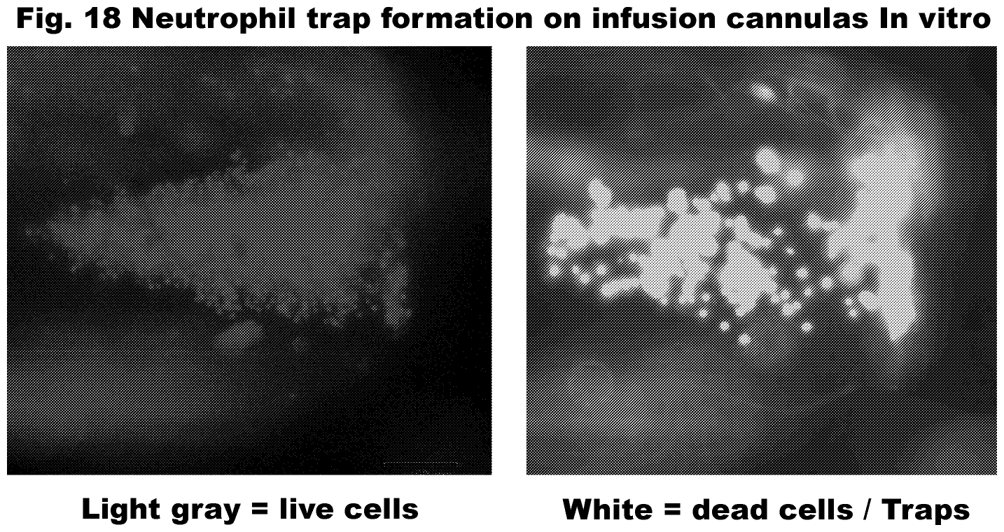

FIG. 18 is an in vitro study, in which the cannula (identical to those in the slide 32 above) are cultured in vitro with human neutrophils. At various time points, dyes are added to detect both live cells (red) or dead cells with TRAPS (green in color, white in grayscale)) when viewed under a microscope. What can be seen in the left-hand side is the beginning of the study the cells the neutrophils are all gray (alive). With time in contact with the hard plastic cannula, the neutrophils rapidly die and trigger the release of TRAPs (white portion in right side image). This slide supports the hypothesis that in fact, biomaterial used in implantable devices and materials, such as those used in cannulas, can directly trigger TRAP formation by neutrophils in vitro and supports our in vivo observation that the cannula biomaterials are responsible for TRAPS seen in vivo studies in FIG. 17.

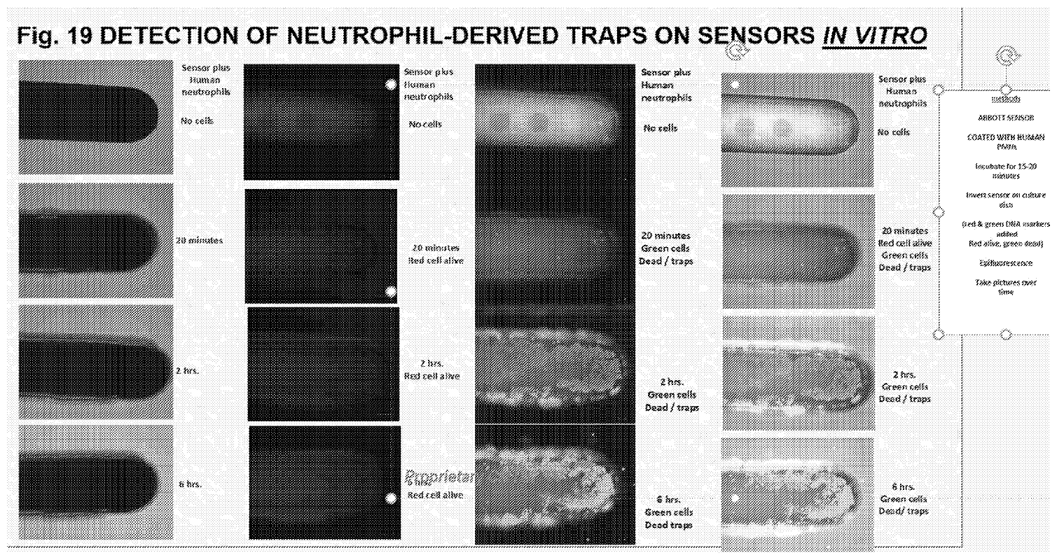

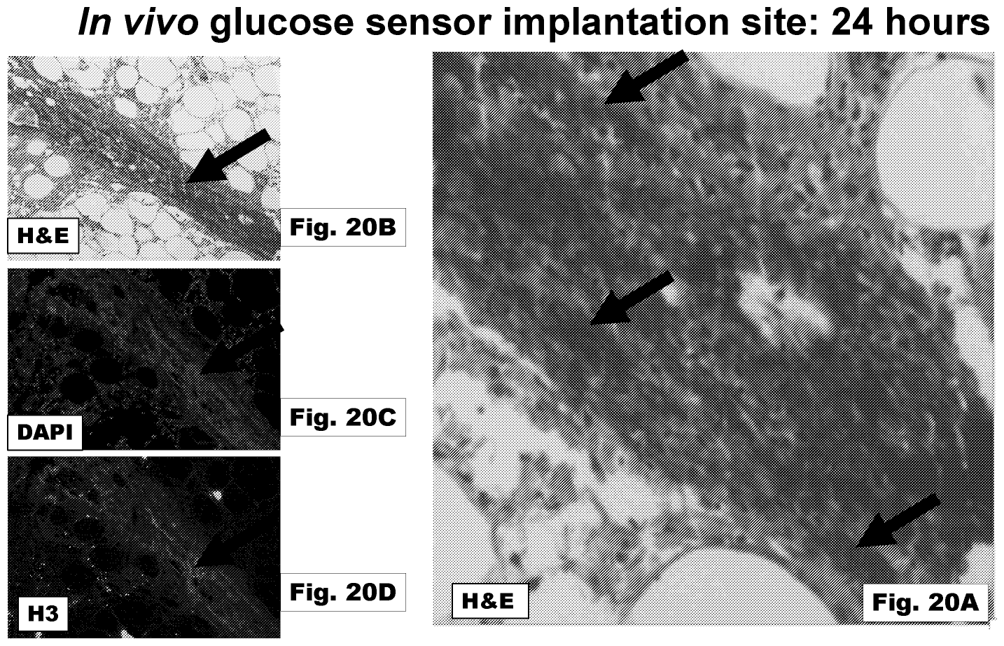

FIG. 19 is similar to the general approach describe for cannulas above, but it is focused on the ability of glucose sensors when cultured with human neutrophils in vitro to trigger neutrophil cell death and the generation of the TRAPS. The top row is no cell and sensor only showing autofluorescence. The next rows are images at various timepoints+/−dyes. Far right row is composite of the left images. Left column: brightfield image of sensor (black/grey); Second column from left shows red (light) staining indicating live cells; Third column from left: green (light colored) staining indicates dead cells; last column from left: is the composites of the live and dead cell staining to the left of this image.

As was the case with the cannulas, what we see here is that if neutrophils are exposed to glucose sensors in vitro, the sensors trigger cell death with the production of TRAPS. This confirms our in vivo data, and it underscores how in vitro test using cells and dyes can predict the potential in vivo biocompatibility of devices, materials as well as biomedical fluids like insulin and preservatives; e.g., if it triggers substantial amount of TRAPS in vitro it is probably not going to work very well in people.

FIG. 20 is an image of a histologic section from swine skin biopsy, in which the commercial glucose sensor Libre from Abbott Diabetes Care was implanted over 24-hours. The biopsy was processed and stained with H&E and evaluated for TRAP presence. As can be seen in the picture on the right-hand side, intensive TRAP formation is associated at sites of sensor implantation. This demonstrates an association between TRAPS, and neutrophils at sites of sensor implantation. The various stains on the left-hand side are using the same battery of antibodies and dies to prove that the TRAPS are present in the swine skin and associated with the hematoxylin (H&E) staining.

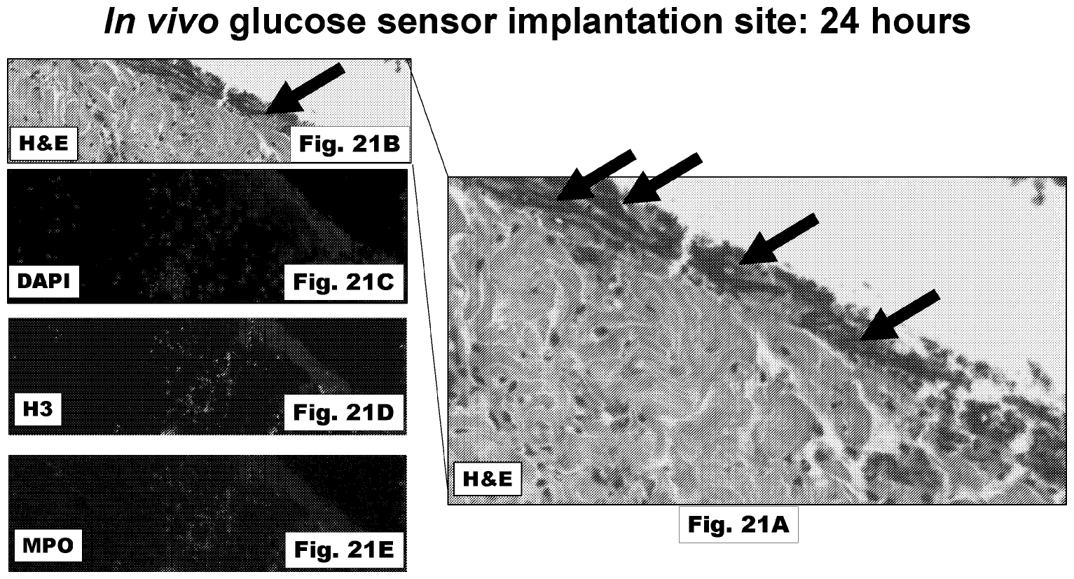

FIG. 21 contains histologic sections derived from swine skin biopsy of a FreeStyle Libre sensor implanted over a 24-hour time period. Right side image shows the TRAP formation at the tissue and glucose sensor interface. The white (empty) area at the tissue sensor interface pertains to the previous sensor location. The special fluorescence staining on the left-hand side are the standard markers to confirm the presence of TRAPS at the sensor tissue interface.

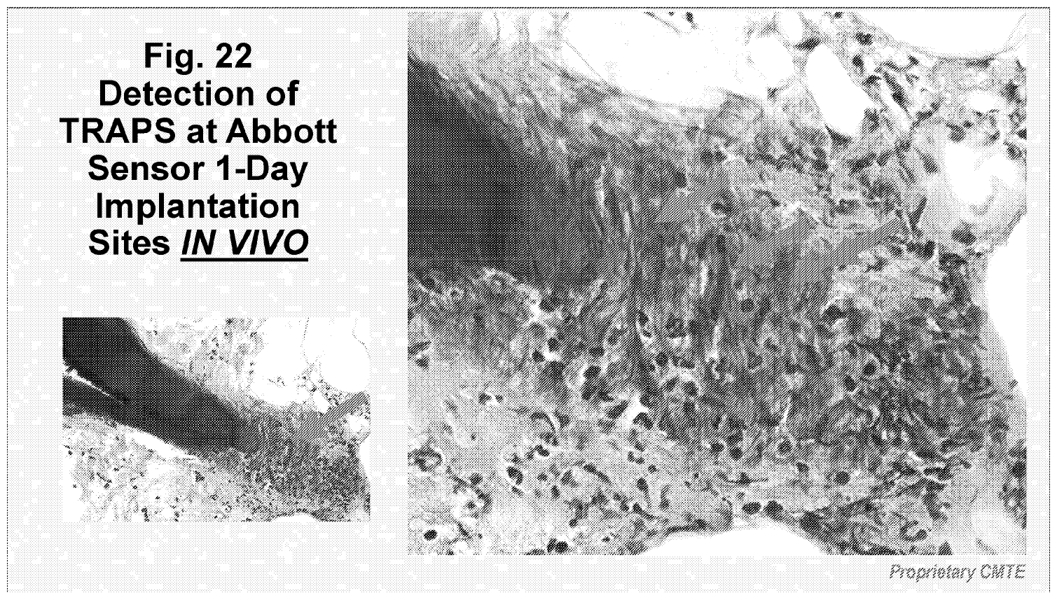

FIG. 22 is another tissue section stained with H&E and demonstrates TRAPS presence at the interface between the tissue and the FreeStyle Libre glucose sensor. In the slide the sensor coating is still within the tissue as seen by the dark black cylinder on the left-hand side of the images. Larger image on the right-hand side clearly see the sensor projecting out of the tissue on the left as well as the development of TRAPS (red arrows) on the right hand side. Red (gray) arrows indicate examples of TRAPS. This additional data shows consistent presence of TRAPS at sensor implementation sites as soon as 24 hours after implantation.

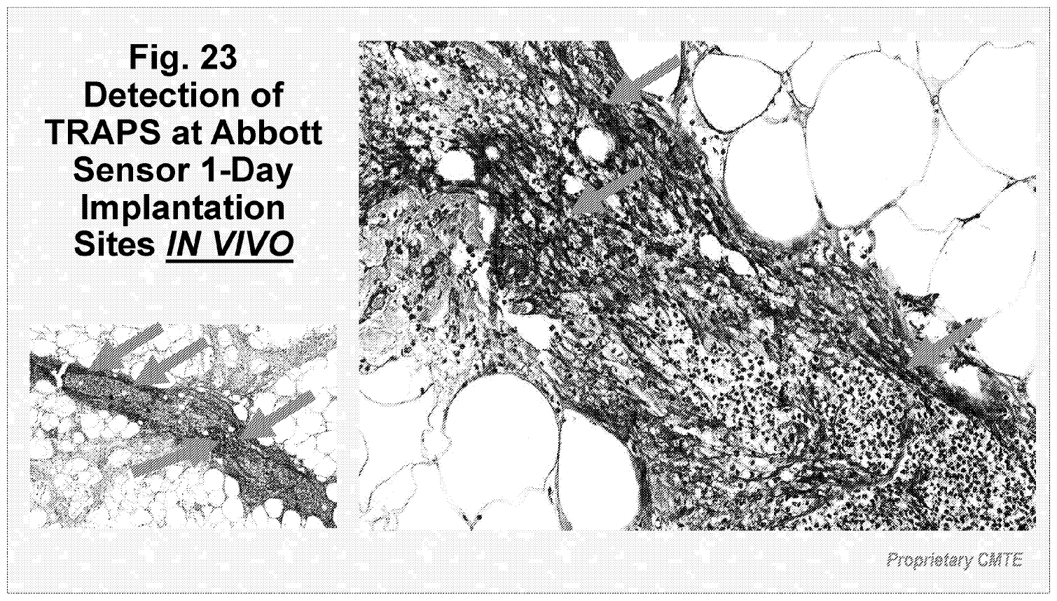

FIG. 23 is another FreeStyle Libre example implanted in swine skin with TRAPS forming around sensors and as a result of the device insertion impact.

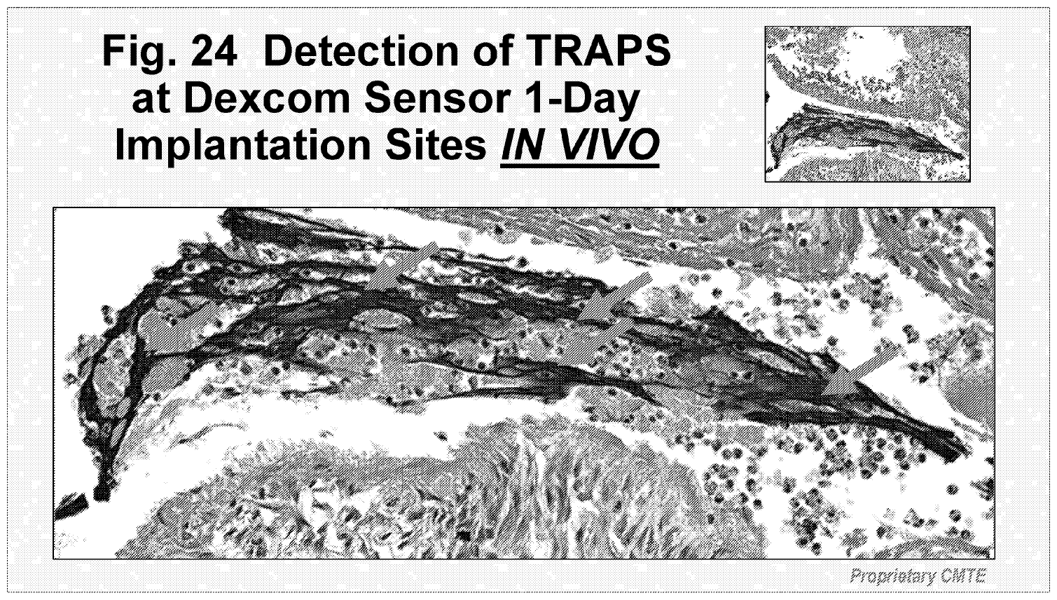

FIG. 24 is additional examples of Dexcom sensors in pig skin with TRAPs forming around sensors.

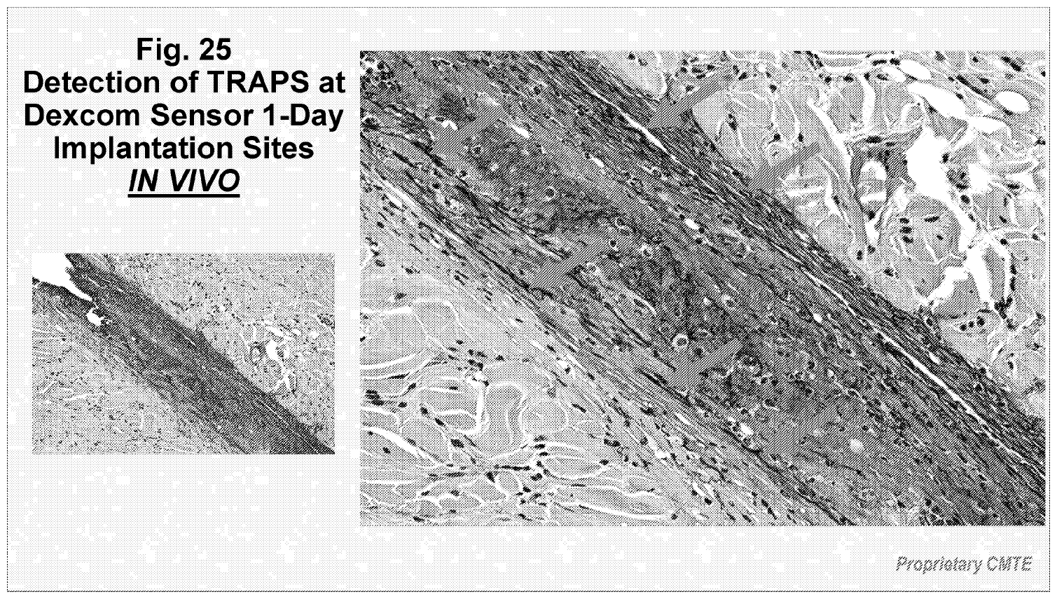

FIG. 25 shows additional examples of Dexcom sensors in pig skin with TRAPs forming around sensors.

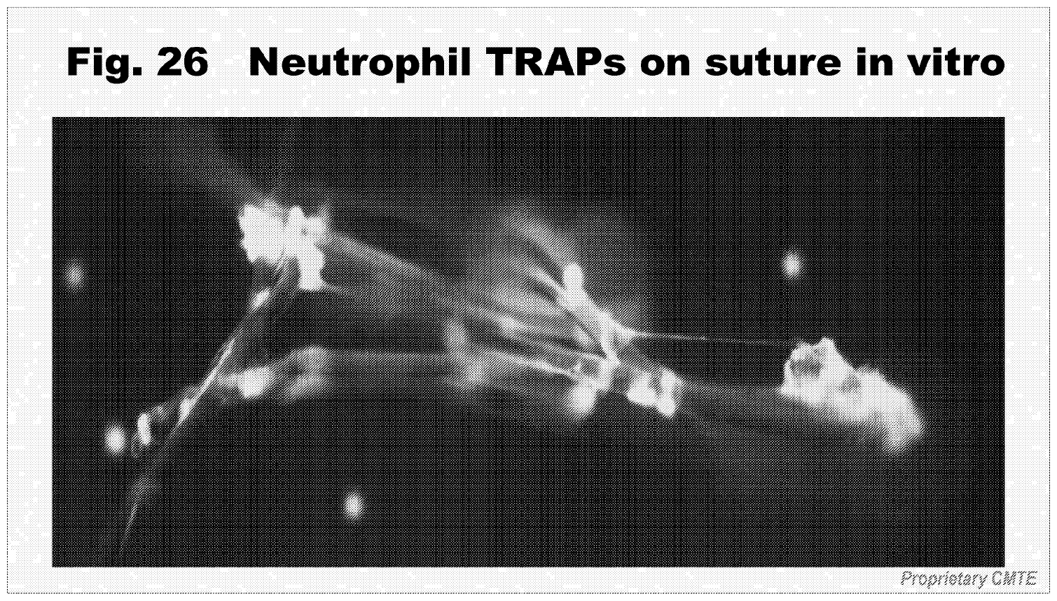

FIG. 26 is an example of another implantable device, i.e., surgical sutures. When surgical sutures are exposed to human neutrophils in tissue culture in vitro, TRAPS are triggered indicated by the green fluorescence staining of TRAPS as well as extra cellular DNA released by the dying/dead neutrophils.

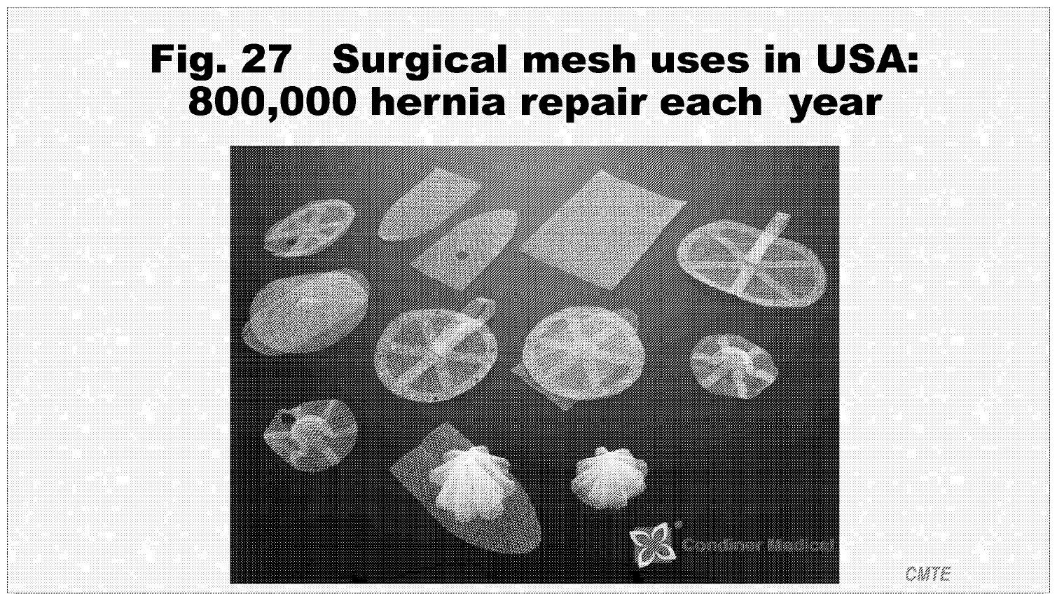

FIG. 27 shows commercially available mesh.

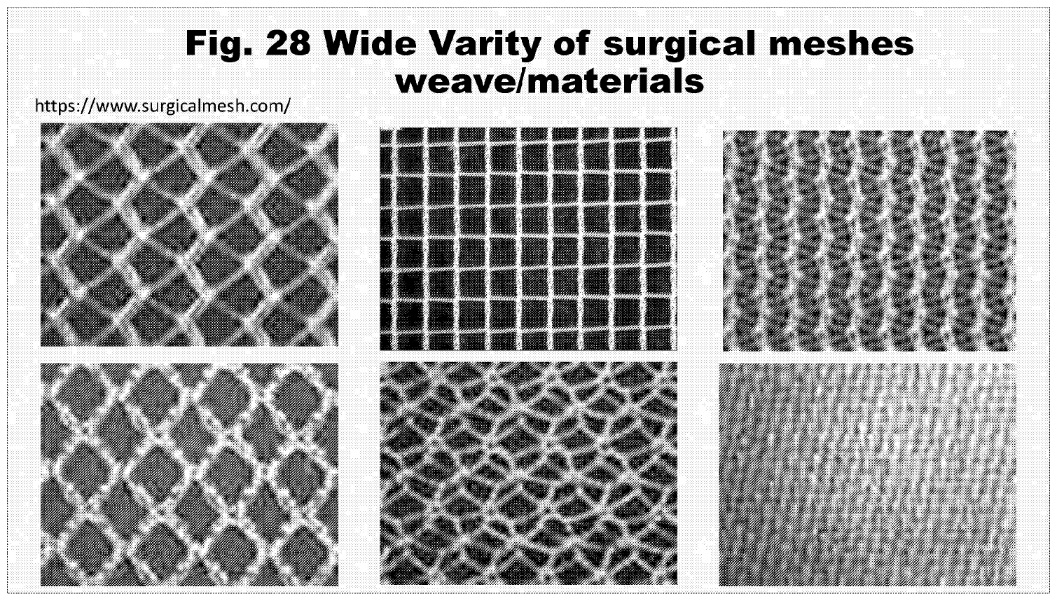

FIG. 28 provides commercial mesh source example of the different meshes the inventors used in studies.

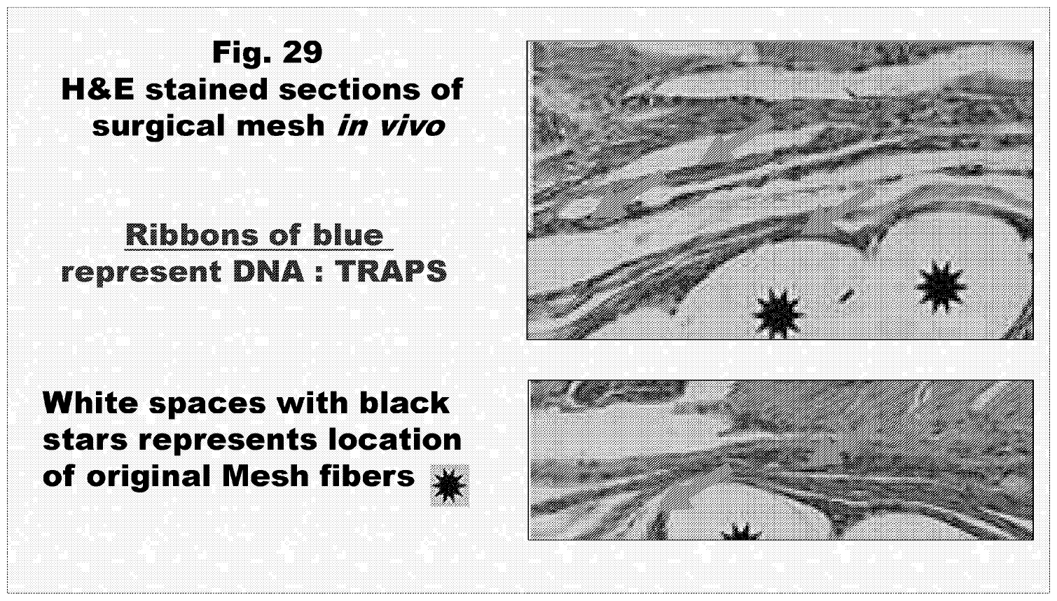

FIG. 29 is a histologic section of the mouse abdomen biopsy in which surgical mesh was implanted and then removed, fixed and stained with hematoxylin (H&E). The image demonstrates the presence of TRAPS indicated by the red (gray) arrows.

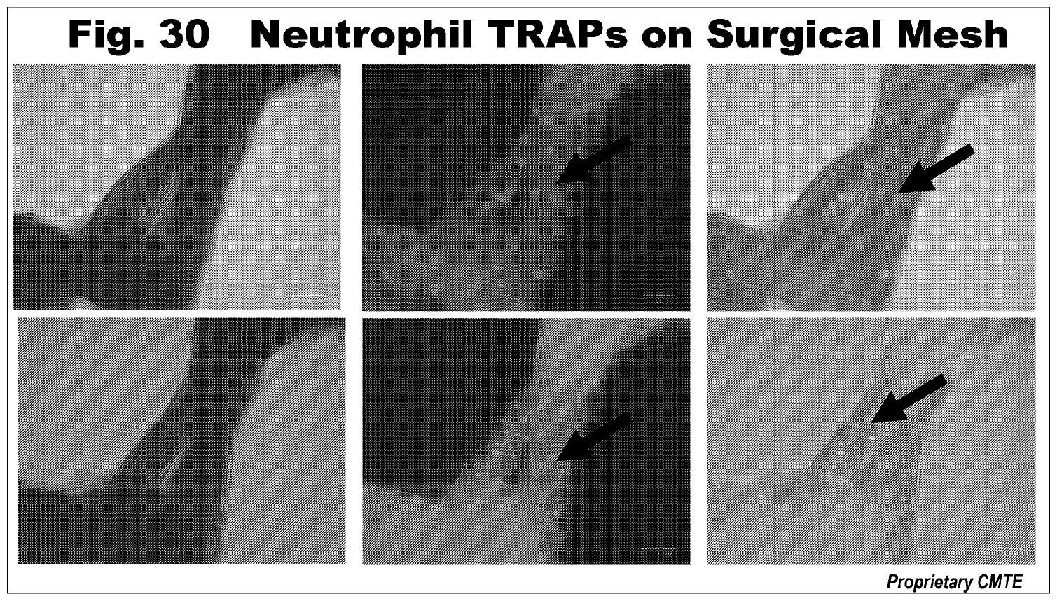

FIG. 30 the partner to FIG. 29, in which surgical mesh were placed in tissue culture media with the addition of neutrophils. Images were taken overtime to detect the presence of the TRAPS. The image demonstrates the interaction of neutrophils with the surgical mesh material, which parallels TRAPS seen in vivo with other devices, materials or fluids.

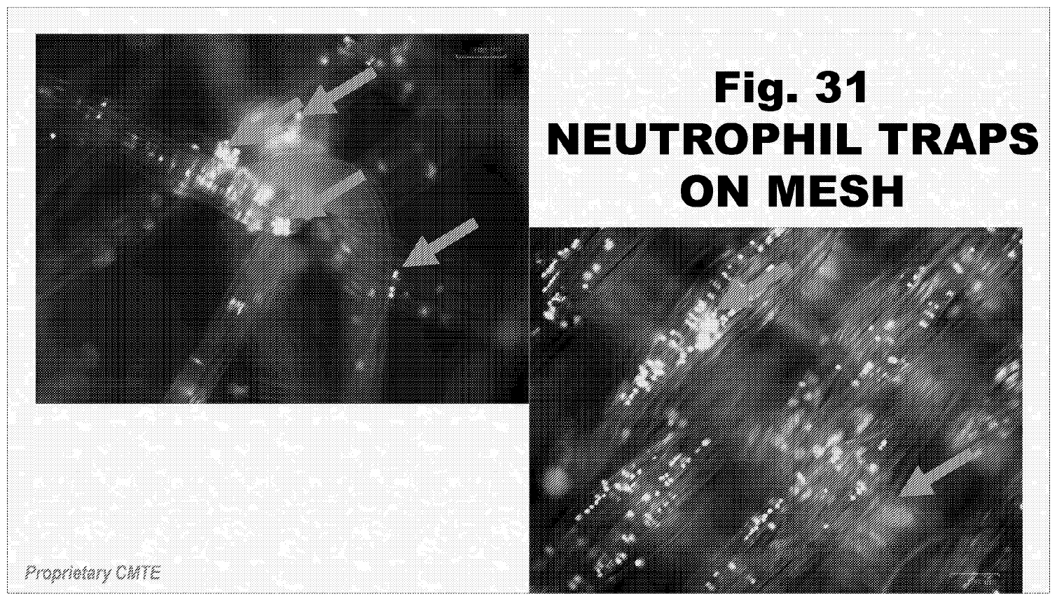

FIG. 31 is the partner to FIG. 30, in which different surgical meshes were placed in veteran tissue culture media with the addition of neutrophils. Images were taken over time to detect the presence of TRAPS. As can be seen here is the interaction of neutrophils with the surgical mesh material, which parallel TRAPS seen in previous slides using other devices, materials or fluids.

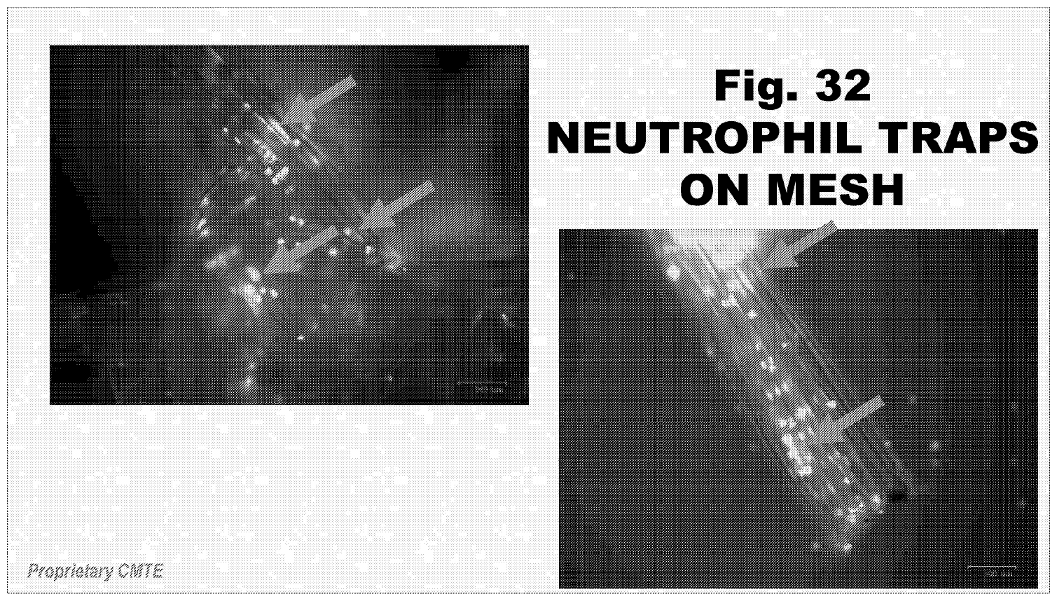

FIG. 32 shows mesh with TRAPS, indicated in green (light color in grayscale).

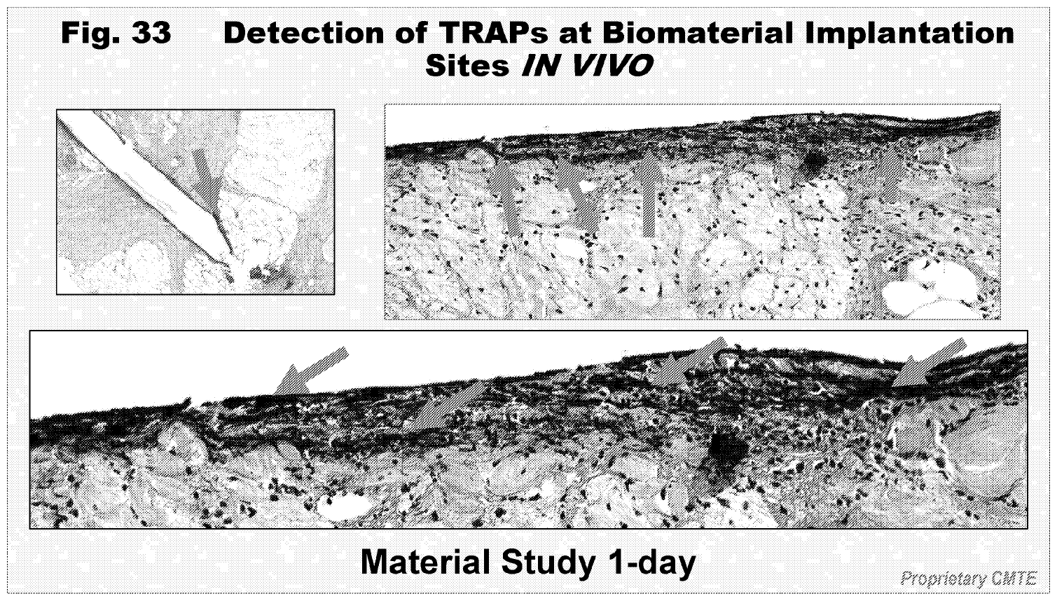

FIG. 33 In this case, pieces of raw biomaterial were implanted into the swine skin at various times. Biopsy was taken at day 1 and sections were made and stained with H&E. Presence of TRAPS (red arrows) at the interface between the test biomaterial (white open areas) and TRAPS. One can use TRAPs as screens too for biocompatibility of new materials for implantable medical devices.

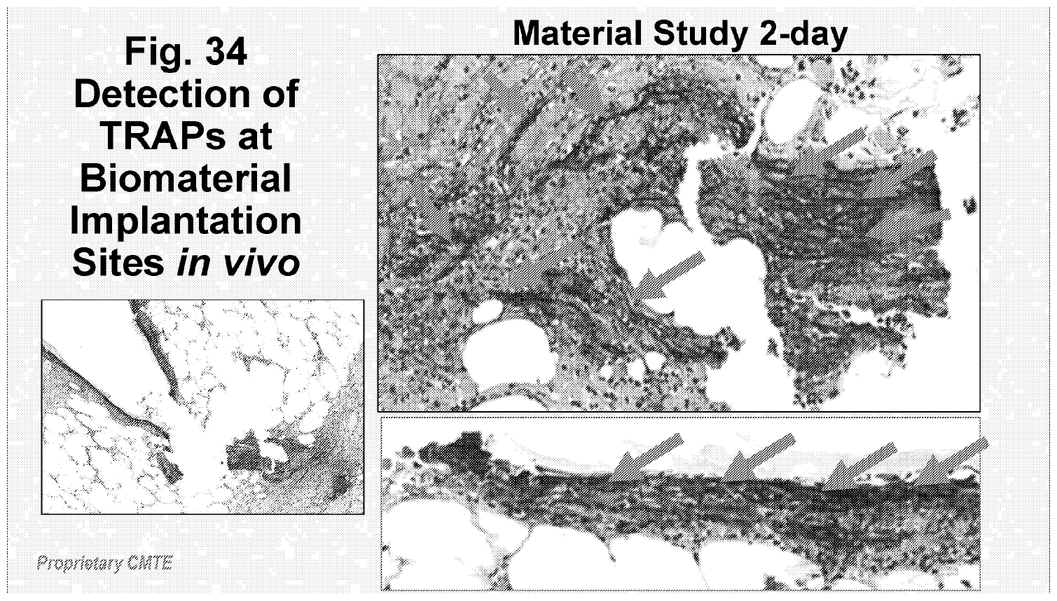

FIG. 34 Raw biomaterial were implanted into the swine skin pig and at various times a biopsy was taken at day 2, sections were made and stained with H&E. Tissue section demonstrate the presence of TRAPS (red arrows) at the interface between the test biomaterial (white open areas) and TRAPS. Can use TRAPs as screen too for biocompatibility of new materials for implantable medical devices.

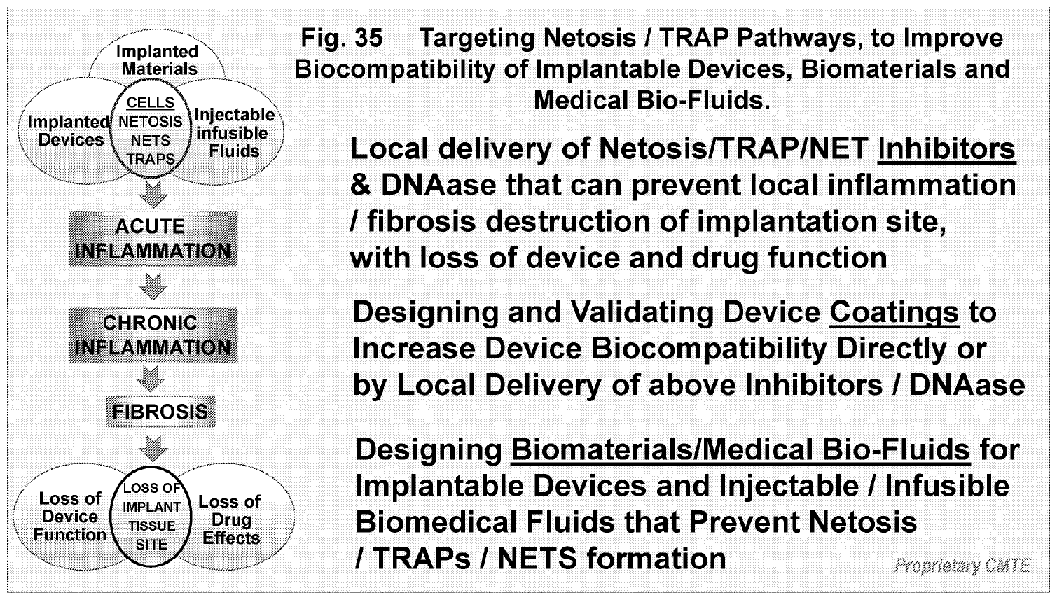

FIG. 35 describes how TRAPs and TRAP inhibitors and DNA degrading enzymes and inhibitors of things stuck to TRAPs like elastase MPO and other screening tools can be used for testing biocompatibility and preventing TRAP induced inflammation. TRAPs otherwise cause scarring that limits device material lifespan function and destroy normal health tissue that cannot be used in the future.

### “Air Pouch” Model

The air pouch model (of U.S. patent application Ser. No. 17/083,989) and included here has been used for the evaluation of tissue responses to tissue irritants and/or for the evaluation of tissue reaction inhibitors. The inventors adapted this model for the evaluation of tissue responses to infusion of insulin, excipients, factors, drugs and control solutions (e.g., saline). An example of air pouch model response to infusion of saline or insulin excipient is present in U.S. patent application Ser. No. 17/083,989. In embodiments, fluids other than air also can be used.

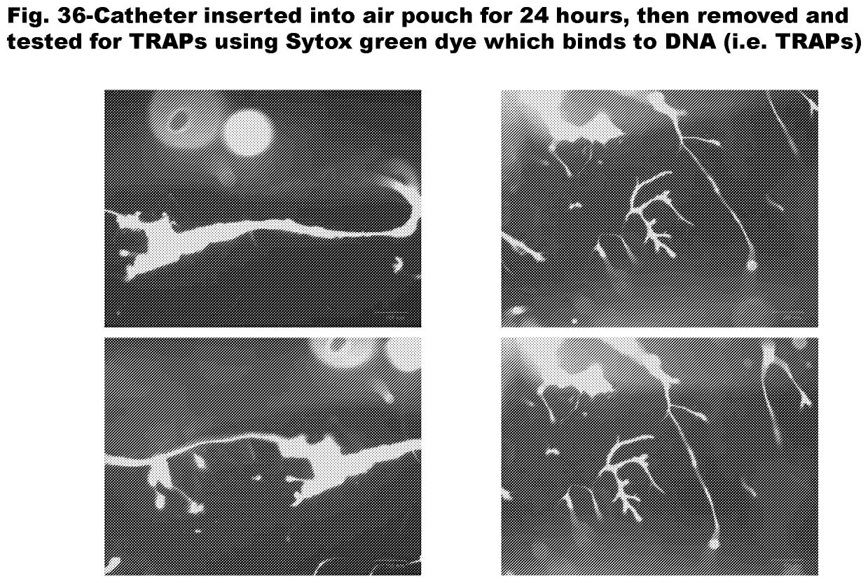

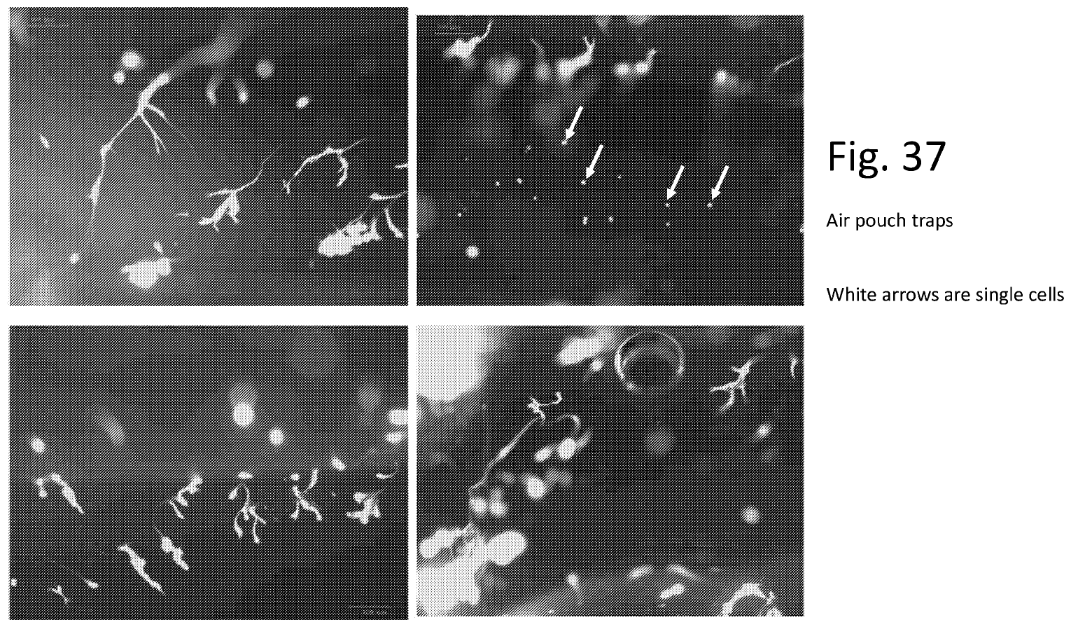

FIGS. 36-37 show air pouches (FIG. 36) for testing biomaterials of devices in vivo then remove them and put them under microscope to see TRAPs (FIG. 37).

Methods and uses of TRAPs and air pouch models can be used for testing of medical bio-fluids 2) medical devices and 3) biomaterials to determine their in vivo biocompatibility. The air pouch model and TRAPS to evaluate 1) anti-TRAP agents, which may suppress medical benefits of medical bio-fluids, 2) medical devices and 3) biomaterials TRAP inducing activity and destructive tissue reactions.

### Experimental Approach (Non-Limiting Example)

1) Skin air pouch induced in mouse skin by injection of air.
2) 24 hr. later catheter segments were placed in air pouch, and
3) 24 hr. later the catheter segments were removed from the air pouch.
4) Resulting segments suspended in tissue culture media for 30 minutes with SYTOX® Green, a fluorescent dye that can stain DNA, but does not permeate the plasma membrane, to demonstrate the presence of DNA TRAPs.
5) The resulting segments were viewed using an epi-fluorescent microscope.
6) Results were documented digitally.

The air pouch studies demonstrated that if the catheter is inserted into air pouch, it would trigger TRAP formation on the surfaces of the catheter. Thus, air pouch can be used to detect TRAPS formed in vivo in response to medical devices, medical bio-fluids and medical bio-material, using DNA dyes to stain nucleated cell-derived TRAPS on the surface of the segments that had been placed in the air pouch (see FIGS. 36 and 37). The air pouch model can also be used to test anti-TRAP agents for uses in vivo. Using air pouch and net trap dyes, the inventors can evaluate ability of medical devices, medical bio-fluids and medical bio-material for their ability to trigger TRAPs (companies interested in this), as well as testing coatings and devices, basic medical bio-material and medical bio-fluids. Also, the inventors can treat the animals systemically or locally with medical bio-fluids (drugs, DNases, inhibitors, that can prevent, block or remove nets/TRAPs in vivo to predict their performance in patients and animals. This enables us to determine who needs to be protected from damage of nets/TRAPs in vitro and in vivo.

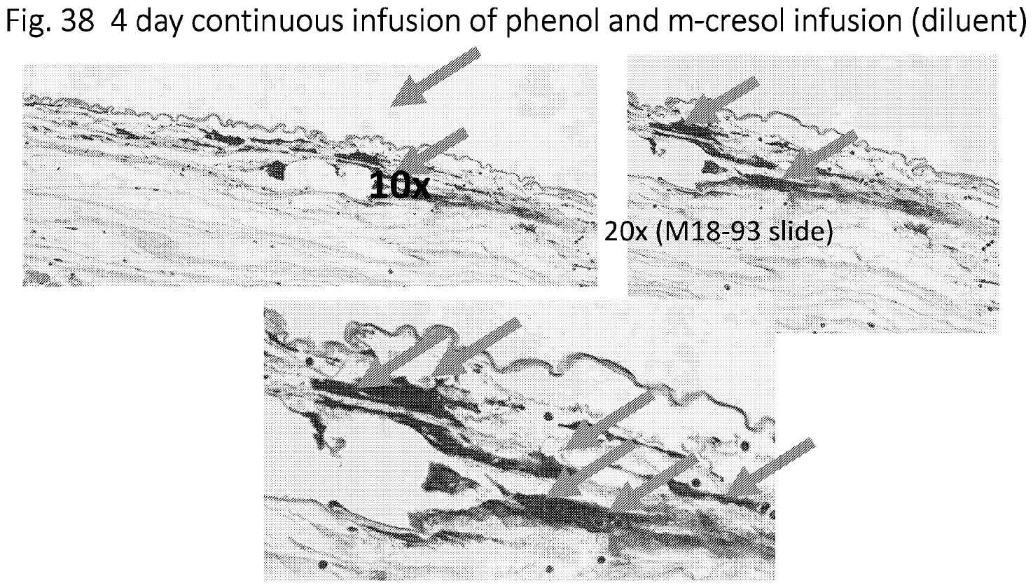

FIG. 38 shows photos of 4-day continuous infusion of phenol and m-cresol infusion (i.e., phenol and m-cresol are used as insulin diluent in commercial insulin formulations) in pig skin. This figure demonstrates the formation of TRAPs at phenolic infusion sites in pigs. TRAPs are indicated by red (gray) arrows.

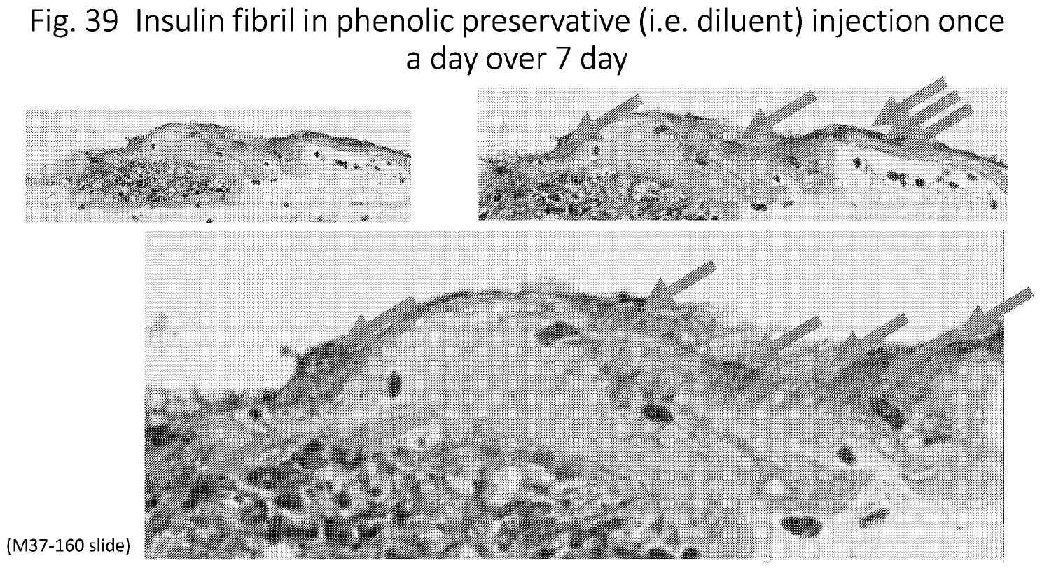

FIG. 39 illustrates insulin derived fibrils in phenolic preservative (i.e. diluent) injection once a day over 7 days. This demonstrates that insulin fibrils (non-functional insulin polymers) trigger TRAPs in pig skin at 7 days post infusion. Inventors also have shown that insulin fibrils without diluent can trigger TRAP formations.

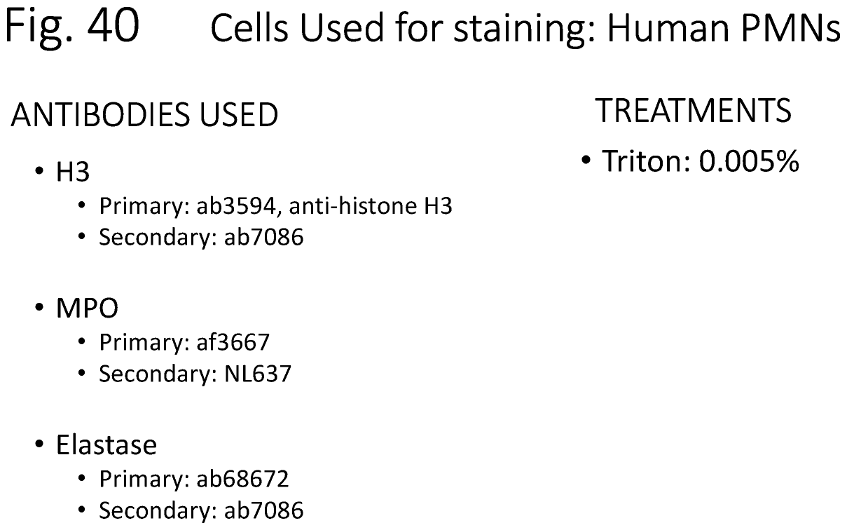

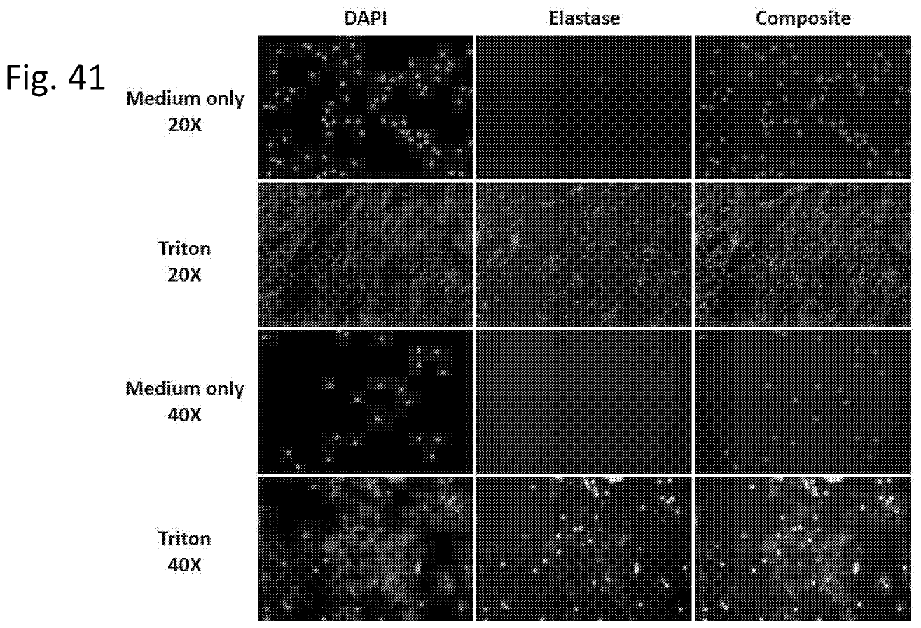

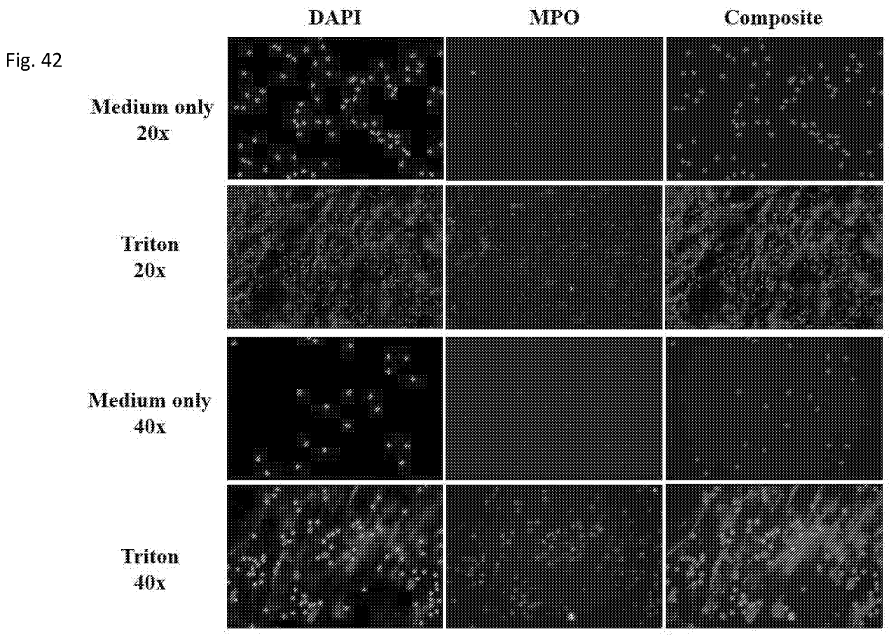

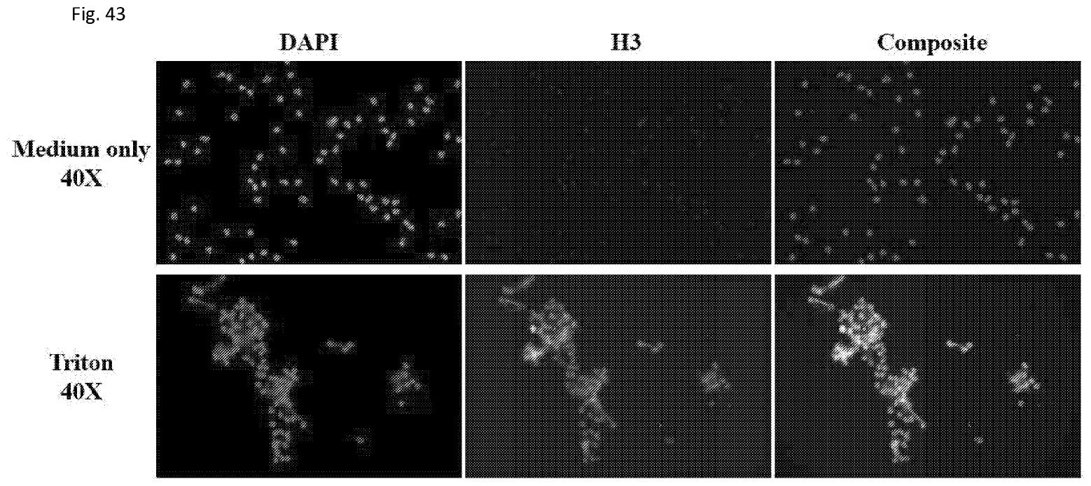

FIG. 40-43 show the ability of various antibodies that can detect cell factors that can bind to DNA (including DNA in TRAPs during netosis and after TRAP release for cells). More specifically, FIG. 41 shows anti-elastase antibodies co-localize with DNA (DAPI) in release for neutrophils. FIG. 42 shows anti-MPO antibodies co-localize with DNA (DAPI) in release for neutrophils. FIG. 43 shows anti-histone antibodies colocalize with DNA (DAPI) in release for neutrophils.

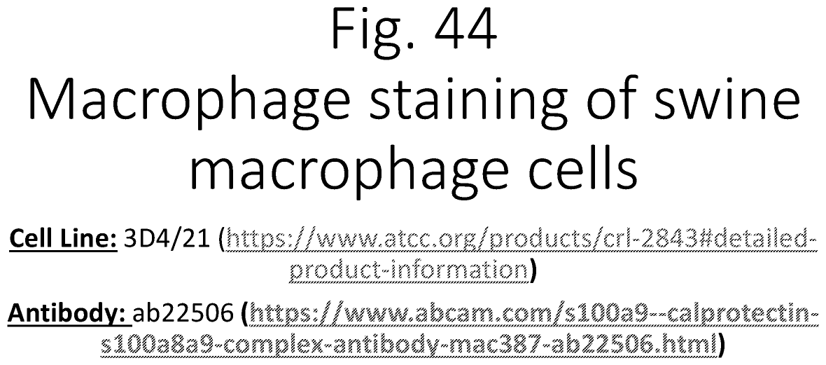

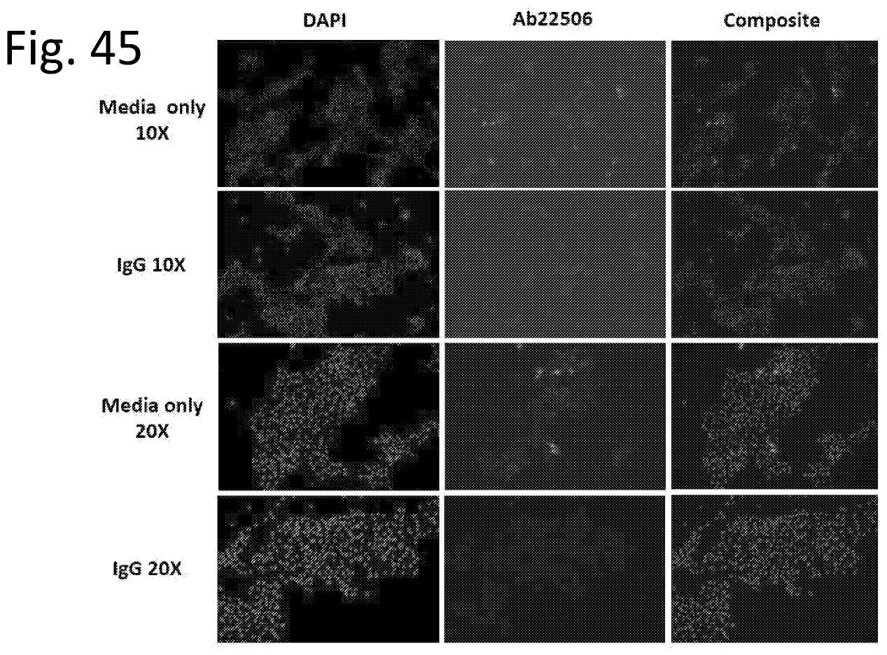

FIGS. 44-45 relate to macrophage staining of swine macrophage cells when a particular antibody is used. As is shown in FIG. 45, the anti-body reacts with macrophages, compared to normal IG controls.

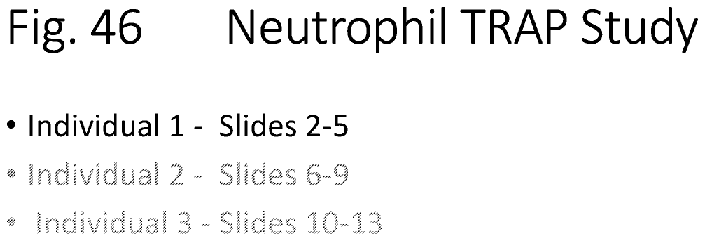

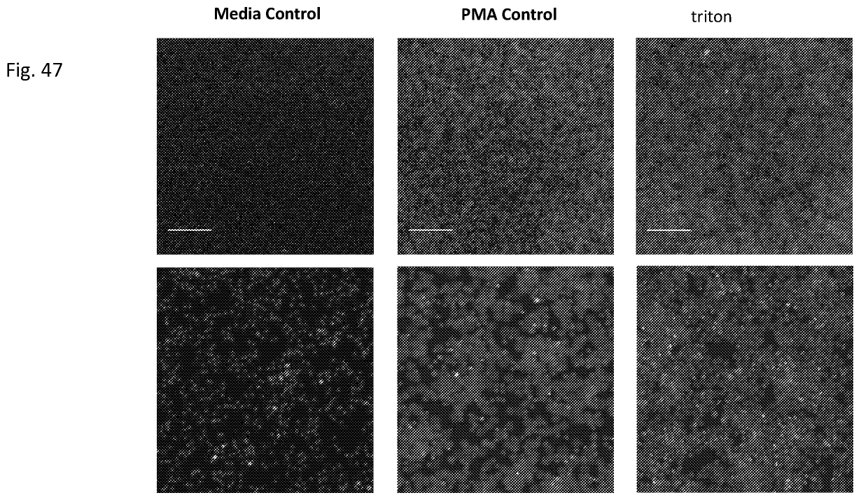

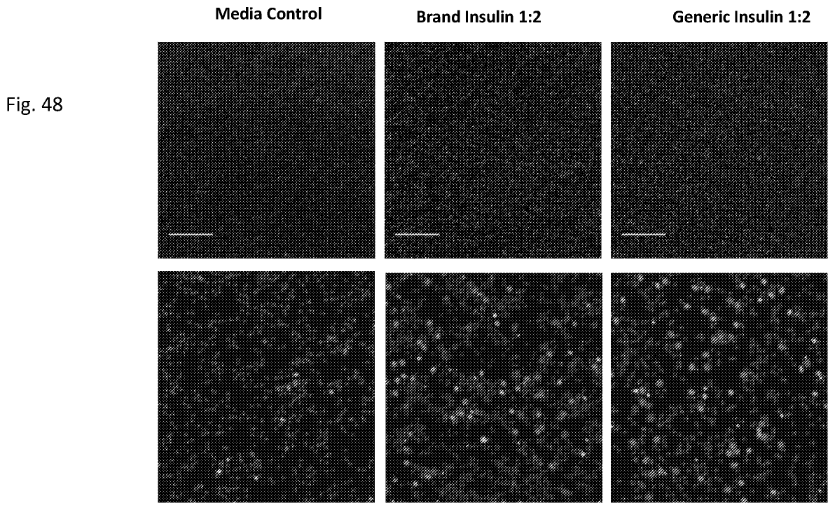

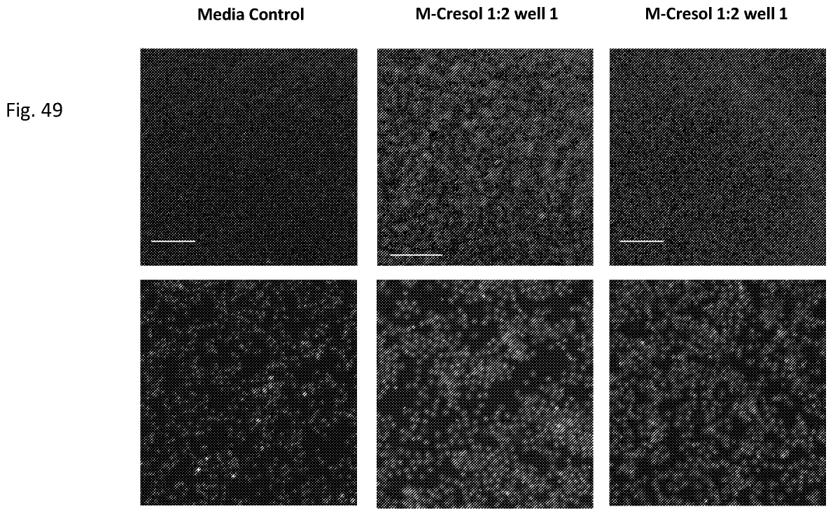

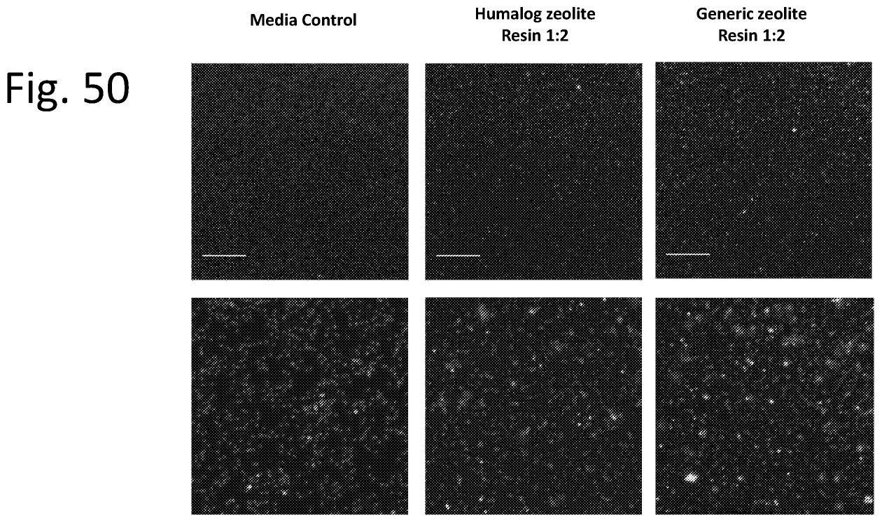

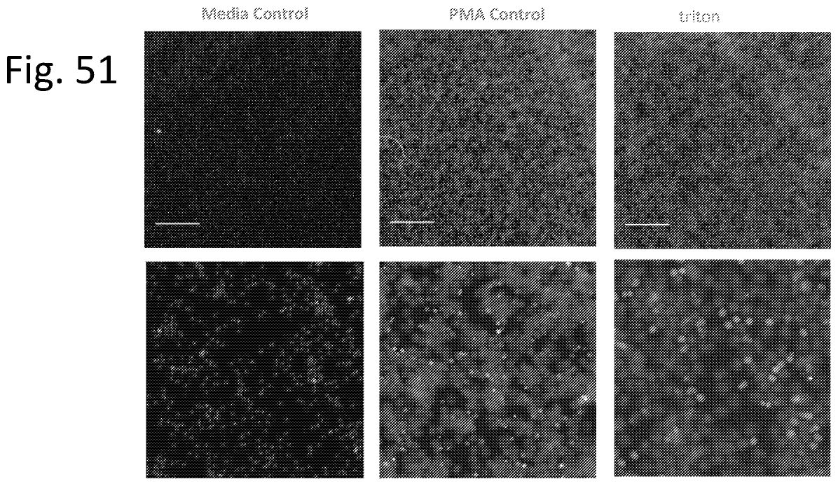

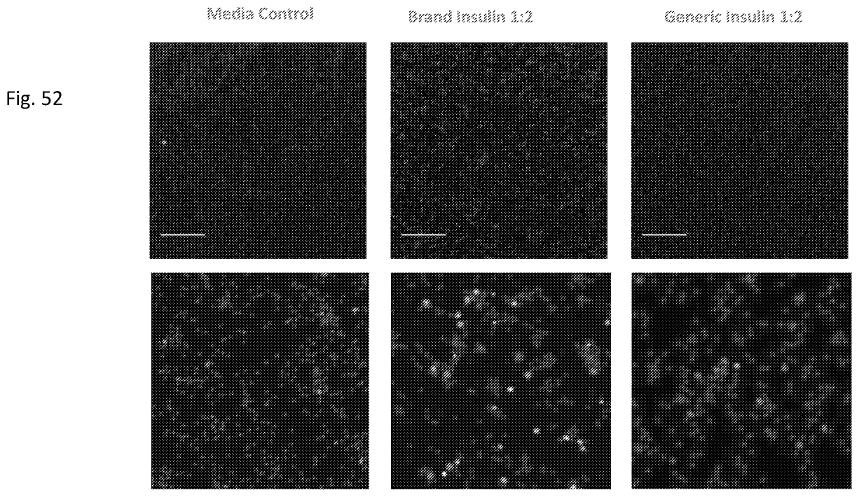

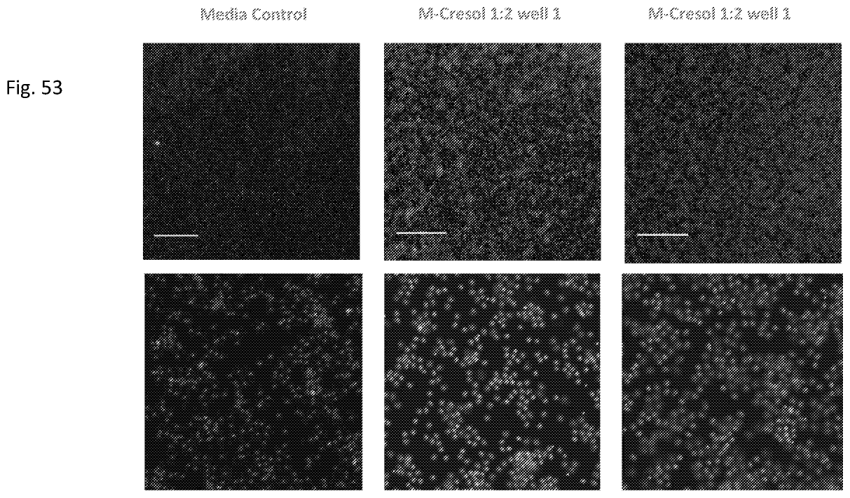

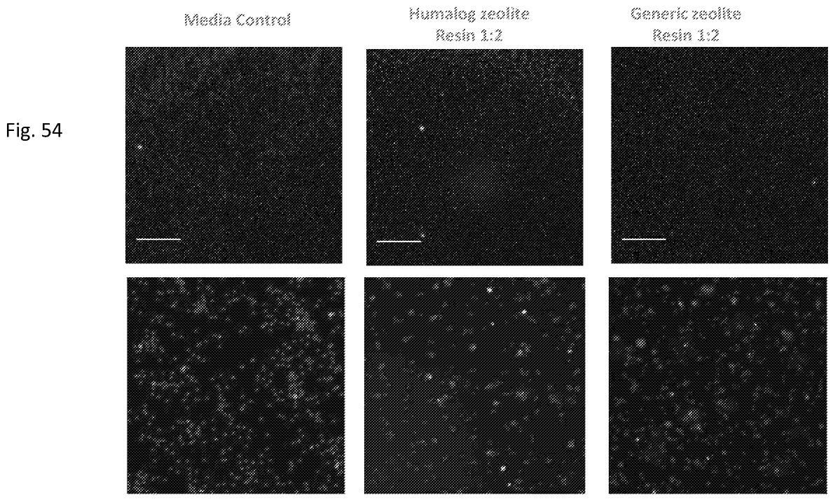

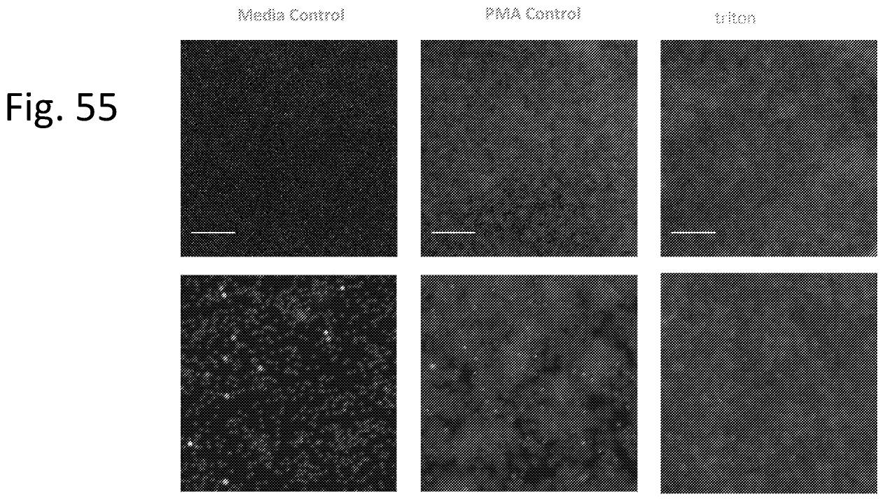

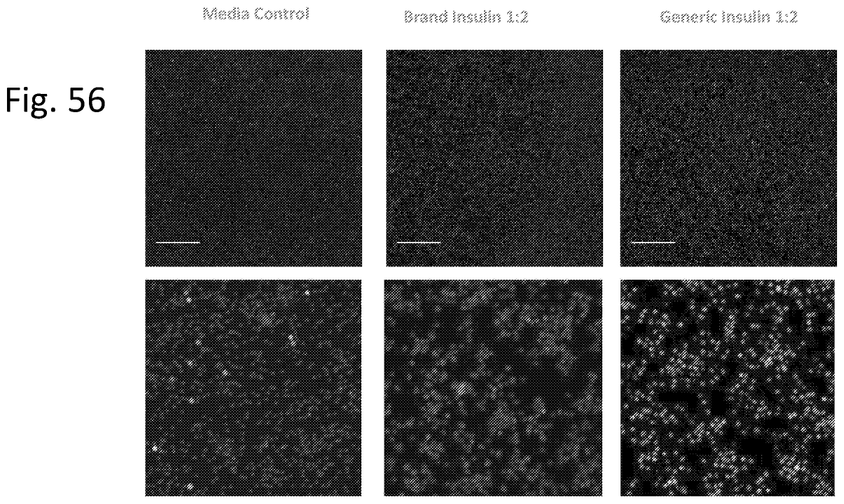

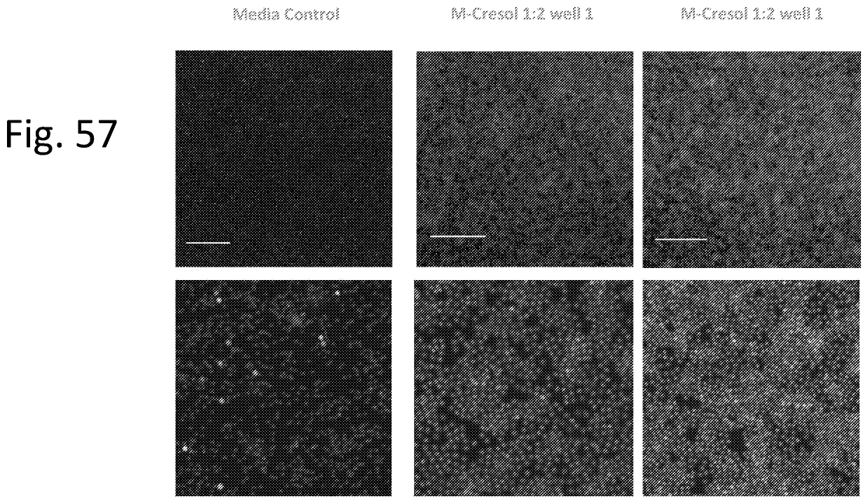

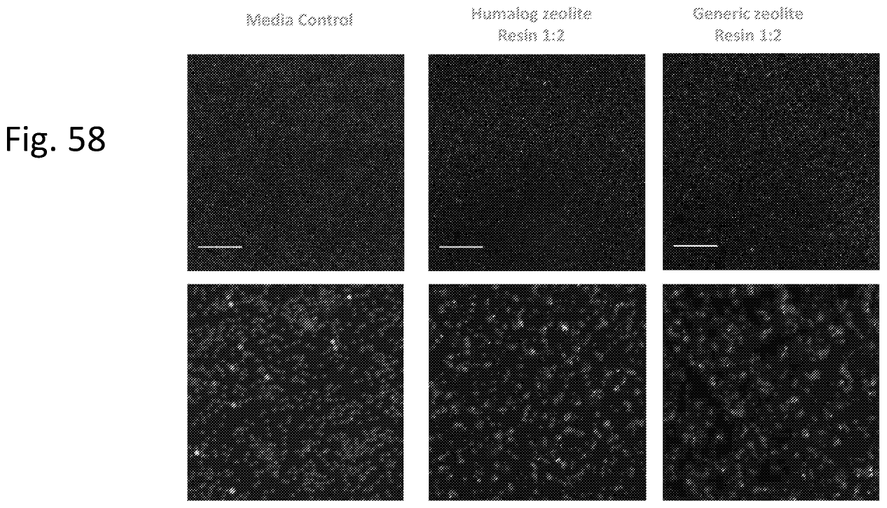

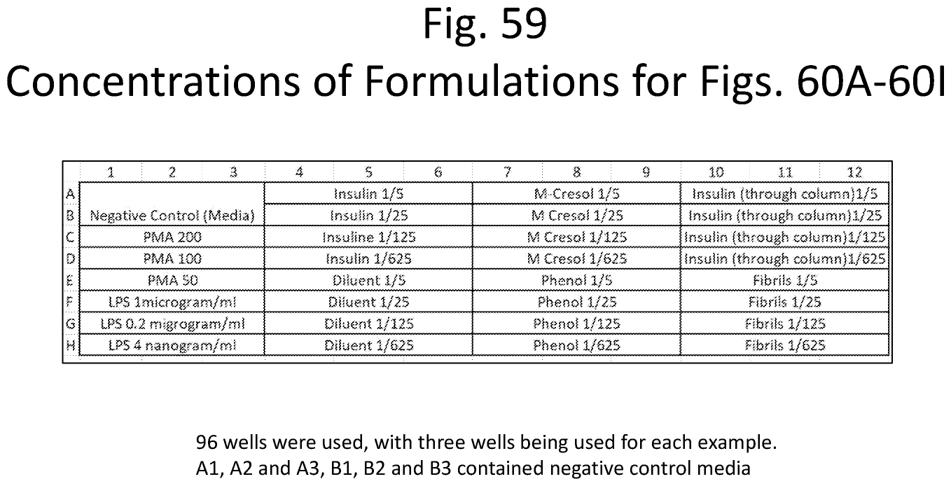

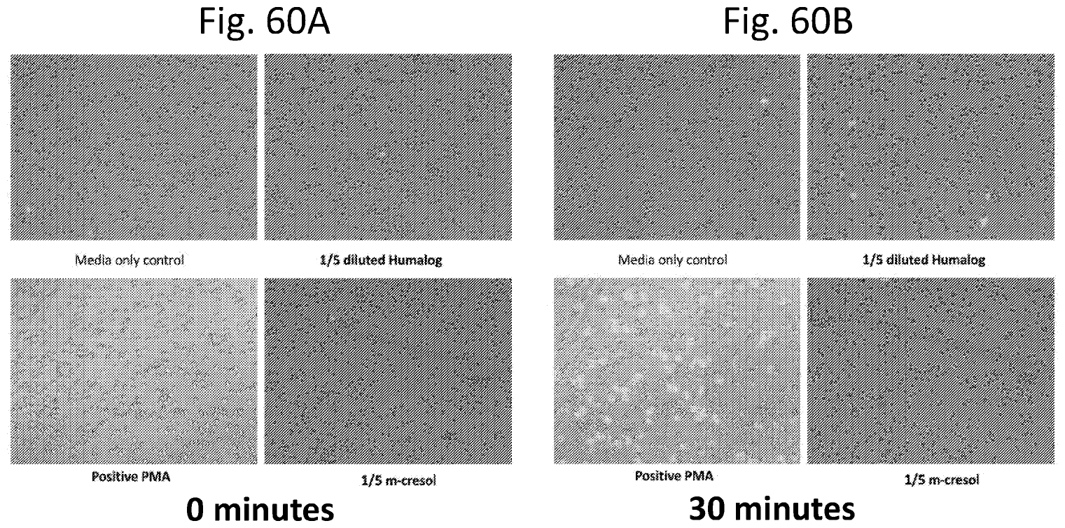

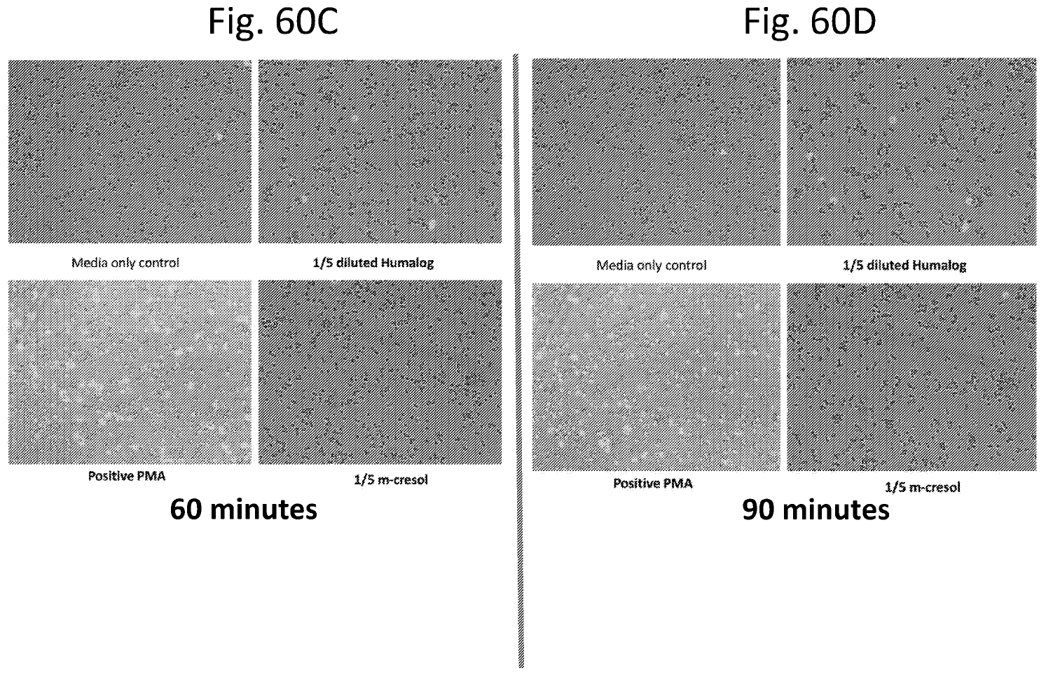

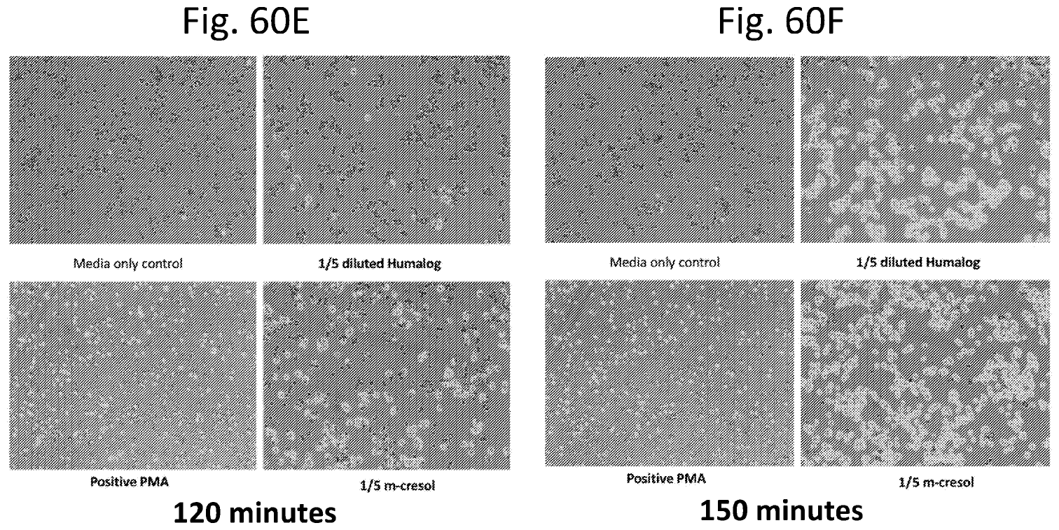

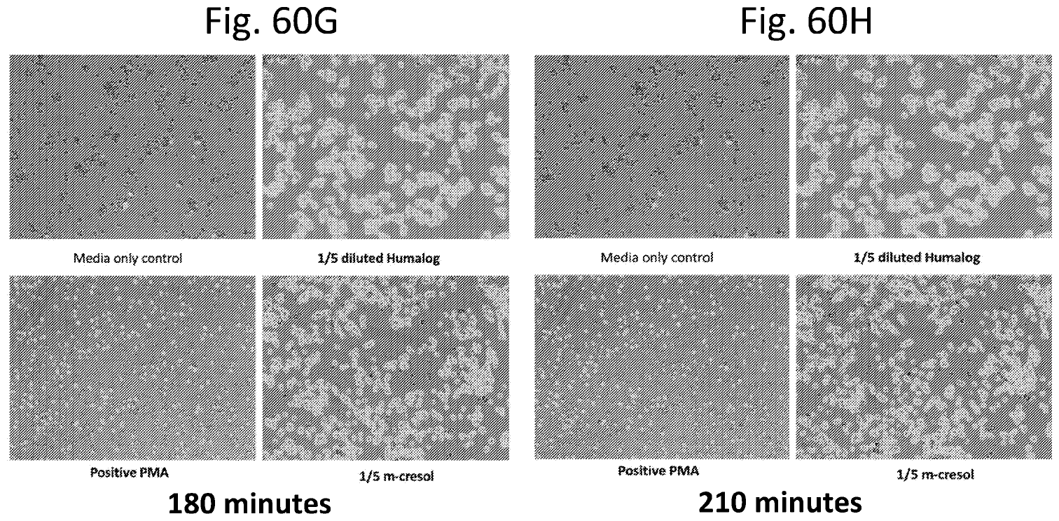

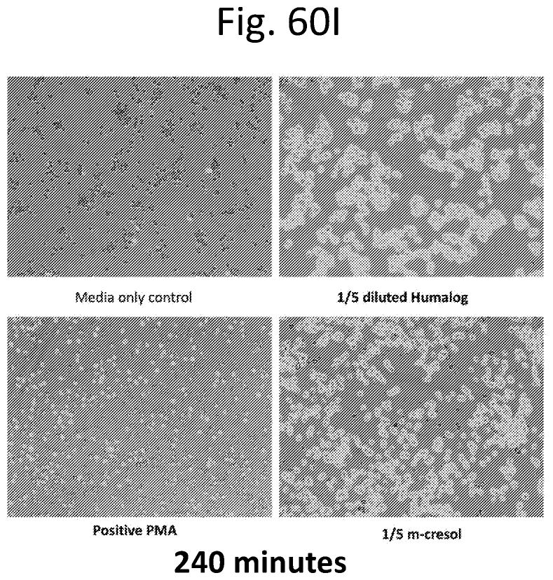

FIGS. 46-58 show the formation of TRAPs as a result of PMNs from 3 different normal donors, over a time period of 6 hours for 3 PMNs from 3 normal individuals. As can be seen in the photos, insulin formulations lead to the formation of TRAPs, probably due to the presence of commercial insulins, phenol and/or m-cresol. Importantly, these studies also demonstrate that if the phenolics (PP) are removed from the commercial insulin preparations, the resulting PP depleted commercial Insulins do not induced traps FIG. 50, 54, 58 (for the 3 normal donors) FIG. 59 shows the concentrations of the liquid formulations studied and illustrated in FIGS. 60A-60I, show time and concentration studies of the ability of commercial insulin, m-cresol, PMA (positive control). These studies demonstrated that 1/5 dilution of commercial insulin and m-cresol trigger traps within the 60-120 minutes after exposures, that PMA triggers within 60 minutes post exposures FIG. 59, and FIGS. 60A-60I). PMA=phorbol myristate acetate; Insulin is Humalog (Lilly); LPS is a bacterial endotoxin (known inducers of TRAPs.

Specifically, FIG. 60A shows an absence of TRAPs on all slides at 0 minutes. FIG. 60B shows the presence of TRAPs at 30 minutes in PMA, which is a known inducer of TRAPs. FIG. 60C shows significant TRAPs in PMA at 60 minutes and several TRAPs in the insulin. FIG. 60D shows a slight increase in TRAPs in insulin at 90 minutes. FIGS. 60E and 60F show a significant increase in the presence of TRAPs in Humalog insulin and m-cresol from 120 minutes to 150 minutes. FIGS. 60G and 60H show TRAPs at 180 and 210 minutes, and FIG. 601 shows TRAPs at 240 minutes. The number and size of TRAPs remained relatively stable in insulin and m-cresol from 180 minutes to 240 minutes.

FIGS. 60A-60I show the appearance of TRAPs over time in insulin and insulin preservatives as compared to controls. Green color (light color in grayscale) indicates the existence of TRAPs. In each slide set, the upper left slide is the control with media only. The upper right slide is Humalog insulin in media at 1/5 dilution (0.6 mg m-cresol/ml, with 0.6 mg/ml of insulin), PMA concentrations are in ng/ml and the lower right slide is m-cresol at 1/5 dilution (0.6 mg/ml of m-cresol). In this study, the media was RPMI 1640 (phenol free).

FIG. 60A shows an absence of TRAPs on all slides at 0 minutes. FIG. 60B shows the presence of TRAPs at 30 minutes in PMA, which is a known inducer of TRAPs. FIG. 60C shows significant TRAPs in PMA at 60 minutes and several TRAPs in the insulin. FIG. 60D shows a slight increase in TRAPs in insulin at 90 minutes. FIGS. 60E and 60F show a significant increase in the presence of TRAPs in Humalog insulin and m-cresol from 120 minutes to 150 minutes. FIGS. 60G and 60H show TRAPs at 180 and 210 minutes, and FIG. 601 shows TRAPs at 240 minutes. The number and size of TRAPs remained relatively stable in insulin and m-cresol from 180 minutes to 240 minutes.

Additional studies in this set include demonstration that removal of the PP form the commercial insulin removed all TRAP inducing activity in commercial insulin (Humalog) inducing activity of the commercial insulin (Humalog). Also, these studies demonstrated that fibrils (polymers of insulin (i.e. insulin fibrils) that lacked an PP, could also triggered trap formation form all 3 populations of donor PMNs.

Summaries of these studies indicated that Negative controls are PMN+media (negative controls), few TRAPs form. The additional studies done at the same time with these 3 population of PMNs list in (see FIG. 59) demonstrated that commercial insulin at 1/5, commercial, insulin diluent from Lilly (used to dilute commercial insulin before injections) at a dilution of 1/5, phenol at a dilution of 1/5, and insulin fibrils at a 1/5 dilution without any preservatives (PP) all triggered TRAP formation in the same 4 hr timeline, when compared to negative control PMS (i.e. no treatment). Additionally, removal of PP from the commercial insulins block trap formation at any timepoint or concentration of the commercial insulin. Positive control PMA and LPS (i.e. known TRAP inducers) trigger TRAP formation in all 3 populations (3 donors) of PMNs.

PMA is a chemical positive control for inducing TRAPs in PMNs at 3 concentrations: PMA triggered TRAPs as predicted. LPS is bacterial cell walls (also known as endotoxin), another positive control for TRAPs: as predicted, the LPS triggered TRAPs in PMNs. Insulin is commercial insulin which contains phenolics (Humalog from Lilly) at 3 concentrations 1/5 dilution strong TRAP formation, 1/25 dilution weak TRAP formation, 1/125 dilution no TRAP formation when compared to negative controls (cells only).

Diluent used to dilute insulin for pediatric applications: contains both [phenol and m-cresol) showed the same TRAP pattern as the commercial insulin from Lilly: 1/5 strong higher dilution weak to no TRAP formation. M-cresol (about 3 mg/ml) is phenolic present on Humalog (no phenol present just m-cresol) and diluent for Lilly has both phenol+m-cresol (total of about 3 mg/ml): 1/5 strong higher dilution weak to no TRAP formation. Phenol (about 3 mg/ml) is phenolic present on diluent (Lilly as well as other commercial insulin Humalog (no phenol present just m-cresol) and diluent for Lilly has both phenol+m-cresol (total of about 3 mg/ml): 1/5 dilution strong traps, higher dilution weak to no TRAP formation.

Insulin thru column is Humalog run thru zeolite resin column to remove m-cresol but does not remove insulin (to show insulin does not trigger TRAPs) and we found that removal of the m-cresol removed all the TRAP formations induced by the Humalog with m-cresols. This supports the idea that m-cresol and or phenol trigger the TRAPs and not the insulin in the commercial insulins. Fibrils refers to insulin derived fibrils (insulin polymers) (these insulin fibrils are not functional as far as reducing blood glucose). But they are particulate and can be ingested by white blood cells and can trigger TRAP formation (I can check over what range) 1/5 dilution are equivalate to the same amount of Humalog insulin as we used in the insulin studies cited in this plate and the other studies.

As indicated above in the section of this document describing anti-TRAP agents, non-limiting examples of TRAP inhibitors are listed on FIGS. 68-71.

In addition to the examples listed on FIGS. 68-71, in embodiments, the following inducers can be used when TRAPS are to be induced:
A23187 (calcimycin)—calcium ionophore
Ionomycin—a calcium ionophore
Phorbol 12—myristate 13 acetate (PMA)—phorbal ester

In addition to the examples indicated in other parts of this document, the following TRAP inhibitors can be used:
4-aminobenzoic acid hydrazide
Alexidine—alkyl bis(biguanide) antiseptic, which is used in mouthwash
Aspirin
AZD-7986—also known as cathepsin C
Azithromycin—a macrolide antibiotic
BB-Cl-Amidine
BMS-B5—an inhibitor of PAD4
Capsaicin—a terpene alkaloid
Transient receptor potential vanilloid type 1 (TRPV1)
Chloroquine—an aminoquiloline
Cl-amidine—an irreversible inhibitor of PADs
CP-673-451
Cyclosporin A—an immunosuppressant drug that binds cyclophilin D
Dihydrocapsaicin—a terpene alkaloid
Diphenyleneiodinonium—(DPI)—an inhibitor of NADPH oxidase
Doxorubicin—an anthracycline antitumor antibiotic
Epirubicin—a steoeoisomer of doxorubicin
6-Gingerol, 8-Gingerol and 10-Gingerol—derivatives of ginger
GSK-484—a reversible inhibitor of PAD4
Raf-1 (GW 5074)—a proto-oncogene serine-threonine protein kinase
GW311616A—neutrophil elastase inhibitor
Hydroxychloroquine (sulfate)
Idarubicin—a 4 demethoxy analogue of daunorubicin
IM-93—(Item No. 28794 from Cayman Chemical) a dual inhibitor of netosis and ferroptosis
Jasplakinolide—macrocyclic peptide first isolated from a marine sponge
Metformin—a biguanide
Nigericin—an antibiotic
Nonivamide—a TRPVT agonist
PF-1355—a MPO inhibitor
Prostaglandin E2 (PGE2)
R406—ATP competitive inhibitor of spleen tyrosine kinase
Spleen tyrosine kinase (Syk)
R-848—immune response modifier
Sivelestat—inhibitor of neutrophil elastase
SP6000125—pan-inhibitor of JNK
TAK-242—antagonist of TLR4
U-0126—MEK inhibitor

These inducers and inhibitors are available from various sources, including Cayman Chemical and Selleck. Cayman Chemical also provides a netosis screening set, marked as Cayman 35019, as well as anti-inflammatory screening library, and a cell death screening library. Using these libraries in vitro and in vivo help to determine the specific factors and pathways that induce and stop trap formation, identify the trap composition as well as help in identifying trap biomarker and treatments.

Substances that inhibit the formation of TRAPs include intracellular inhibitors and extracellular inhibitors. An intracellular inhibitor inhibits the formation of TRAPs. An extracellular inhibitor prevents toxic elements of an existing TRAP from causing tissue damage, including inflammation and fibrosis. Some substances function as both intracellular inhibitors and extracellular inhibitors. Hydroxychloroquine is known to inhibit ROS, Il-8, PAD4 and Rac2. MTX is known to inhibit ROS and leukotriene B4, and induce adenosine production. Prednisolone is known to inhibit ROS and inflammatory cytokines, PAD4, Elastase and MPO inhibitors include IPF-1355, AZD9668 and BMS-P5. Rituximab and Belimumab are known for B cell depletion, reduction in B cell survival, and inhibiting ANA production. Tocilizumab is known to increase endothelial function and inhibit ROS. Cl-amidine inhibits PAD4. DPI inhibits NOX and ROS. Other inhibitors include Rapamycin, Lapatimib, and Bosutimib. Protease inhibitors like Sivelestat and GW311616A, target elastase, and AZD-7986 inhibit cathepsin C in cell and in extracellular DNA traps. To target oxygen metabolites, anti-oxygen metabolites such as Diphenyleneiodinonium can be used.

Non-limiting examples of substances that neutralize TRAPs include DNase and antibodies.

Embodiments disclosed herein reduce TRAP formation and netosis caused by medical biofluids, such as preservatives. Non-limiting examples of medical preservatives are shown below. Benzene-containing compounds such as phenol and m-cresol are used as preservatives. Several prevalent medical preservatives are antimicrobial preservatives such as phenol and benzyl alcohol, parabens, benzalkonium chloride (BAK) and polyquaterium-1 PQ-1. Known preservatives include ophthalmic preservatives. Four categories of ophthalmic preservatives are detergents, oxidants, chelating agents, and metabolic inhibitors (pentavalent antimonials [SbV], quaternary ammoniums, and organomercurials).

Preservative concentrations recommended for parental preparation

| Sr. No. | Name | Recommended Concentration |
|---|---|---|
| 1. | Benzyl Alcohol | 0.5 to 10% |
| 2. | Benzalkonium Chloride | 0.01% |
| 3. | Butyl Paraben | 0.015% |
| 4. | Chlorobutanol | 0.25 to 0.5% |
| 5. | Meta Cresol | 01 to 0.25% |
| 6. | Chlorocresol | 0.1 to 0.18% |
| 7. | Methyl Paraben | 0.01 to 0.5% |
| 8. | Phenyl Ethyl Alcohol | 0.25 to 0.002% |
| 9. | Propyl Paraben | 0.005 to 0.002% |
| 10. | Phenol | 0.065 to 0.02% |

Preservative Concentration for Liquid Oral Preparation

| Sr. No. | Name | Recommended Concentration |
|---|---|---|
| 1. | Benzonic Acid | 0.1 to 0.2% |
| 2. | Sorbic Acid | 0.1 to 0.2% |
| 3. | Methyl Paraben | 0.25% |
| 4. | Propyl Paraben | 0.5 to 0.25% |
| 5. | Sodium Benzonate | 0.1 to 0.2% |
| 6. | Bronidol | 0.001 to 0.05% |
| 7. | Propylene Glycol | 0.25% |

### Specific Solutions—Preventing Inflammation by Preventing or Inhibiting TRAP Formation

The Examples provided above address tissue infection and injury resulting from insulin injection and continuously infused insulin can cause inflammation, which leads to the loss of viable tissue for continuous subcutaneous insulin infusion and fibrosis.

Diabetes and other Diseases. While the following paragraph address diabetes and insulin formulations, they also apply to other medical bio-fluids, implantable devices and non-implantable devices.

Artificial pancreas system requirements include the need to maintain precise and accurate in vivo delivery of very minute and continuously variable amounts of insulin in response to changing blood glucose. Additionally, the physical absorption and BG response to infused insulin should remain constant, permitting stable AP algorithm performance. Based upon our recent work, we understand that insulin infusion triggers tissue injury and local inflammatory responses at insulin infusion sites, which ultimately results in limited infusion site longevity, premature infusion failure and PK absorption variability. The inventors also understand the IFP trigger tissue injury and local inflammatory reactions (inflammation and fibrosis) both during infusion and afterwards (i.e. after cannula withdrawal), that ultimately limit infusion site longevity, infusion failure and PK absorption.

Problem 1. Insulin, insulin additives and their products are cell and tissue toxic, as well as immunomodulatory, and induce inflammation, loss of blood and lymphatic vessel and scarring at sites of insulin injection and infusion.

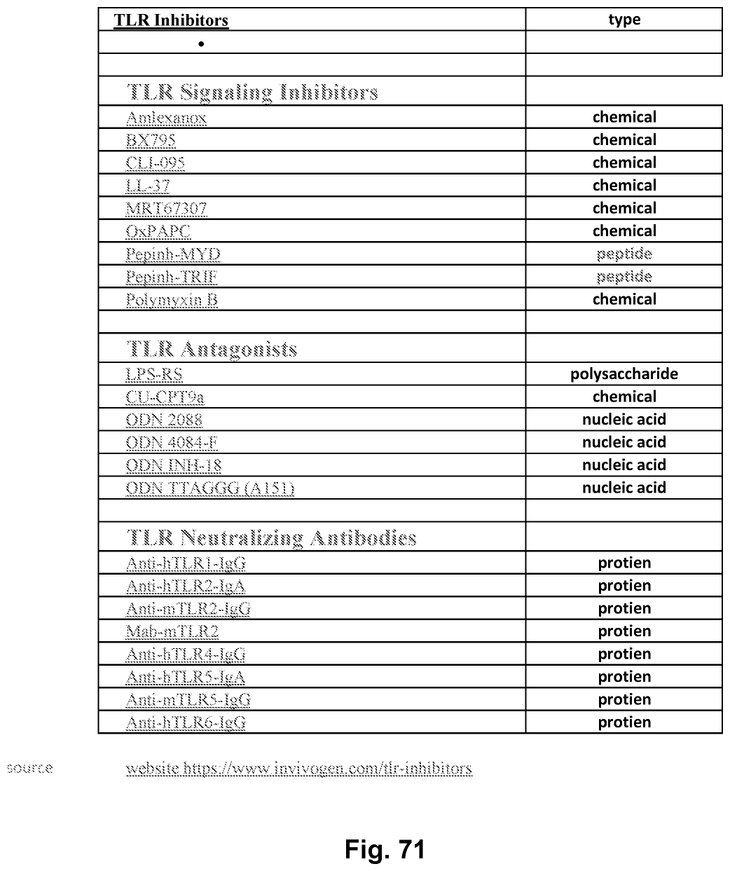

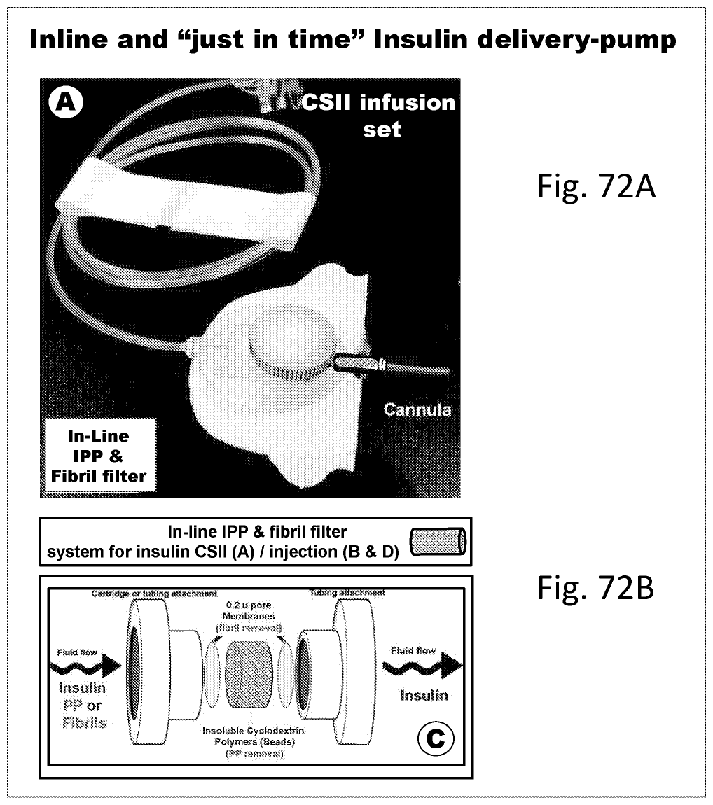

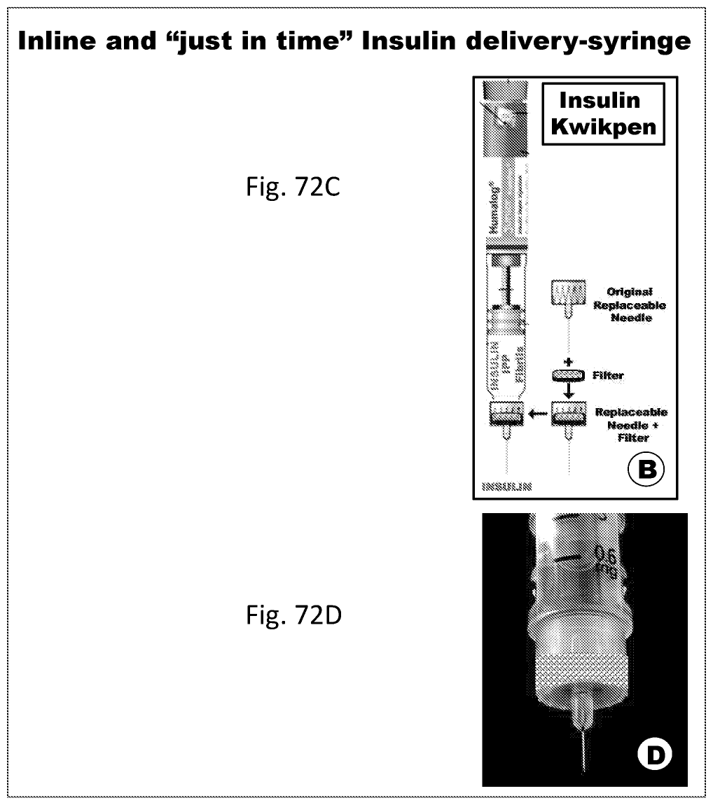

Solution for Problem 1. Employ “In-line” device for inhibiting the formation of TRAPs. This can be accomplished by removing and adding substances using the in-line device. FIGS. 72A-72B show an exemplary insulin pump with an in-line filtration device. The filter can be configured to remove materials from a liquid, or can slowly release an agent into the liquid.

Problem 2. Insulin, insulin additives and their products are cell and tissue toxic, as well as immunomodulatory, and thereby can increase and or decrease local tissue reactions at sites of insulin injections and infusion. This increase in site tissue reactions could lead to increased inflammation, and scarring which compromises short and long term insulin therapy for diabetes.

Solution for Problem 2. Employ collar-like barriers with added anti-TRAP agents in order to alleviate CSII associated infection(s). The inventors have developed a (tacky) silicone-based collar that contains an added antimicrobial agent and have demonstrated that this device attribute extends the functional lifespan of commercial glucose sensors in vivo. These same silicone collars, or collars made of other materials, can be used with current insulin infusion sets to extend tissue integrity at sites of insulin injections and infusion.

Problem 3. Not only add anti-netosis and anti-TRAP agents can add agents that suppress tissue reactions including anti-inflammatory, and anti-fibrosis agents as well as agents that promote healing such as agents that induce ingrowth of blood vessel and lymphatic inducing agents such as VEGF family of growth factors.

Problem 4. Because CSII requires insertion of the insulin cannula across the skin into the subcutaneous tissue layer, the insertion site remains an open wound for the period of infusion that exposes the underlying tissue to the risk of infiltrating pathogens and subsequent infection and the associated inflammation, scarring and loss of tissue integrity.

Solution A for Problem 4. Employ collar-like barriers with added-anti-TRAP agents in order to alleviate CSII-associated infection(s) and resulting inflammation that can compromise both short-term and long-term CSII tissue site integrity. The inventors have developed a (tacky) silicone-based collar, into which anti-TRAP/anti-TRAP agents can be incorporated, in order to extend the functional lifespan of commercial glucose sensors in vivo. The inventors believe these same silicone collars can be used with current insulin infusion sets to decrease infusion site infections, inflammation and tissue scarring at sites of device implantation.

Problem 5. Extended CSII causes increased adhesive damage to skin epithelium, thereby increasing the risk of infections, inflammation and scarring, all of which compromises short and long term insulin therapy for diabetes.

Solution for Problem 5. Employ “extended” collar-like barriers containing anti-TRAP agents.

Problem 6. CSII Cannula's induced tissue reactions and associated infections.

Solution A for Problem 6A. Employ a local anti-TRAP agent coated cannula to help minimize infections and inflammation and promote new blood vessel formation at sites of CSII and sites of other types of liquid infusions.

Solution B for Problem 6B. Develop pump based anti-TRAP agent delivery (single or dual lumen cannulas) to decrease infection, inflammation and fibrosis and induce new blood vessels at CSII infusion sites. An alternative of “coating” based drug delivery is to utilize the insulin pump system as part of an integrated insulin+drug delivery system. This could be done using a single or dual lumen system that could deliver insulin and drugs such as an anti-TRAP agent.

Problem 7. CSII induced tissue reactions and infection risk continue after insulin infusion and removal of the CSII cannula

Solution for Problem 7. We believe that it is critical to preserve infusion site tissue integrity by controlling inflammation and infection both during and after insulin infusion. We use post-infusion anti-TRAP agents and delivery systems that control post-infusion tissue reactions and infections.

Methods to make cannulas and cannulas chronic insertion wounds more biocompatible and or prevent cannula infections/biofilms using liquid coating such as silicone, SLIPS and or Liquiglide with and without local drug delivery systems. Since poor cannula biocompatibility causes inflammation which insulin and its preservative can even further enhance, thereby decreasing CSII effectiveness, increasing cannula biocompatibility using liquid coating such as silicone, SLIPS and or Liquiglide with and without local drug delivery systems. Additionally, incorporating anti-microbial agents into the liquid coating such as silicone, SLIPS and or Liquiglide also prevent cannula related biofilms, infections and inflammation.

Removal of preservative and/or fibrils, from CSII systems; using drugs, factor and other agent to improve cannula compatibility.

### Pumps and Syringes Containing Filters/Absorbents.

FIGS. 72A and 72B show examples of insulin infusion systems incorporating in-line filters for removing preservatives and/or other TRAP-inducing agents.

FIGS. 72C and 72D show an example of a syringe incorporating a filter for removing preservatives and/or other TRAP-inducing agents.

Embodiments that Incorporate Collars at the Point of Insertion into the Skin

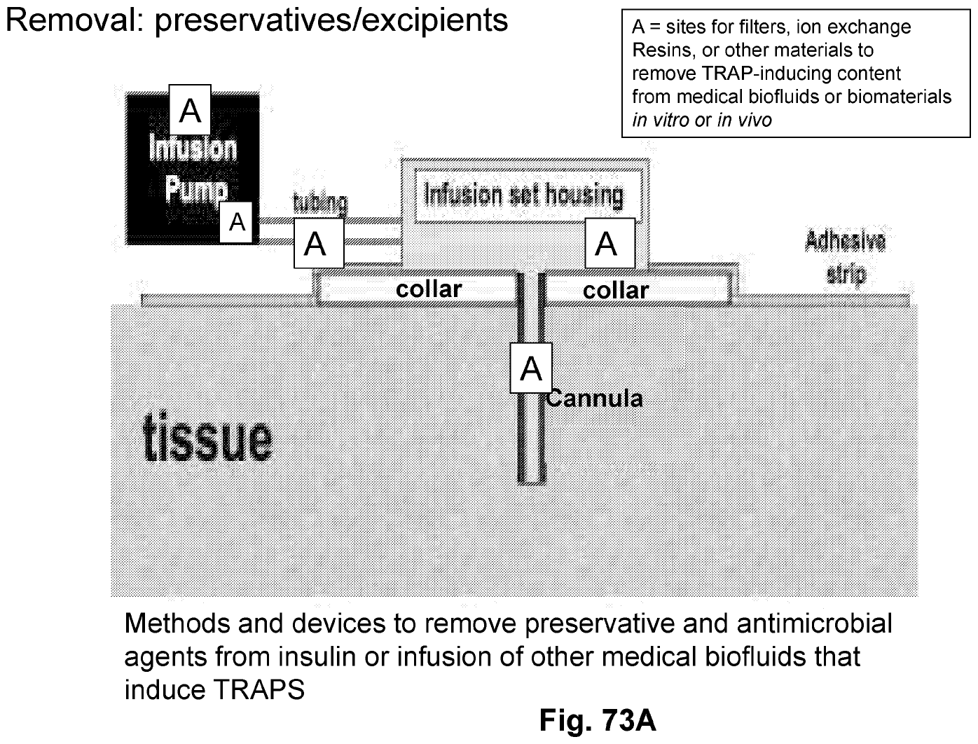

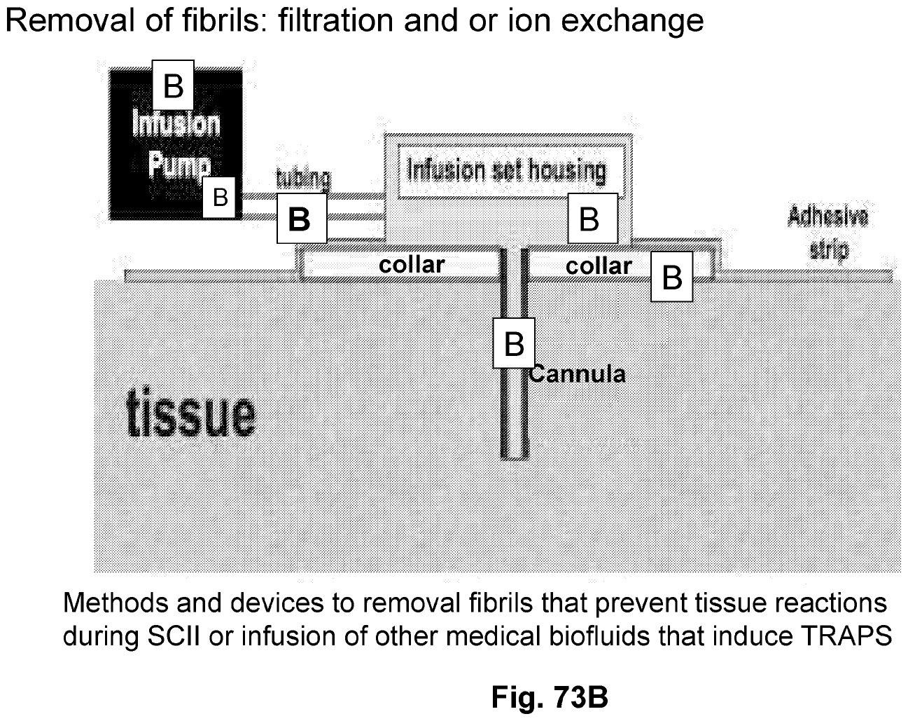

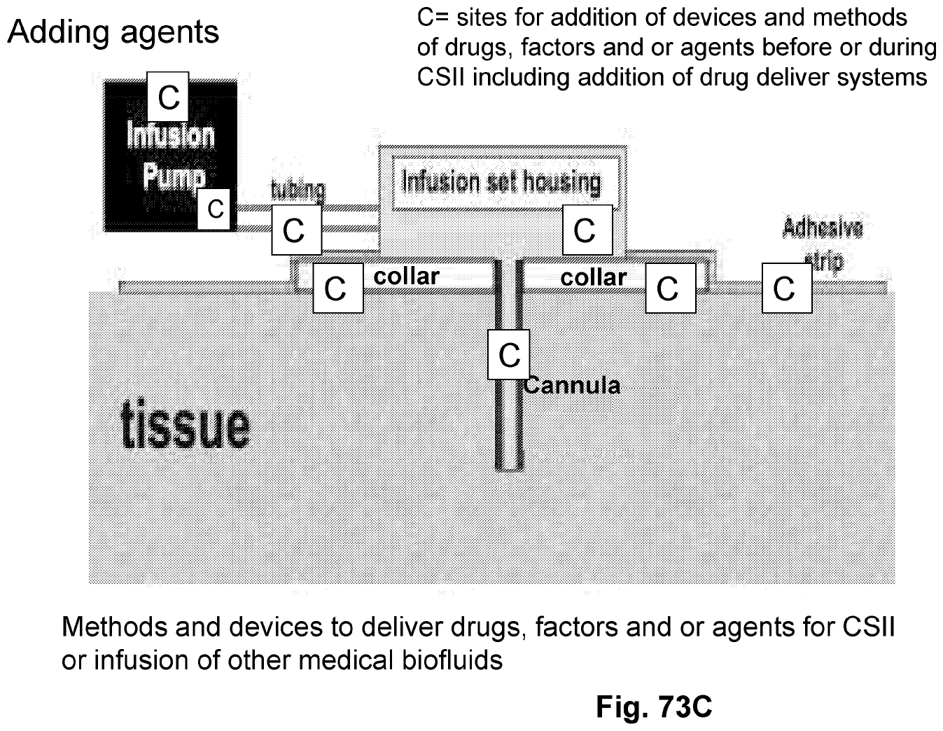

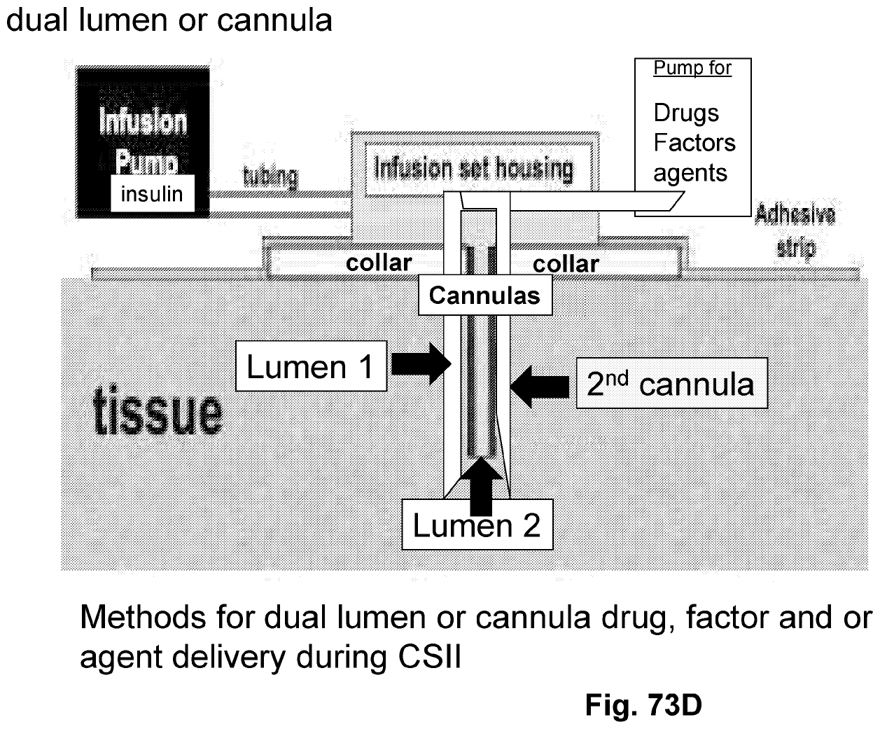

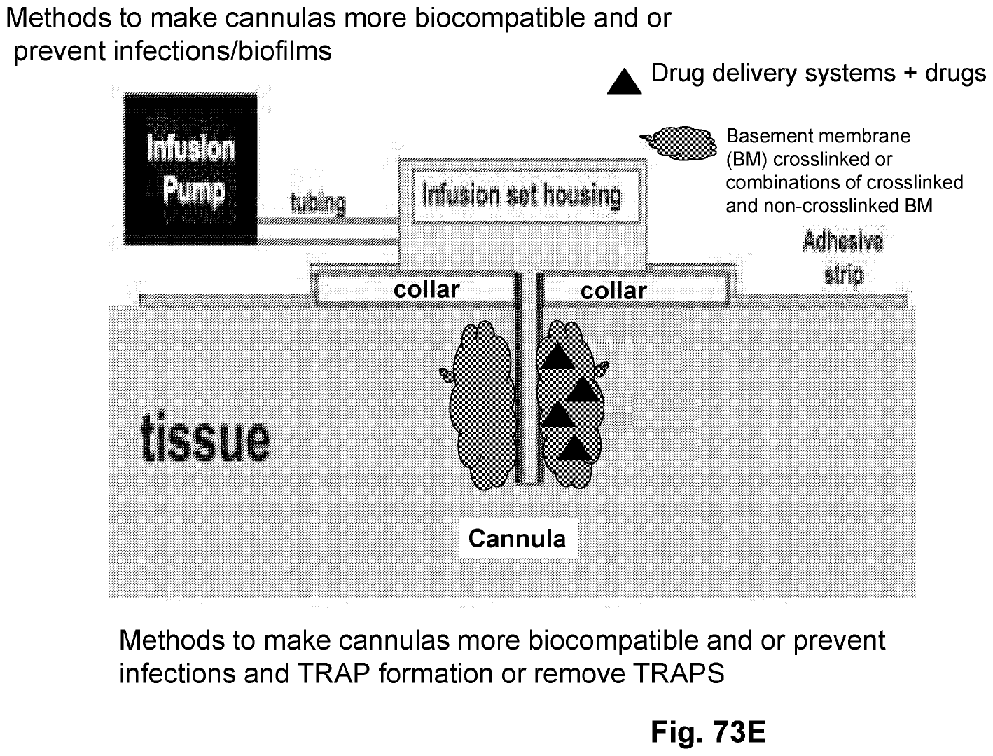

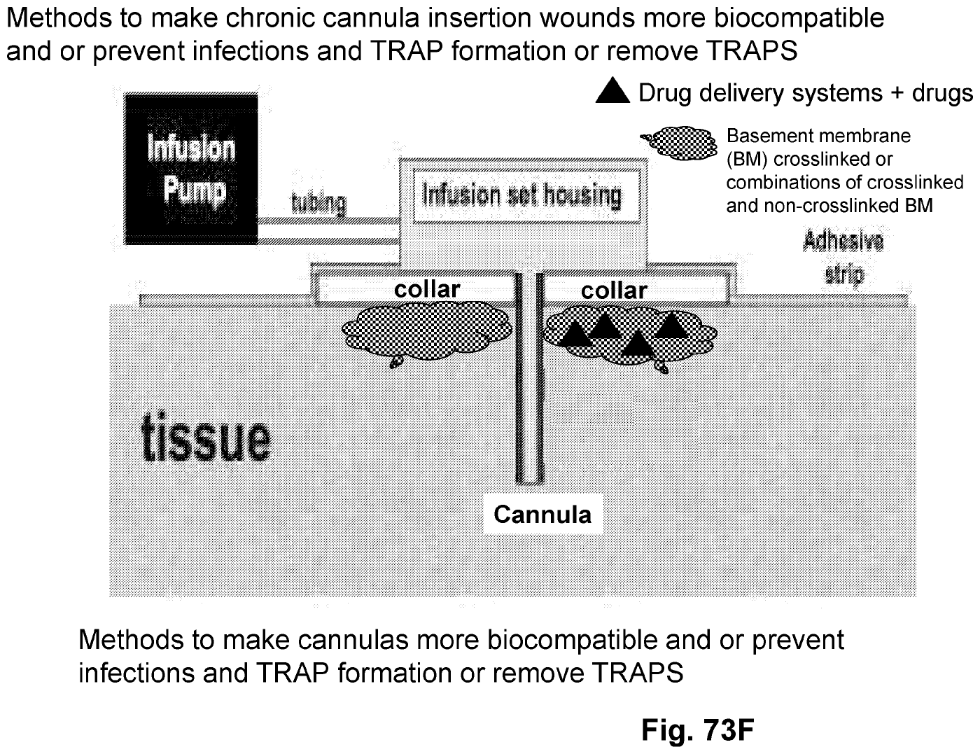

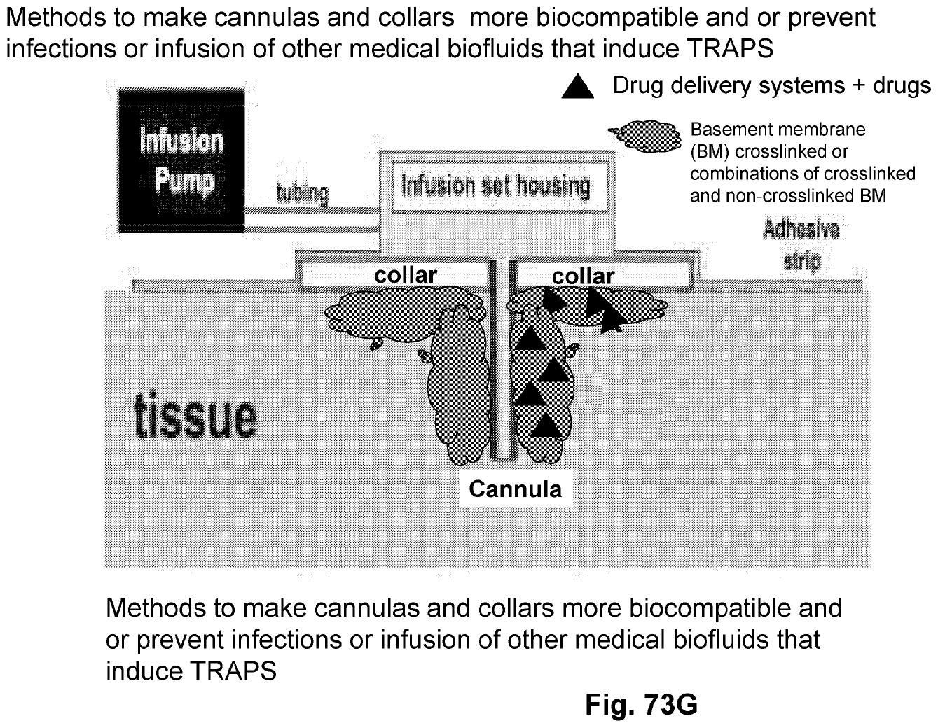

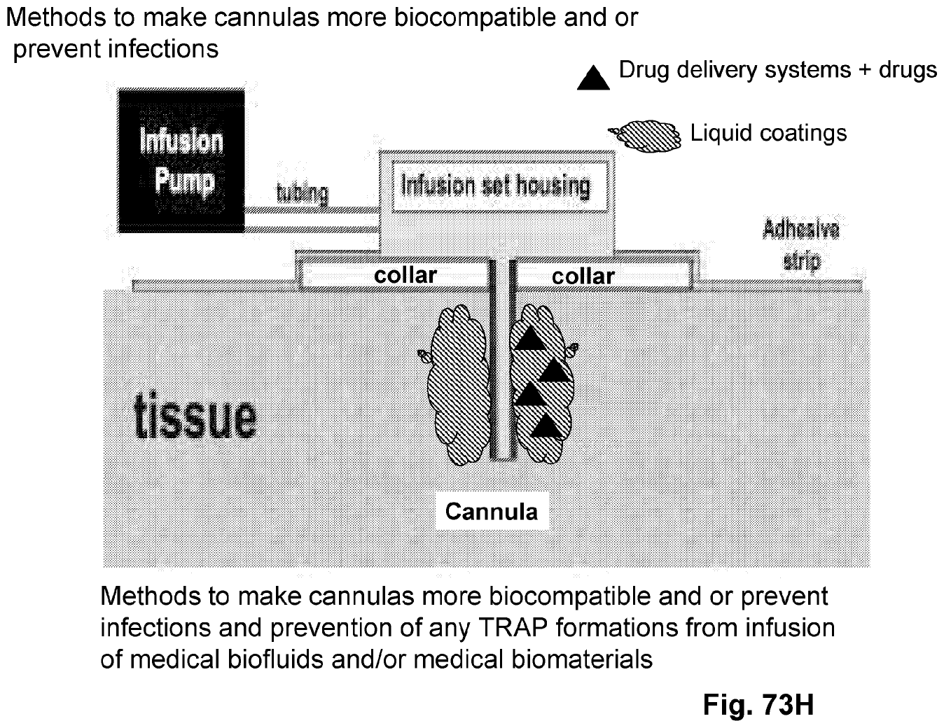

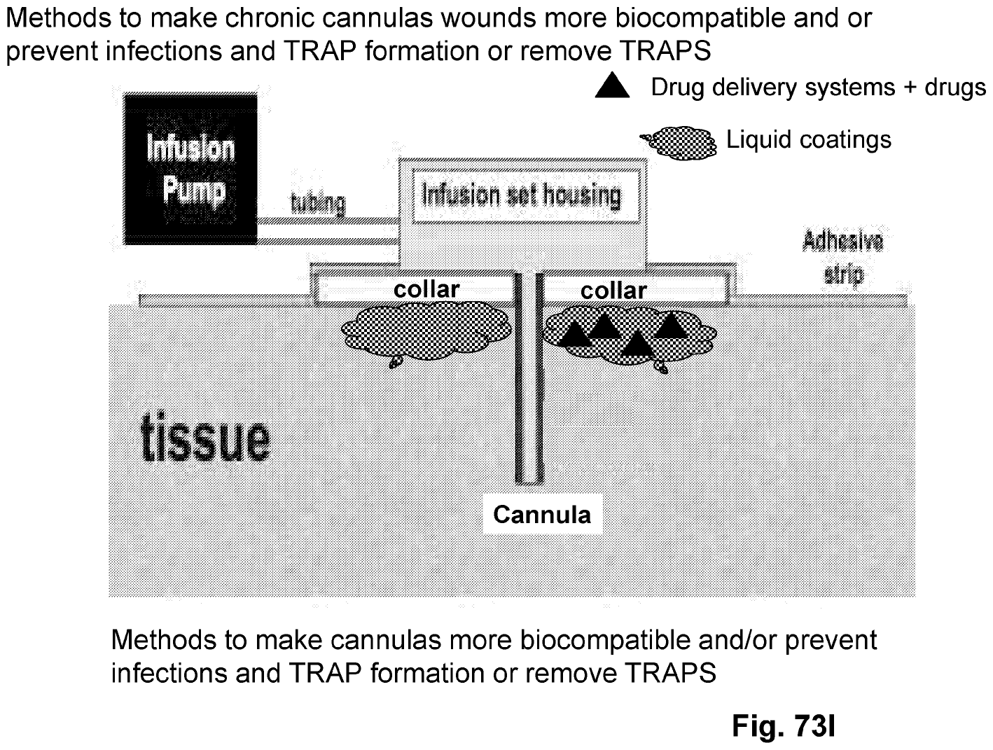

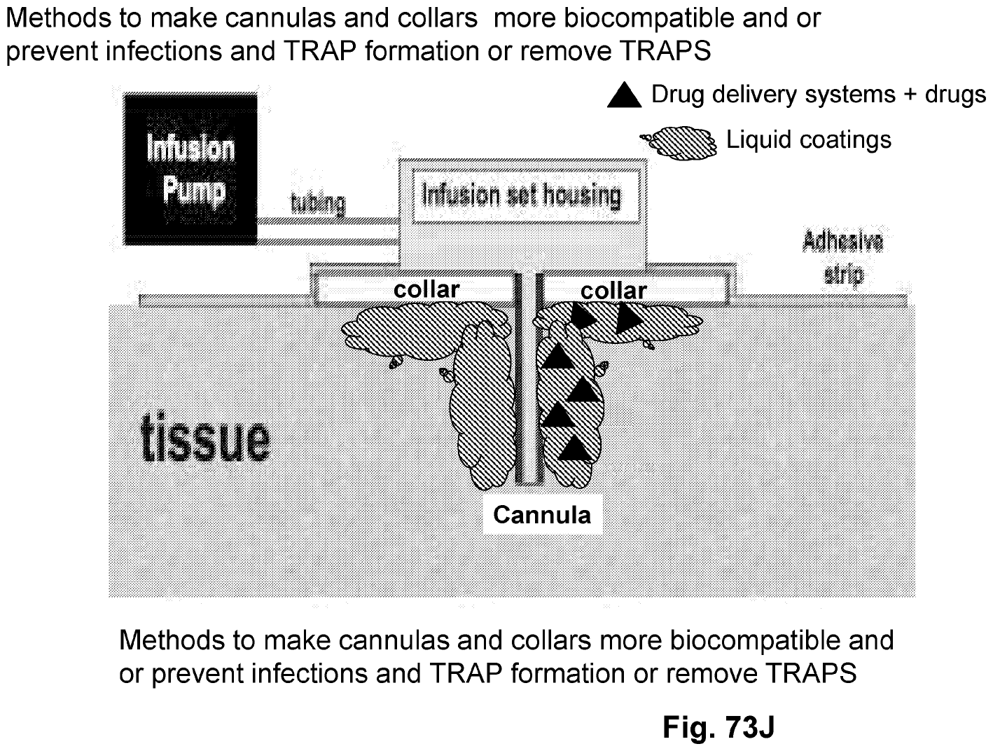

FIG. 73A shows a diagram of pump and infusion set indicating sites where preservatives or other TRAP-promoting compounds are removed by insertion of a removal or filtration system (designated as A with a white box in the diagram).

FIG. 73B shows a Diagram of pump and infusion set with indicating sites, designated as B, where anti-TRAP agents can be employed, which prevent and/or remove TRAP-inducing components during SCII or infusion of other medical bio-fluids. These TRAPS cause tissue destructive reactions.

FIG. 73C shows sites for addition of drugs, factors and or agents (including anti-TRAP agents) before or during CSII (designated C in white box) including addition of drug delivery systems. For example, adding drugs, factors and or agents to insulin formulations, or other medical bio-fluids, before or after introducing the insulin or other medical bio-fluids into the pump; introducing drugs, factors and or agents as the insulin/medical bio-fluids leaves the pump or inline release in the tubing; release of drugs, factors and or agents in the infusion housing or “cap” releasing drugs, factors and or agents from the cannula or cannulas in the tissue that prevent or reduce TRAP formation, or TRAP ability to trigger tissue reactions and/or infused medical bio-fluids.

FIG. 73D shows Dual lumen cannulas for separate delivery channels for insulin and other drugs, factors and or agents, including anti-TRAP agents, simultaneously at CSII infusion sites. This configuration prevents negative interactions between the insulin and drugs, factors and or agents use to control tissue reactions such as inflammation, fibrosis, and neovascularization during storage of the insulin or drugs, factors and or agents prior to infusion. This system can utilize a single pump or 2 separate pumps.

FIG. 73E depicts methods to make cannulas/medical devices or biomaterials more biocompatible and/or prevent cannula infections/biofilms using hydro-gels such as Basement membrane (BM) cross-linked or combinations of cross-linked and non-cross-linked BM with and without local drug delivery systems, including anti-TRAP agents/anti-TRAP agents. Since poor cannula biocompatibility causes inflammation, which insulin and its preservative can even further enhance, thereby decreasing CSII/medical bio-fluids effectiveness, increasing cannula biocompatibility using bio-hydrogels such as basement membrane coatings with or without drugs incorporated into the hydrogels also decrease TRAP-induced inflammation. Additionally, incorporating anti-microbial agents into the hydrogels also prevent cannula related biofilms, infections and inflammation, which can cause TRAP formation and tissue destruction in vivo.

FIG. 73F shows methods to make cannula chronic insertion wounds more biocompatible and or prevent infections and TRAP formation, as well as promote TRAP removal using collars of hydrogels, including extracellular matrices such as Basement membrane (BM) cross-linked or combinations of cross-linked and non-cross-linked BM with and without local drug delivery systems. Since chronic wounds result from extended cannula insertion, which in turn causes inflammation, which insulin, its preservative or other medical bio-fluids, and can even further enhance, thereby decreasing CSII medical bio-fluids effectiveness, increasing wound healing and biocompatibility using bio-hydrogels such as basement membrane coatings with anti-TRAP agents incorporated into the hydrogels decrease inflammation. Additionally, incorporating anti-microbial agents into the hydrogels also prevent cannula related biofilms, infections and TRAP formation and may help reduce TRAP related inflammation.

FIG. 73G shows methods to make cannulas and collars more biocompatible and/or prevent infections and/or trap formation/promote removal of TRAPS by combining the cannula biomaterials, coatings and cannula collars described above significantly prevent inflammation, infections prevent to formation and/or remove formed TRAPS when used in conjunction with each other, as well as with as without drugs, factors and/or agents, medical bio-material and medical bio-fluids.

FIG. 73H shows methods to make cannulas more biocompatible and or prevent cannula infections/biofilms/TRAP formation or removal of TRAPS using liquid coatings such as silicone, SLIPS and or Liquiglide with and without local drug delivery systems. Since poor cannula biocompatibility causes inflammation, which insulin and its preservative can even further enhance, thereby decreasing CSII or other medical bio-fluids effectiveness, increasing cannula biocompatibility using liquid coating such as silicone, SLIPS and or Liquiglide with and without local drug delivery systems. Additionally, incorporating anti-microbial agents into the liquid coatings such as silicone, SLIPS and/or Liquiglide also prevent cannula related biofilms, infections and inflammation.

FIG. 73I shows methods to make cannulas chronic insertion wounds more biocompatible and/or prevent cannula infections/biofilms using liquid coating such as silicone, SLIPS and or Liquiglide coating collars with and without local drug delivery systems. Since poor cannula biocompatibility causes inflammation, which insulin and its preservative can even further enhance, thereby decreasing CSII effectiveness, increasing cannula biocompatibility using liquid coatings such as silicone, SLIPS and or Liquiglide with and without local drug delivery systems. Additionally, incorporating anti-microbial and or anti-TRAP preventing and TRAP-reversing agents (e.g., DNases) into liquid coatings such as silicone, SLIPS and or Liquiglide also prevent cannula related biofilms, infections and trap-induced inflammation.

FIG. 73J shows methods of making cannulas and collars more biocompatible and/or prevent infections. Drug delivery systems containing drugs can be incorporated into liquid coatings to prevent TRAP formation and/or to remove TRAPs.

Use of Filters and/or Absorbing Materials at Other Locations in the CSII System to Remove Preservatives and/or Insulin Derived Fibrils

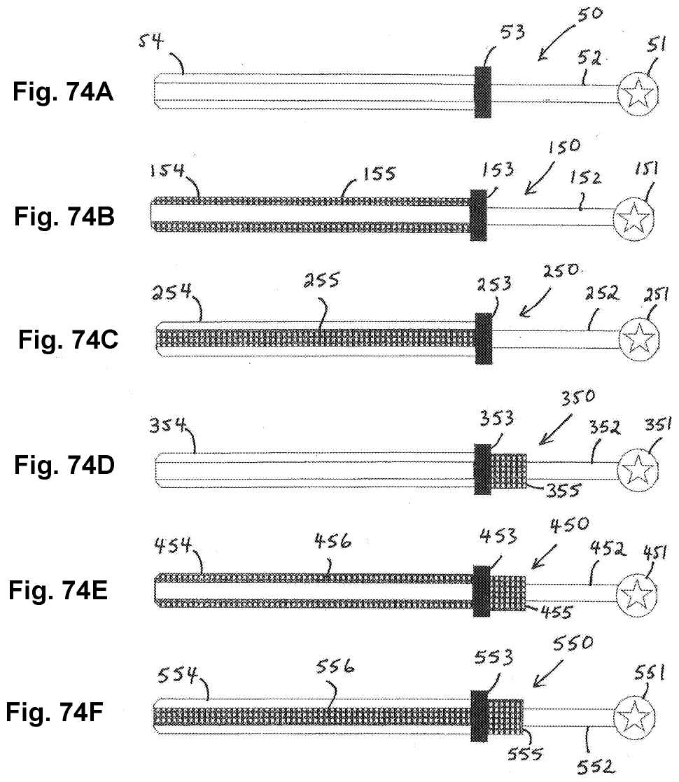

Control—FIG. 74A shows a conventional insulin delivery system. FIGS. 74B-74F schematically show filtration cannula and/or cannula prefilter systems to remove preservatives and/or insulin fibrils from insulin. In FIG. 74A the overall CSII system is generally designated as 50. The system 50 includes an insulin pump 51, which pumps insulin through an insulin delivery line 52. The insulin then enters an infusion housing 53 positioned between the delivery line 52 and a cannula 54. The cannula 54 is in direct contact with subcutaneous tissue in the body of a patient.

Coated Cannula—FIG. 74B shows an insulin delivery system in which the cannula is coated with, or made from, materials that can remove preservatives and/or fibrils or TRAP inducing agents from insulin or other medical bio-fluids formulations (or remove other substances from other liquids delivered in other types of delivery systems). In FIG. 74B the overall CSII/medical bio-fluids system is generally designated as 150. The system 150 includes an insulin/medical bio-fluids pump 151, which pumps insulin/medical bio-fluids through an insulin/medical bio-fluids delivery line 152. The insulin medical bio-fluids then enters an infusion housing 153 positioned between the delivery line 152 and a cannula 154. In the coated embodiment, the cannula is coated with a coating layer 155 of a filtration or absorbing material. In other cases, the cannula walls themselves are made from a filtration or absorbing material, which is a filtration system that removes preservatives and/or fibrils/or TRAP-inducing agents from the insulin/medical bio-fluids before the insulin/medical bio-fluids enters the patient's body. The use of the filtration system or absorbing material prevents or reduces TRAP-induced tissue inflammation, infection and loss of effective insulin medical bio-fluids delivery using a CSII or medical bio-fluids medical bio-fluids delivery system.

Filled Cannula—FIG. 74C shows an insulin medical bio-fluids delivery system in which the cannula is filled with a material that can remove preservatives and/or fibrils/TRAP-inducing agents from insulin/other medical bio-fluids formulations. In FIG. 74C the overall CSII/medical bio-fluids system is generally designated as 250. The system 250 includes an insulin/medical bio-fluids pump 251, which pumps insulin/medical bio-fluids through an insulin/medical bio-fluids delivery line 252. The insulin/medical bio-fluids then enters an infusion housing 253 positioned between the delivery line 252 and a cannula 254. The cannula 254 is filled with a material 255, which absorbs preservative and/or fibrils from the insulin/medical bio-fluids before the insulin medical bio-fluids enters the patient's body. The use of the absorbing material prevents or reduces tissue inflammation, infection or TRAP formation and promotes TRAP degradation (DNase) and loss of effective insulin/medical bio-fluids delivery using a CSII/medical bio-fluids system.

Modified Cannula Housing—FIG. 74D shows a system in which the cannula housing is filled with a material that can remove preservatives and/or fibrils and/or TRAP-inducing agents from insulin or other medical bio-fluids, or is made from a material that can remove preservatives and/or fibrils from insulin or other medical bio-fluids. In FIG. 74D the overall CSII/medical bio-fluids system is generally designated as 350. The system 350 includes an insulin/other medical bio-fluids pump 351, which pumps insulin/other medical bio-fluids through an insulin/medical bio-fluids delivery line 352. The insulin/biomedical fluids then enter an infusion housing 353 positioned between the delivery line 352 and a cannula 354. The infusion housing 353 contains a filtration or absorbing material 355, or is made from a filtration or absorbing material, which removes preservatives and/or fibrils and/or TRAP-inducing agents from the insulin or other medical bio-fluids.

Coated Cannula and Modified Cannula Housing—FIG. 74E shows a system which is a combination of the systems of FIGS. 74B and 74D. In FIG. 74E the overall CSII system is generally designated as 450. The system 450 includes an insulin/medical bio-fluids pump 451, which pumps insulin/other medical bio-fluids through an insulin/other medical bio-fluids delivery line 452. The insulin/biofluids then enters an infusion housing 453 positioned between the delivery line 452 and a cannula 454. In this embodiment, the infusion housing 453 contains a filtration or absorbing component 455, which removes one or more of preservatives and fibrils and/or TRAP-inducing agents. The walls of the cannula 454 are made of a filtration or absorbing material, which removes at least one of preservatives, fibrils or other TRAP-inducing agents. In some cases, component 455 removes preservatives and the cannula 454 wall material removes fibrils. In other cases, the component 455 removes fibrils and the cannula wall removes preservatives. Further embodiments, component 455 removes both preservatives and fibrils, while the cannula wall 454 removes either one of both of preservatives and fibrils. In other embodiments, the cannula wall removes both preservatives and fibrils while component 455 removes either preservatives or fibrils.

Filled Cannula and Modified Cannula Housing—FIG. 74F shows a system that is a combination of the systems of FIGS. 74C and 74D. In FIG. 74F the overall CSII system is generally designated as 550. The system 550 includes an insulin pump 551, which pumps insulin through an insulin delivery line 552. The insulin then enters an infusion housing 553 positioned between the delivery line 552 and a cannula 554. In this embodiment, the infusion housing 553 contains a filtration or absorbing component 555, which removes one or both of preservatives and fibrils. The cannula 554 is filled with a filtration or absorbing material 556, which absorbs preservative and/or fibrils from the insulin before the insulin enters the patient's body. In some cases, component 555 removes preservatives/TRAP-inducing agents and the material 556 inside the cannula 454 removes fibrils/TRAP-inducing agents. In other cases, the component 555 removes fibrils and material 556 removes preservatives and other TRAP-inducing agents. In further embodiments, component 555 removes both preservatives and fibrils, while material 556 removes either one or both of preservatives and fibrils. In other embodiments, material 556 removes both preservatives and fibrils and/or TRAP-inducing agents while component 555 removes either preservatives or fibrils or TRAP-inducing agents.

Use of Drugs, Factors and/or Other Agents to Improve Biocompatibility of Cannulas in Drug Delivery Systems, Including CSII

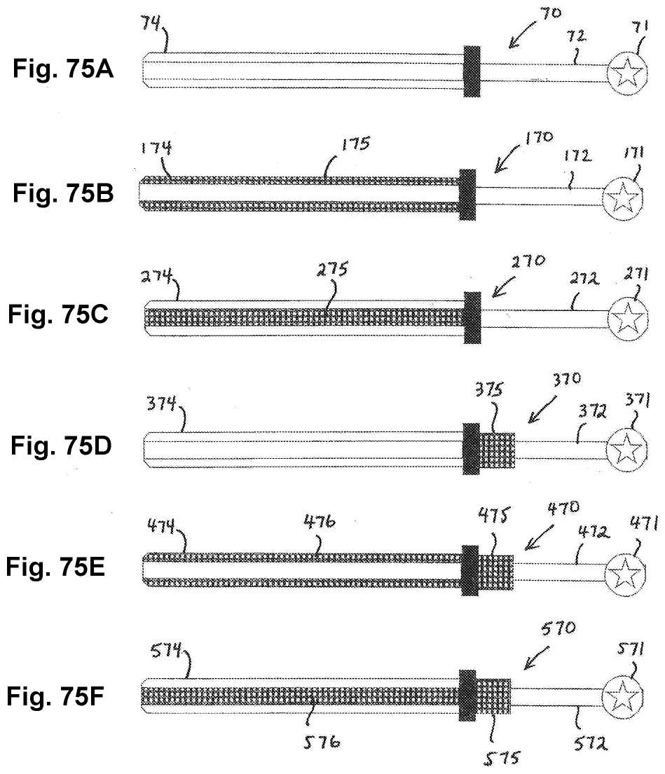

Control—FIG. 75A shows a conventional insulin delivery system. (FIGS. 75B-75F schematically show systems which incorporate drugs, factors and/or other agents, such as anti-TRAP agents, to improve biocompatibility of cannulas. In FIG. 75A the overall CSII/medical bio-fluids system is generally designated as 70. The system 70 includes an insulin/medical bio-fluids pump 71, which pumps insulin/medical bio-fluids through an insulin/medical bio-fluids delivery line 72. The insulin/medical bio-fluids then enters an infusion housing 73 positioned between the delivery line 72 and a cannula 74. The cannula 74 is in direct contact with the body of a patient.

Coated Cannula—FIG. 75B shows an insulin/medical bio-fluids delivery system in which the cannula is coated with materials that can deliver drugs, factors and/or other agents to improve insulin/other medical bio-fluids/preservative/cannula biocompatibility. In FIG. 75B the overall CSII/medical bio-fluids system is generally designated as 170. The system 170 includes an insulin medical bio-fluids pump 171, which pumps insulin/medical bio-fluids through an insulin/medical biofluids delivery line 172. The insulin/medical bio-fluids then enters an infusion housing 173 positioned between the delivery line 172 and a cannula 174. In the coated embodiment, the cannula is coated with a coating layer 155, which can deliver drugs, factors or agents that reduce TRAP-induced inflammation in the tissue that is in contact with, and surrounding, the cannula 174.

Filled Cannula—FIG. 75C shows an insulin delivery system in which the cannula is filled with a material that can deliver drugs, factors and/or other agents to improve insulin/medical bio-fluids/preservative/cannula biocompatibility. In FIG. 75C the overall CSII/medical bio-fluids system is generally designated as 270. The system 270 includes an insulin/medical bio-fluids pump 271, which pumps insulin through an insulin delivery line 272. The insulin then enters an infusion housing 273 positioned between the delivery line 272 and a cannula 274. The cannula 274 is filled with a component 275, which delivers drugs, factors or agents that reduce TRAP-induced inflammation in the tissue that is in contact with, and surrounding, the cannula 274. The use of the drugs, factors or agents prevents or reduces TRAP-induced tissue inflammation, infection and loss of effective insulin/medical bio-fluids delivery using a CSII/medical bio-fluids system.

Modified Cannula Housing—FIG. 75D shows a system in which the cannula housing is filled with a material that can deliver drugs, factors and/or other agents to improve insulin/medical bio-fluids/preservative/cannula biocompatibility. In FIG. 75D the overall CSII/medical bio-fluids system is generally designated as 370. The system 370 includes an insulin/medical bio-fluids pump 371, which pumps insulin/medical bio-fluids through an insulin/medical bio-fluids delivery line 372. The insulin/medical bio-fluids then enter an infusion housing 373 positioned between the delivery line 372 and a cannula 374. The infusion housing 373 contains a material 375 that can deliver drugs, factors and/or other agents, or is made from a material that can deliver drugs, factors or other agents that prevent TRAP formation or triggering TRAPS or dissolving performed TRAPS.

Coated Cannula and Modified Cannula Housing—FIG. 75E shows a system which is a combination of the systems of FIGS. 75B and 75D. In FIG. 75E the overall CSII system is generally designated as 470. The system 470 includes an insulin pump 471, which pumps insulin through an insulin delivery line 472. The insulin then enters an infusion housing 473 positioned between the delivery line 472 and a cannula 474. In this embodiment, the infusion housing 473 contains a component 475, which delivers drugs, factor or other agents that promote biocompatibility. The wall 476 of the cannula 474 has an outer coating 478 of this type of material. In some cases, component 475 delivers one type of substance and the coating 478 delivers another type of substance. In other cases, both component 475 and the coating 478 of the cannula 474 deliver the same substances.

Filled Cannula and Modified Cannula Housing—FIG. 75F shows a system that is a combination of the systems of FIGS. 75C and 75D. In FIG. 75F the overall CSII/medical bio-fluids system is generally designated as 570. The system 570 includes an insulin/medical bio-fluids pump 571, which pumps insulin/medical bio-fluids through an insulin/medical bio-fluids delivery line 572. The insulin then enters an infusion housing 573 positioned between the delivery line 572 and a cannula 574. In this embodiment, the infusion housing 573 contains a component 575, which delivers drugs, factor or other agents that promote biocompatibility including preventing and removing TRAPS. The cannula 574 is filled with a material 576, which delivers drugs, factor or other agents that promote biocompatibility including preventing and removing TRAPS. In some cases, component 575 delivers one type of substance and the material 576 delivers another type of substance. In other cases, both component 575 and the material 576 deliver the same substances.

Removal of Fibrils and/or Preservatives and/or TRAP-Inducing Agents from Insulin/Medical Bio-Fluids Delivered by a Syringe

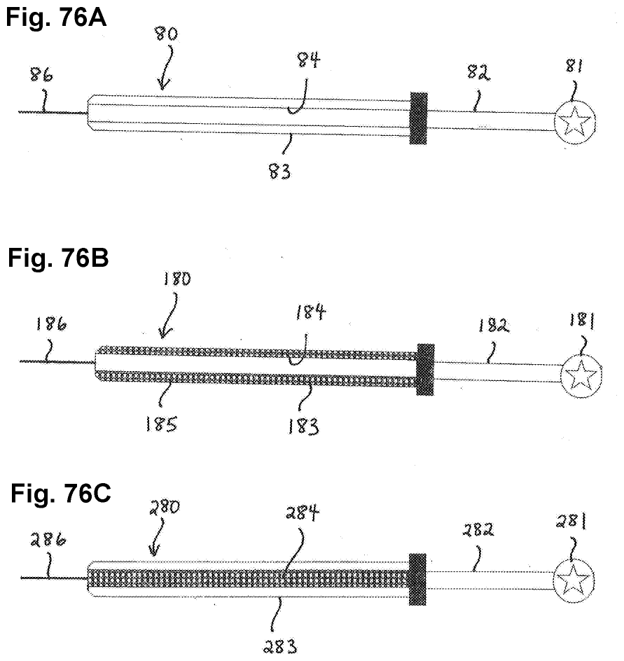

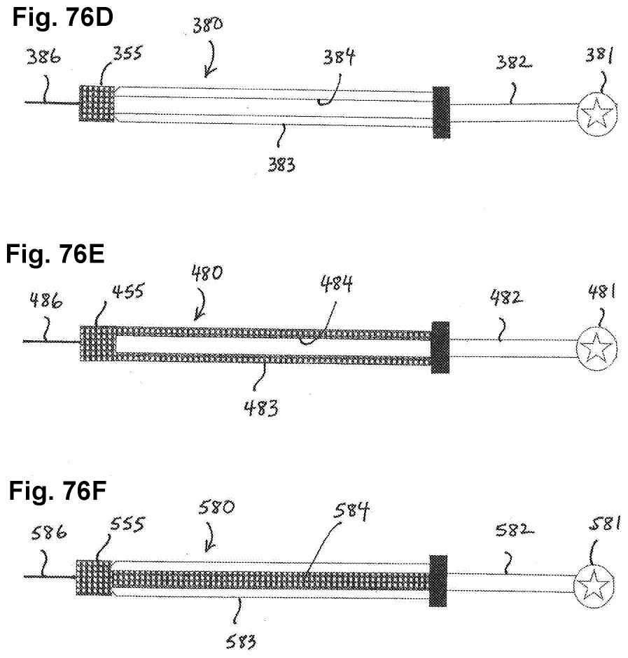

Control—FIG. 76A shows a conventional syringe-type insulin/medical bio-fluids delivery system. FIGS. 76B-76F schematically show syringe chamber and/or plunger sleeve systems to remove preservatives and/or insulin fibrils/TRAP-inducing agents from insulin/medical bio-fluids. In FIG. 76A the overall insulin/medical bio-fluids delivery system is generally designated as 80. The system 80 includes a plunger cap 81, a plunger sleeve 82, a syringe housing 83, a syringe chamber 84 and a needle 86. The insulin/medical bio-fluids enter the patient through the outer end of the needle 86. At least a portion of the needle 86 is in direct contact with subcutaneous tissue in the body of a patient during insulin/medical bio-fluids delivery.

Coated Syringe Chamber—FIG. 76B shows an insulin/medical bio-fluids delivery system in which the inner wall of the syringe housing is coated with, or made from, materials that can remove preservatives and/or fibrils from insulin/medical bio-fluids formulations (or other substances from other types of liquids) or TRAP-inducing agents. In FIG. 76B the overall syringe-type insulin/medical bio-fluids deliver system is generally designated as 180. The system 180 includes a plunger cap 181, a plunger sleeve 182, a syringe housing 183, a syringe chamber 184 and a needle 186. The insulin/medical bio-fluids in the syringe chamber 184 enter the patient's body through the needle 186. In the coated embodiment, the inner wall 187 of the syringe housing, that is, the tubular wall defining the syringe chamber 184, is coated with a coating layer 185 which can remove preservatives and/or fibrils/TRAP-inducing agents. In other cases, the syringe inner chamber wall 187 itself is made from a system that removes preservatives and/or fibrils from the insulin and/or TRAP-inducing agents from insulin or other medical bio-fluids enter the patient's body. The use of the filtration system or absorbing TRAP-inducing agents and material to prevent or reduces TRAP-induced tissue inflammation, infection and loss of effective insulin/medical bio-fluids delivery using a syringe-type insulin/medical bio-fluids delivery system.

Filled Syringe Chamber—FIG. 76C shows an insulin/medical bio-fluids delivery system in which the syringe chamber contains a porous material that can remove preservatives and/or fibrils of TRAP-inducing agents from insulin/medical bio-fluids formulations. In FIG. 76C the overall syringe-type insulin/medical bio-fluids deliver system is generally designated as 280. The system 280 includes a plunger cap 281, a plunger sleeve 282, a syringe housing 283, a syringe chamber 284 and a needle 286. The insulin/medical bio-fluids in the syringe chamber 284 enters the patient's body through the needle 286. The component 285 is formed from a material that removes preservatives and/or fibrils/or TRAP-inducing agents from the insulin/medical bio-fluids is contained within the syringe chamber 284.

End-Modified Syringe Chamber—FIG. 76D shows an insulin/medical bio-fluids delivery system in which the downstream end of the syringe housing is coated with, or made from, materials that can remove preservatives and/or fibrils from insulin/medical bio-fluids formulations. In FIG. 76D the overall syringe-type insulin/medical bio-fluids deliver system is generally designated as 380. The system 380 includes a plunger cap 81, a plunger sleeve 382, a syringe housing 853, a syringe chamber 384 and a needle 386. The insulin/medical bio-fluids in the syringe chamber 384 enters the patient's body through the needle 386. At the downstream end of the syringe chamber, a filter, absorbing material, or other component 385 is incorporated in order to remove preservatives and/or fibrils/or TRAP-inducing agents before the insulin/medical bio-fluids enter a patient's body. The use of the filtration system or absorbing material prevents or reduces tissue inflammation, infection and loss of effective insulin/medical bio-fluids delivery using a syringe-type insulin/medical bio-fluids delivery system.

Coated and End-Modified Syringe Chamber—FIG. 76E shows an insulin/medical bio-fluids delivery system that contains a combination of the elements shown in FIGS. 76B and 76G. In FIG. 76H the overall syringe-type insulin/medical bio-fluids deliver system is generally designated as 480. The system 480 includes a plunger cap 481, a plunger sleeve 482, a syringe housing 483, a syringe chamber 484 and a needle 486. The component that removes preservatives and/or fibrils/or TRAP-inducing agents is designated as 485.

Filled and End-Modified Syringe Chamber—FIG. 58F shows an insulin/medical bio-fluids delivery system that contains a combination of the elements shown in FIGS. 60C and 60G. In FIG. 60FI the overall syringe-type insulin/medical bio-fluids deliver system is generally designated as 550. The system 550 includes a plunger cap 581, a plunger sleeve 582, a syringe housing 583, a syringe chamber 584 and a needle 586. The components that remove preservatives and/or fibrils/TRAP-inducing agents is designated as 585.

The embodiments shown in FIGS. 76B-76F can be revised to incorporate further drugs, factors, and/or agents in place of, or in addition to, anti-TRAP/anti-TRAP agents.

In some embodiments described herein an antimicrobial agent is combined with the anti-TRAP agent. A non-limiting example of when an antimicrobial agent also is useful is for wounds such as diabetic foot ulcers which have infection and inflammation.

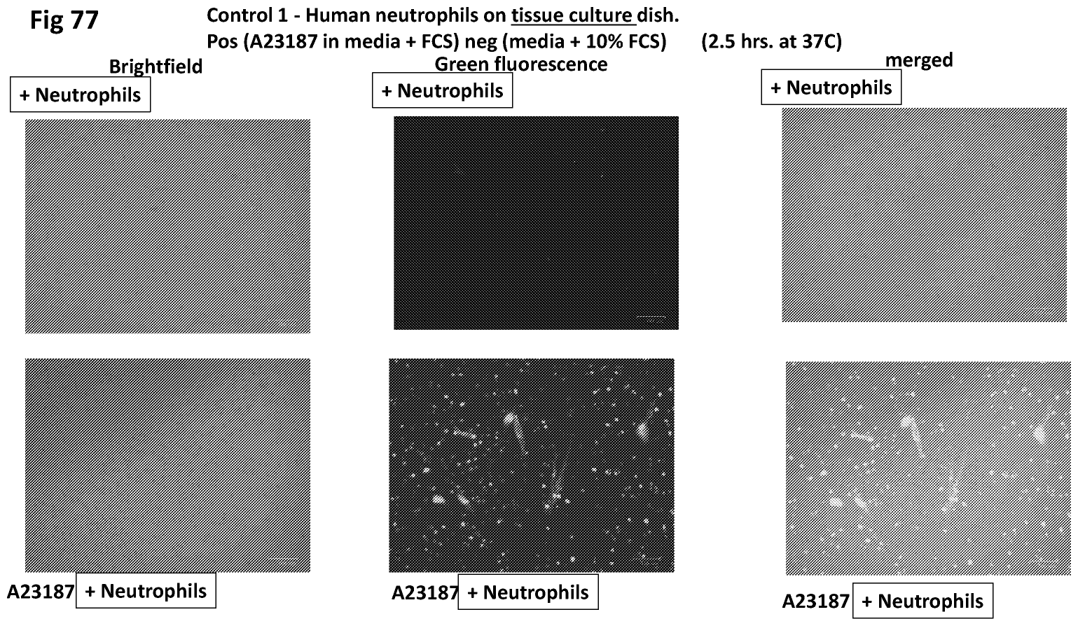

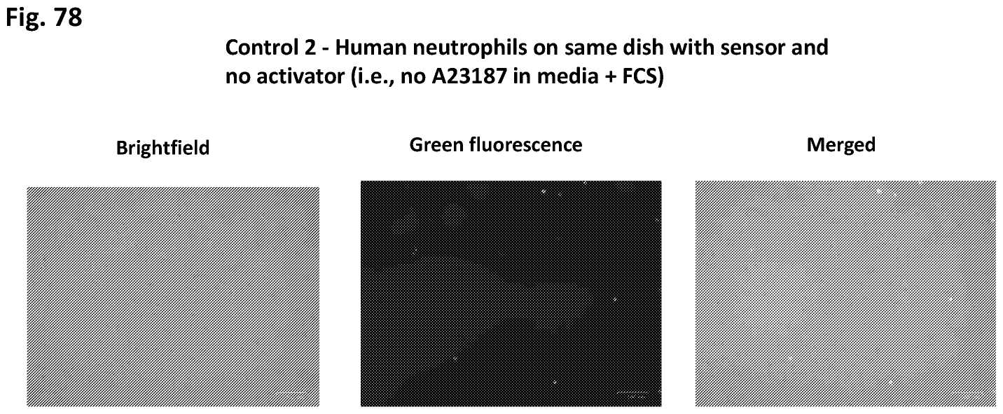

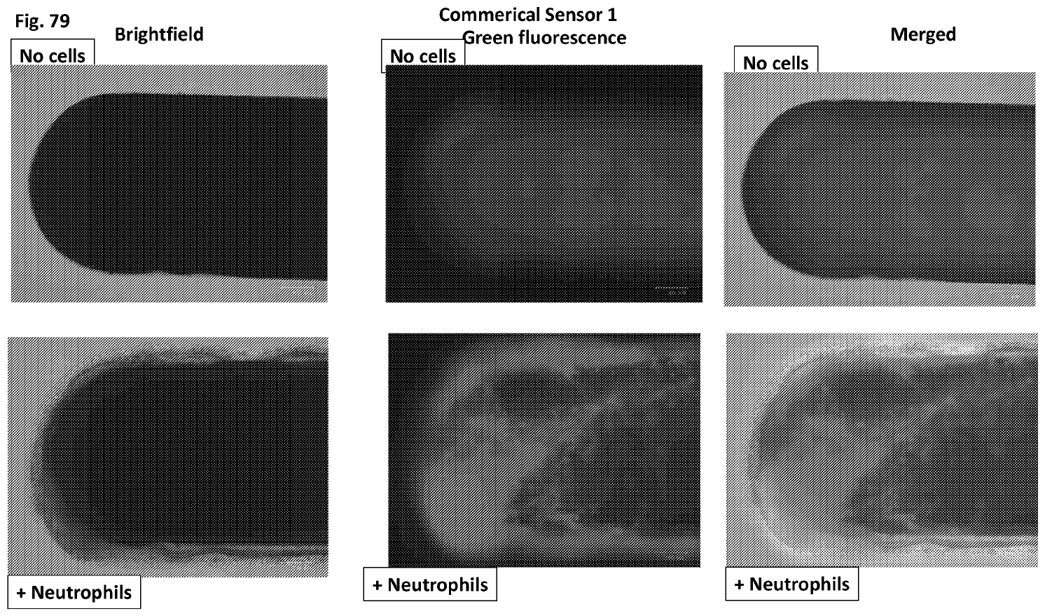

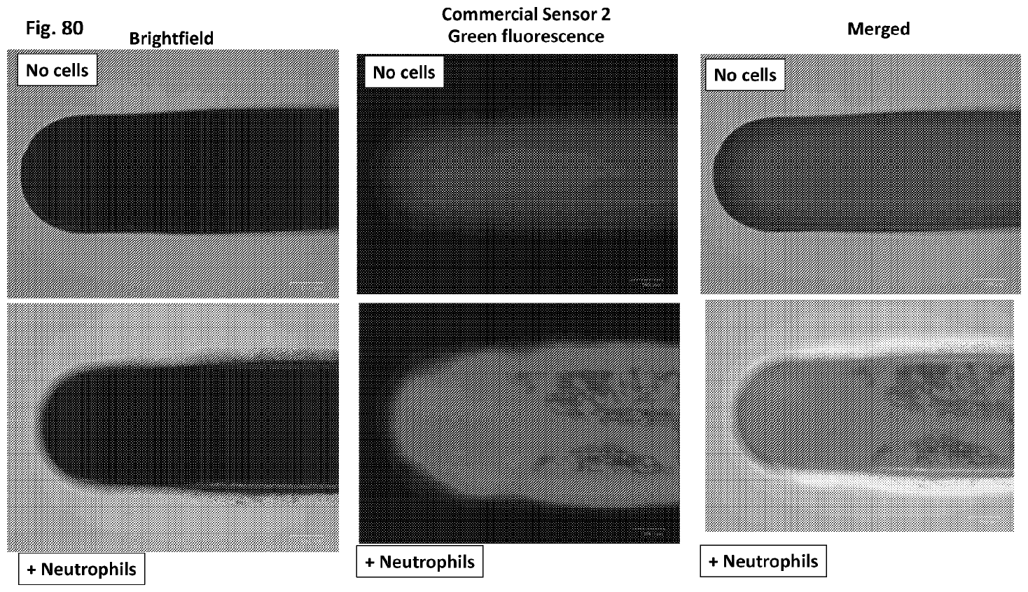

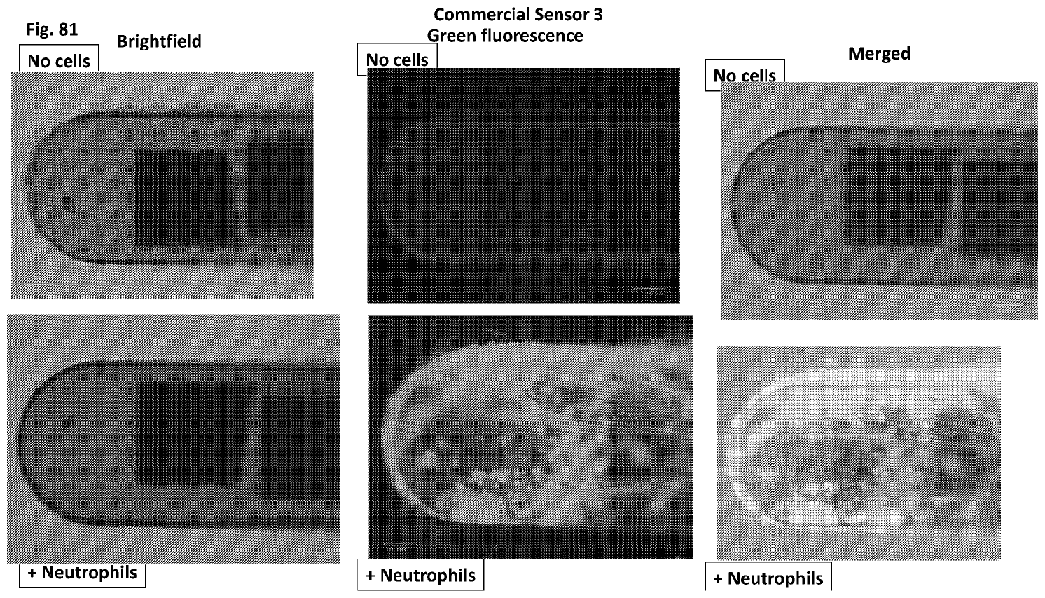

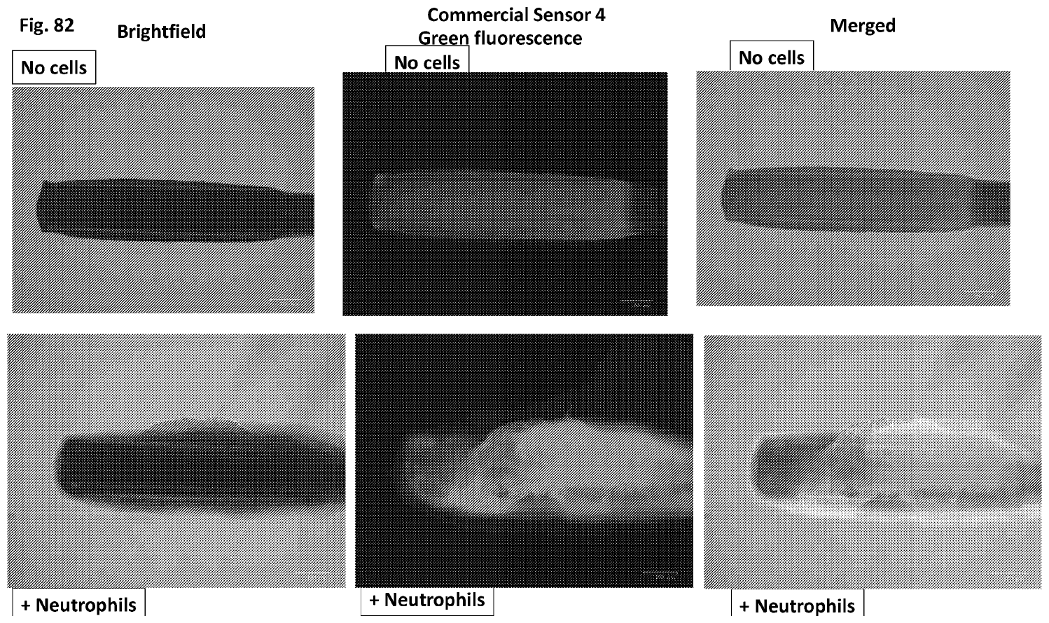

FIGS. 77-82 show the effects of the presence of glucose sensors on triggering human adult blood neutrophils to produce TRAPs i.e. Nets. In these examples, human blood neutrophils were placed in a culture dish+/−sensors in vitro. The photos show sections of the sensors, the sensor sections are letter A, B, C, etc, with section A being the sensor tip, which measures blood glucose levels in the deep skin layers, to the upper section of the sensor (higher letters) and there the upper layer of the skin (i.e. near the outside of the skin i.e. the air. FIG. 77 shows photos of neutrophil in a culture dish for a controls (negative controls (77 a-c) and positive control of A23187 treated neutrophils (77 d-f). Component A23187 is a commercially available inducer of TRAPS. FCS is fetal calf serum nutrition for neutrophils in vitro. FIG. 78abc shows photos of neutrophils in a separate culture dish with an inserted glucose sensor, but the cells are distant from the sensor. Brightfield refers to the use of non-fluorescent white light (78a). This data support that sensors do not release toxic/trap inducing substances into the media with the neutrophils. In FIG. 79, neutrophils were added to tissue culture dishes that contained a sensor (commercial type #1) to see if the sensor could directly trigger neutrophil death and TRAP release which we detect with fluorescent dyes like sytox green. FIG. 79a-c had no neutrophils added to the sensors. In FIG. 79d-f neutrophils were added. The sytox green binds to DNA, and fluoresces green in color photo, or white in black and white photos. The large white cables and clouds in FIGS. 79e and f are indicative of presence of TRAPS. White dots are dying and dead cells are beginning to release the DNA TRAPs. “Merged” refers to photos in which the non-fluorescent (brightfield) and fluorescent photos are merged into a single photo FIG. 79c. Additional study studies with difference commercial sensors (FIGS. 80-82) gave additional results indicating that commercial glucose sensor all trigger neutrophil cell death associated with DNA release i.e. TRAP formation in vitro.

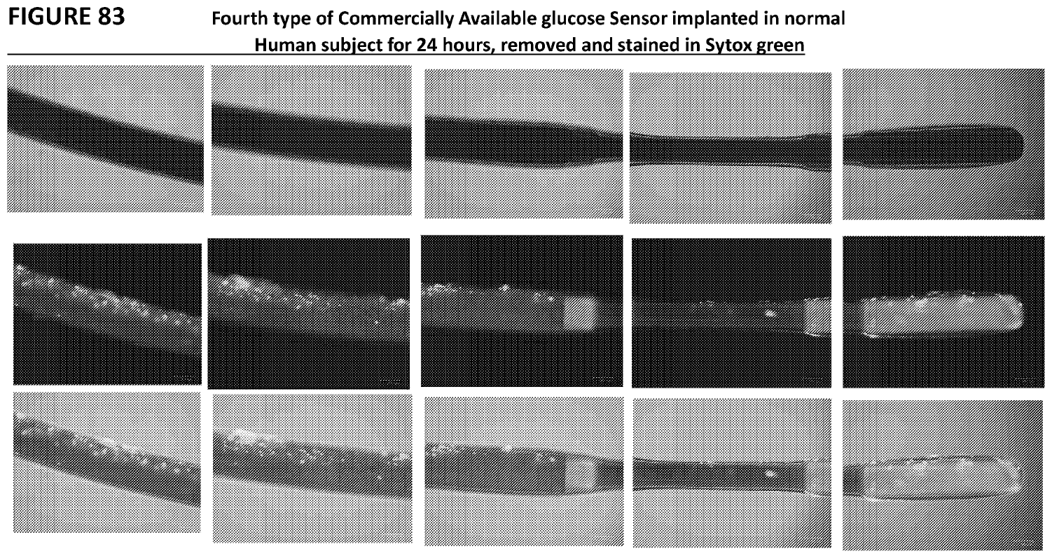

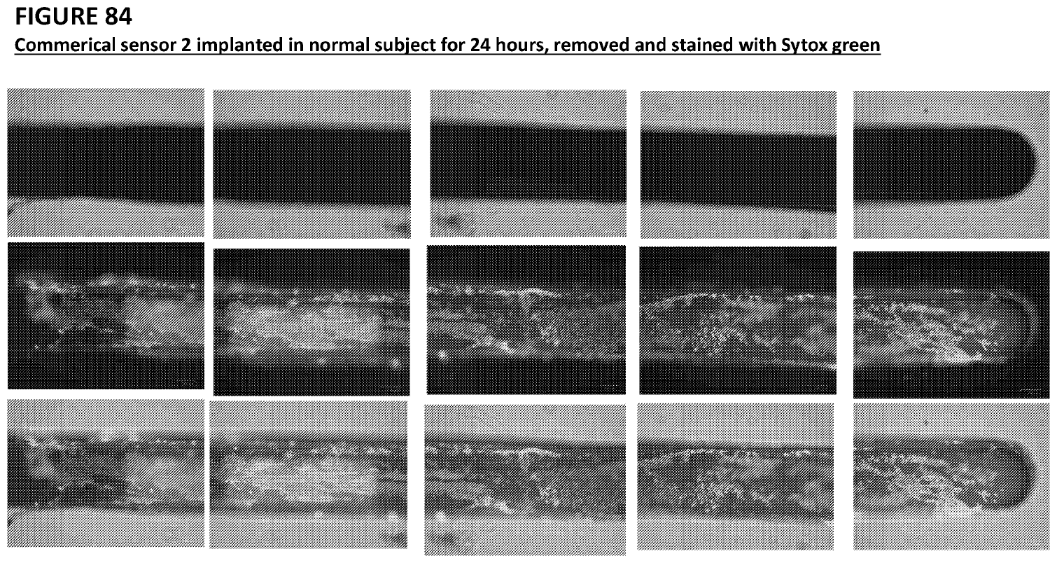

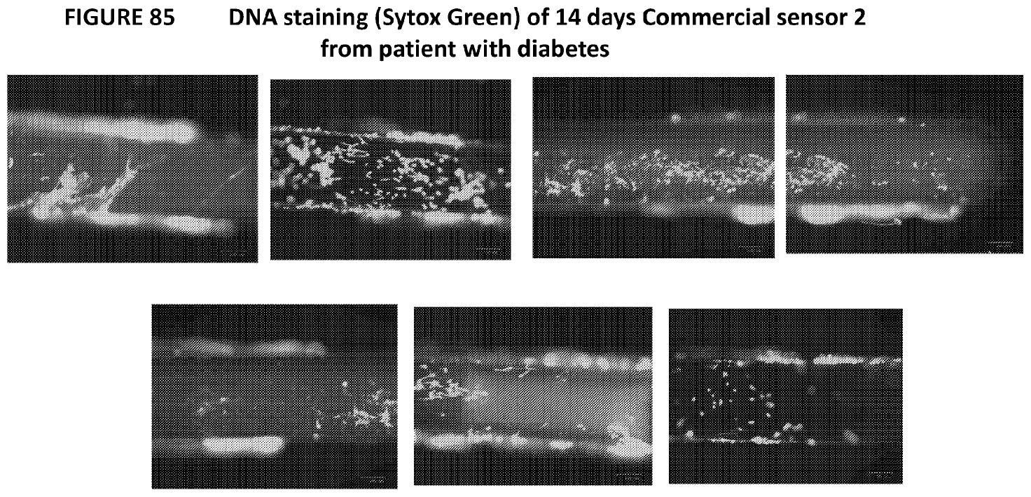

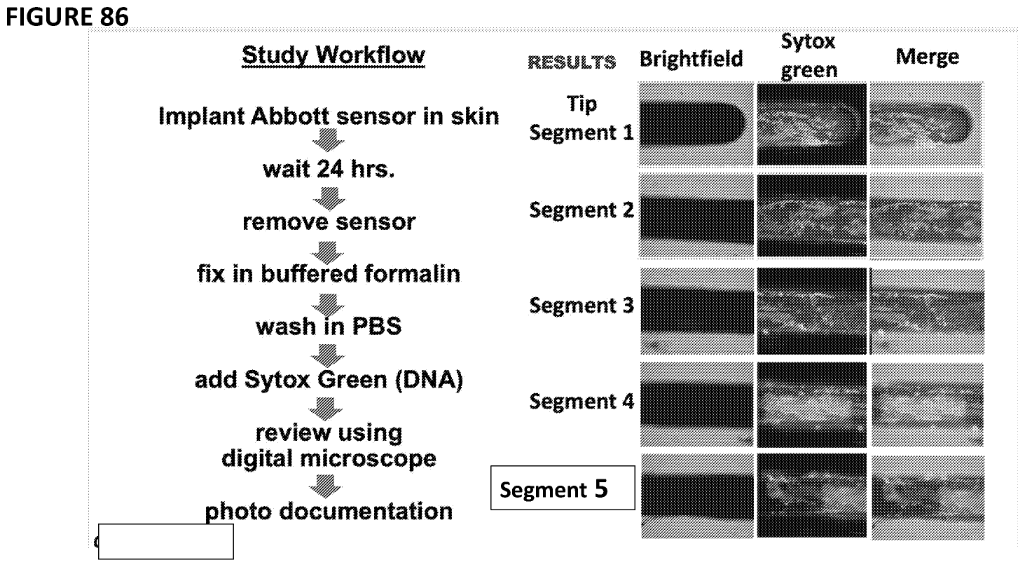

Our next question was do traps form in sensors implanted in normal human subjects' skin, i.e. in vivo?FIGS. 83-84 show in vivo human data for sensors implanted for 24 hours, then removed and stained to detect TRAPS, including NETS. FIG. 85 shows in vivo human data for a sensor implanted for 14 days in a patient with diabetes. FIG. 86 shows the results of an in vivo study in which in Abbott sensor was implanted in a normal subject for 24 hours, and then tested. The methodology for the experiments illustrated in FIGS. 83-86 (using various commercial sensors) is shown on the left side of FIG. 86. The photos show a significant presence of NETS/TRAPS on the surface of the sensors implanted in normal subjects and a patient with diabetes,

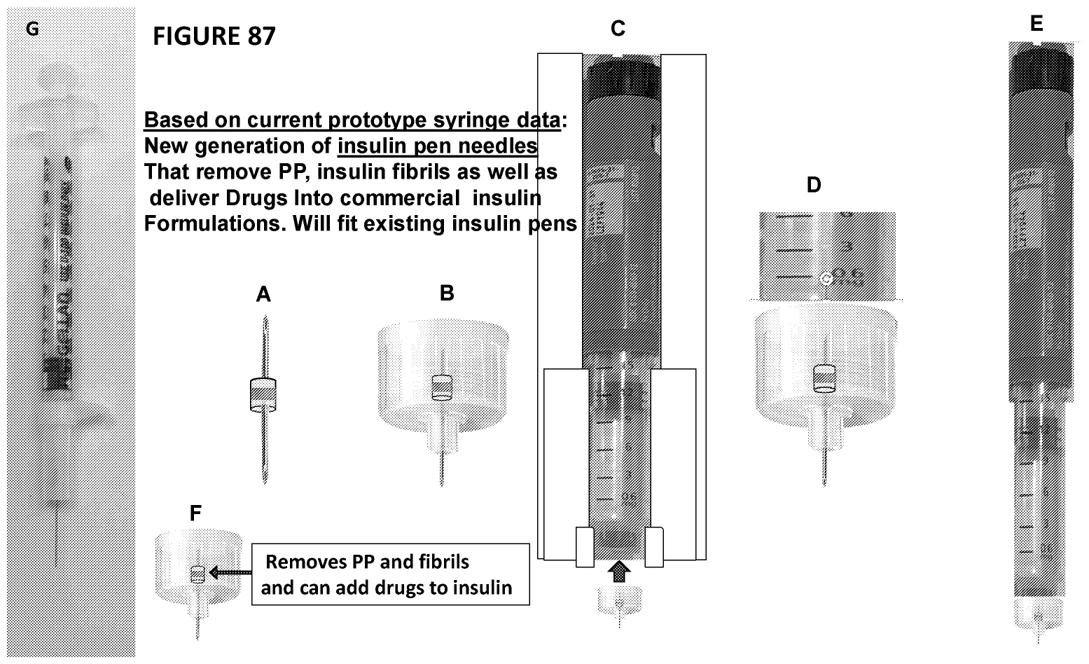

FIG. 87 shows an insulin pen and needle according to certain embodiments. This is a device to remove PP and fibrils from commercial insulins that cause nets and traps to form at sites if drug injections. For this application a resin such a zeolite or cyclodextrins can be incorporated into a disposable needle housing (e.g. insulin pens) used for drug injection (87 e). As the drug flows through the needle from the drug tank in the pen to the injection site, it can remove toxic agent in the drug that cause trap formation in injected tissue during treatment of patients. Also shows that this device can release/deliver additions drugs that can block net/trap formation and or drugs that can block or blunt the tissue damage induced by nets/traps at insulin infusion sites (FIG. 87 f).

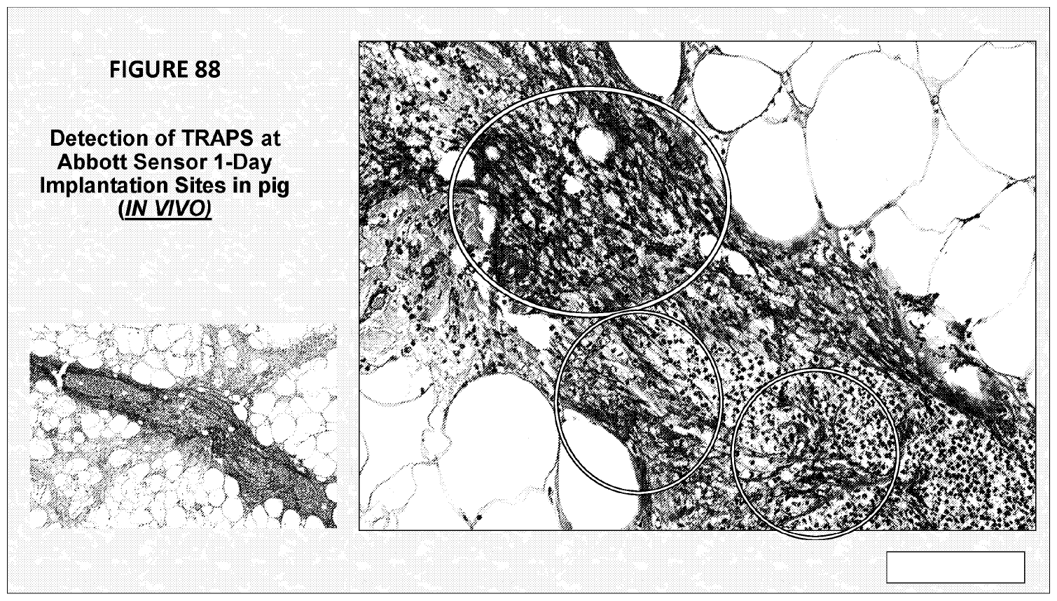

FIG. 88 shows photos of in vivo pig tissue section after insertion of a glucose sensor which remains in place for 24 hours. The photos show TRAP formation next to the sensor after staining with hematoxylin dye a DNA staining dye. The black “rivers’ in the tissue sections are TRAPs (DNA). The large white components are fat cells and the small dark dots are cells.

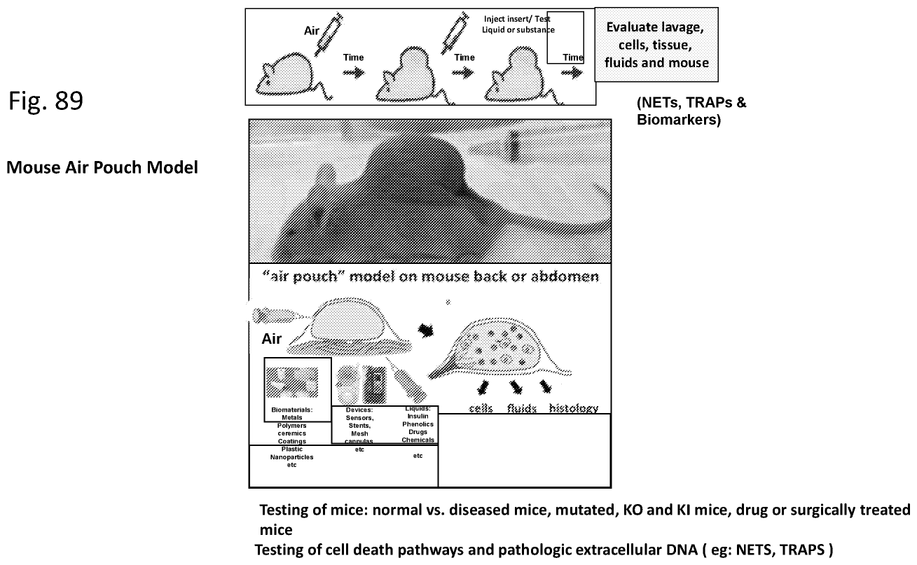

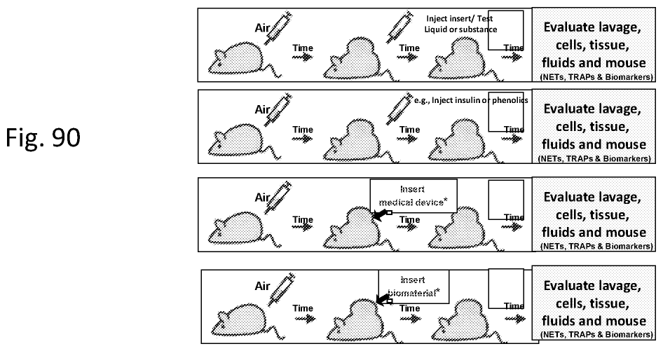

FIGS. 89-90 illustrate the use of an air pouch model for testing for the inducement and/or inhibition as well as quantification of TRAPs and tissue reactions in living tissue using Air pouch models. In a first embodiment, liquids and drugs are tested, such as medical fluids, including insulin, inducers of TRAPS, inhibitors of TRAPS, and molecular drugs such as those that include DNA and/or RNA. In a second embodiment, devices are tested to determine whether they induce or inhibit the formation of TRAPS/NETS, including implantable and non-implantable medical devices, including but limited to sensors, stents, mesh cannulas and biomaterials. A third embodiment involves the testing of biomaterials such as coatings on implantable and non-implantable medical devices (sensors, stents, mesh, cannulas, and biomaterials) to determine whether they inhibit or promote the formation of TRAPS/NETS. A fourth embodiment uses the air pouch mouse model for the testing of drug delivery platforms, including coatings for implantable and non-implantable medical devices (sensors, stents mesh cannulas, biomaterials, and determining whether they inhibit or promote the formation of TRAPS/NETS.

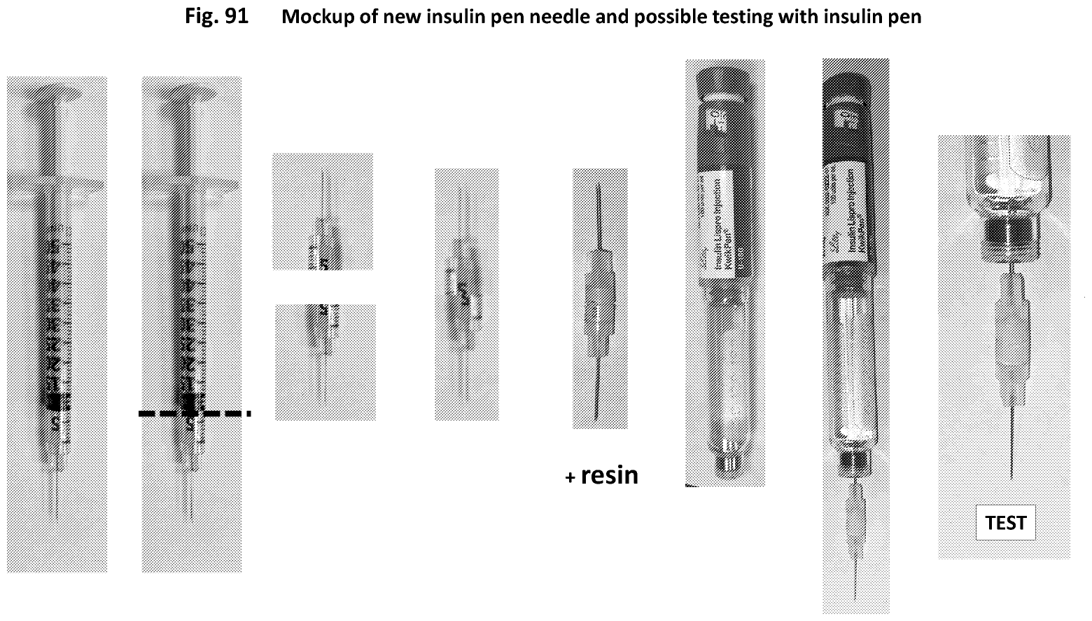

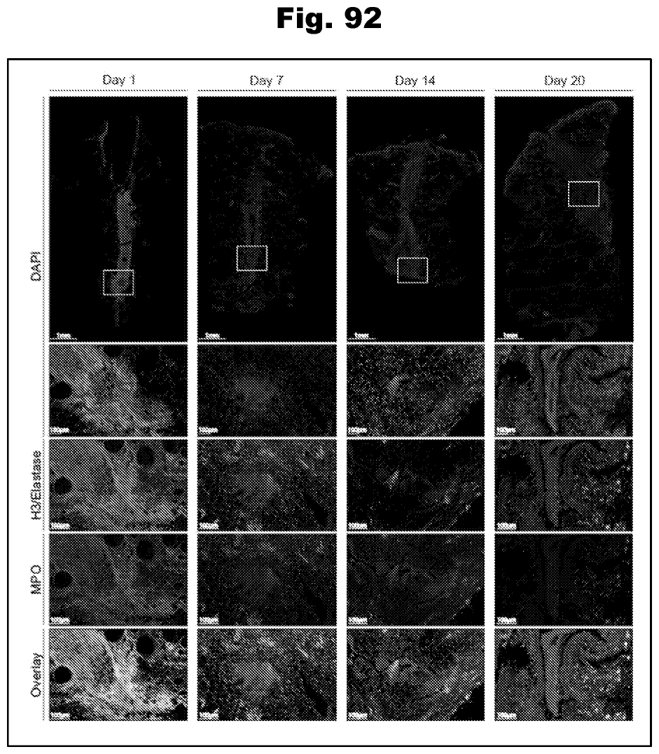

FIG. 91 schematically shows a prototype of a revised insulin pen in accordance with embodiments disclosed herein. For example, the filtration mechanism described removes toxic substances from the commercial injection systems prior to introduction of insulin or another liquid drug into tissue. Otherwise, the toxic substances may induce TRAP formation after the liquid is introduced into the tissue. This device also can be used to introduce anti-TRAP agents separately from, of currently with filtration of toxic substances. This system also can be applied in connection with infusion devices such as that shown in FIGS. 72A and 72B.

FIG. 92 shows the presence of TRAPS in porcine skin over a period of 21 days when a sensor has been implanted. As shown by the results, there are numerous TRAPs early on, i.e. at day 1. The significant number of TRAPS impedes sensor performance due to tissue inflammation and fibrosis. At day 7, TRAPS are still present, and then start to disappear as can be seen by comparing day 7 to day 14. However, during this time, fibrosis appears and “kills” the tissue.

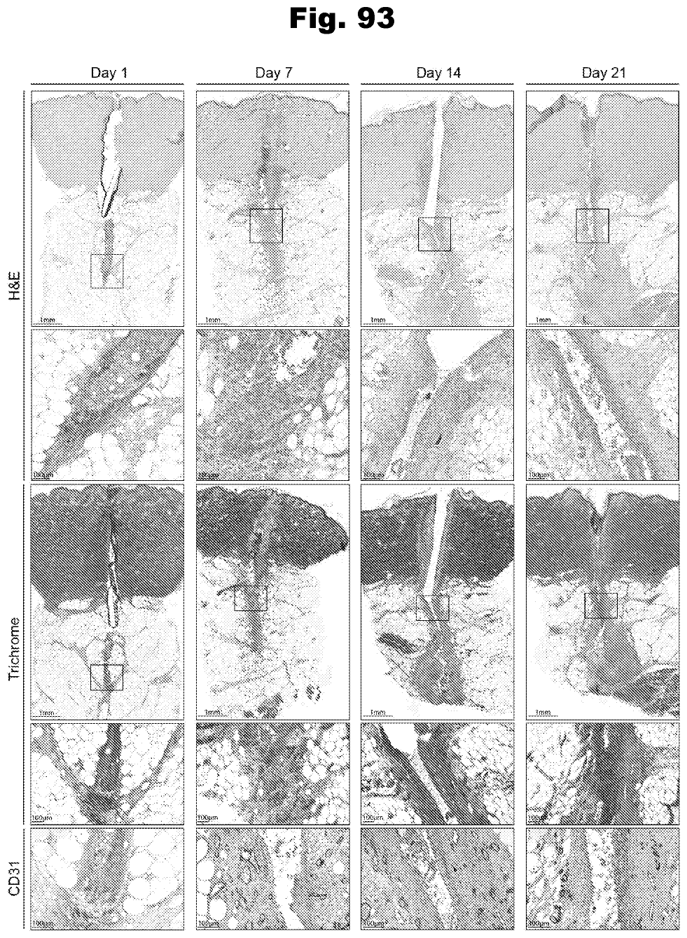

FIG. 93 shows H&E, Trichrome and CD31 (vessel staining) for the porcine skin, for the samples tested in connection with FIG. 92. This figure shows the sensor-induced inflammation and scarring/fibrosis at the site of sensor implantation. This figure also shows the association of the TRAPs with the tissue inflammation at the sensor implantation site.

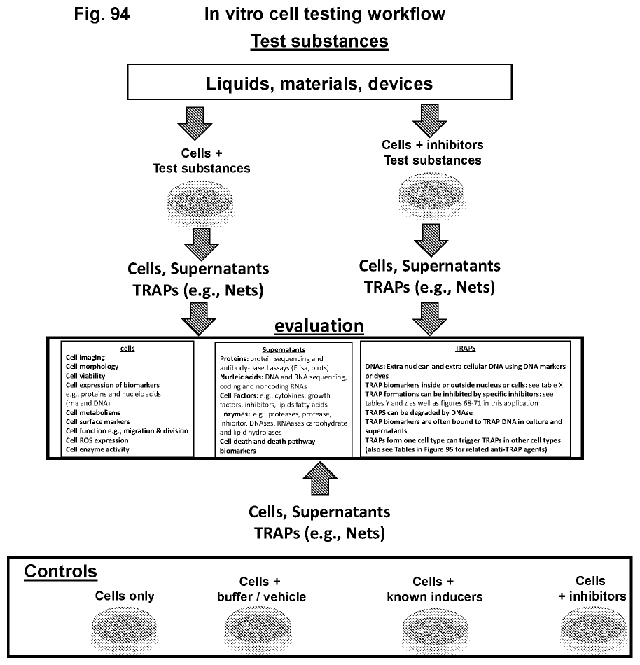

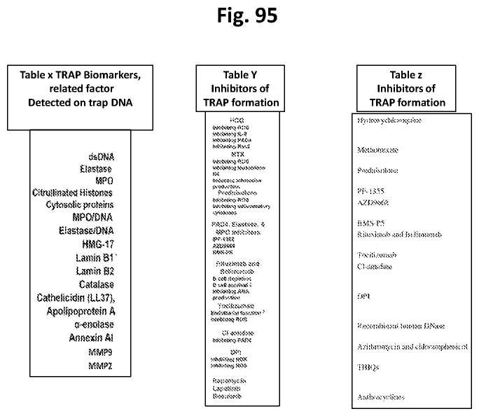

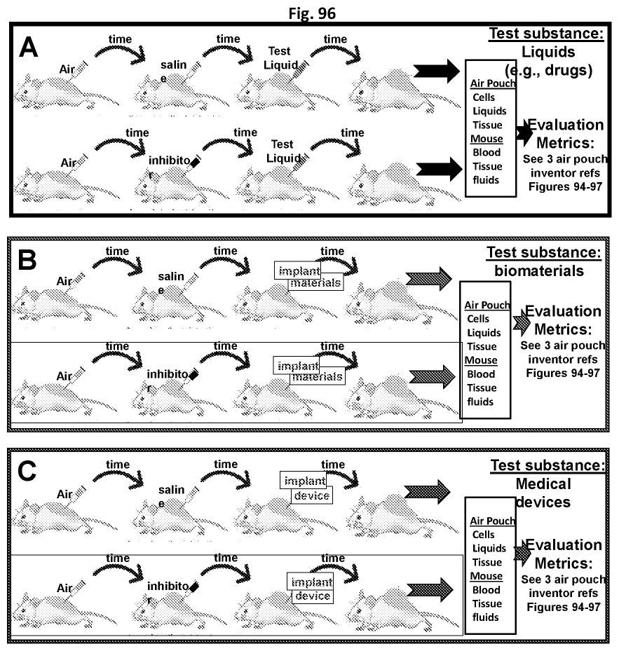

FIG. 94 is a diagram of a workflow that can be used to evaluate liquids, biomaterials, implantable medical devices for their ability to trigger TRAPs from cells in vitro. The workflow also can be used to determine the impact of anti-TRAP agents (FIG. 95) to affect TRAP formation and toxicity in vitro. The method can be used to 1) trigger TRAPs from various cell populations in vitro (see tables C and D for non-limiting examples of cell types above), and/or 2) determine the impact of anti-TRAP agents) to inhibit, diminish or prevent, remove or neutralize TRAPs formation or toxicity in vitro from any cell or group of cells (see FIG. 95 and Tables C and D for cell types).

FIG. 95 provides a list of anti-TRAP agents that can be used in vitro in the workflow of FIG. 94 or in vivo in the workflow of FIG. 96 to determine the most effective anti-TRAP agents related to particular liquids (e.g. drugs preservatives), biomaterials, implantable devices and/or medical devices, the agents being employed individually or in combinations.

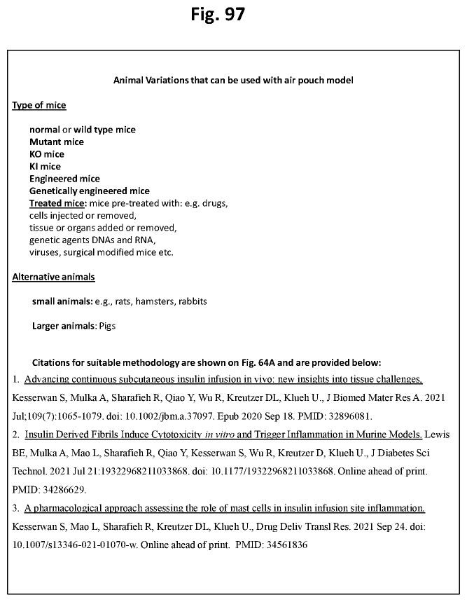

FIG. 96 is a workflow diagram for evaluating the ability of liquids, biomaterials, implantable medical devices to trigger TRAPs in vivo using a mammal (e.g. murine) air pouch model. This workflow also can be used to determine the impact of anti-TRAP agents (FIG. 95) on TRAP formation and TRAP in vivo toxicity in the mammal (mouse) air pouch model. FIG. 96 includes non-limiting examples of methods, routes and timing for testing liquids, biomaterials, implantable medical devices. Further details about using the air pouch model to evaluate cell and tissue toxicity for injuries induced by toxic agent can be found in the articles co-authored by the inventors that are cited in FIG. 97.

FIG. 97 provides a list of alternative animals that can be used for the air pouch model to evaluate the ability of liquids, biomaterials, implantable medical devices for their ability to trigger TRAPs. FIG. 97 also cites four air pouch references co-authored by the inventors that provide further details for executing and evaluating toxicity of substances using the mammal air pouch model in vivo.

### Prophetic Examples of Mammal Air Pouch In Vivo

### PROPHETIC EXAMPLES 1-14

### Example 1—Hydroxychloroquine

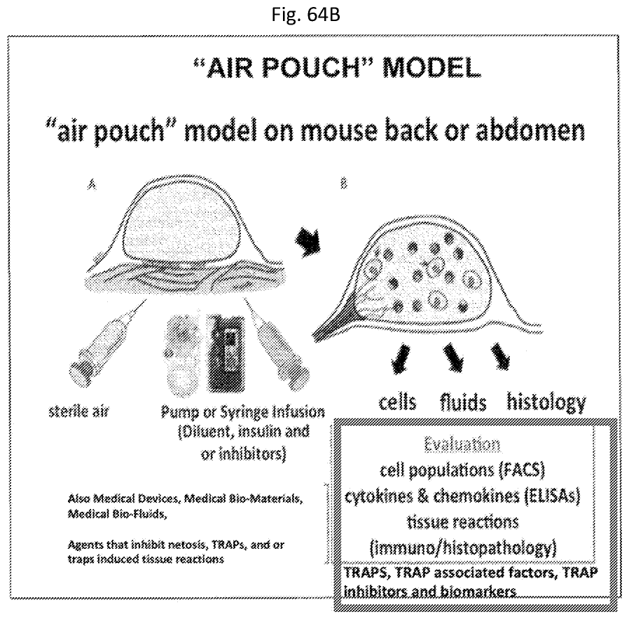

An airpouch is formed in a mouse using the process illustrated and described in association with FIGS. 64A, 64B, 89, 90, 94-97 as well as the inventor's recent publications that are cited above. Hydroxychloroquine is injected into the mouse muscle or introduced into the pouch and the effect on the tissue surrounding the air pouch is examined. It is expected that the hydroxychloroquine inhibits TRAP formation, especially NET formation, using the metrics in the above figures and the inventor's recent publications, the contents of which are incorporated by reference herein in their entirety. One can evaluate the entire mouse systemically after the introduction of the substance into the pouch by sampling blood, urine, other fluids and other tissues.

### Example 2: Using Methotrexate to Inhibit ROS/Adenosine Production

The procedure of Prophetic Example 1 is repeated except that methotrexate is used instead of hydroxychloroquine. It is expected that the methotrexate inhibits TRAP formation, especially NET formation.

### Example 3: Using Prednisolone to Inhibit Production of ROS and Inflammatory Mediators

The procedure of Prophetic Example 1 is repeated except that prednisolone is used instead of hydroxychloroquine. It is expected that the drug inhibits TRAP formation, especially NET formation.

### Example 4: Using PF-1355 to Inhibit MPO

The procedure of Prophetic Example 1 is repeated except that PF-1355 is used instead of hydroxychloroquine. It is expected that the drug inhibits TRAP formation, especially NET formation.

### Example 5. Using AZD9668 to Inhibit Neutrophil Elastase/IL-10, IL-6, IL-8, TNFα

The procedure of Prophetic Example 1 is repeated except that AZD9668 is used instead of hydroxychloroquine. It is expected that the drug inhibits TRAP formation, especially NET formation.

### Example 6: Using BMS-P5 to Inhibit PAD

The procedure of Prophetic Example 1 is repeated except that BMS-P5 is used instead of hydroxychloroquine. It is expected that the drug inhibits TRAP formation, especially NET formation.

### Example 7: Using Prednisolone to Inhibit Anti-CD20 and BlyS

The procedure of Prophetic Example 1 is repeated except that prednisolone is used instead of hydroxychloroquine. It is expected that the drug inhibits TRAP formation, especially NET formation.

### Example 8: Using Tocilizumab to Inhibit Anti-IL-6R

The procedure of Prophetic Example 1 is repeated except that Tocilizumab is used instead of hydroxychloroquine. It is expected that the drug inhibits TRAP formation, especially NET formation.

### Example 9: Using Cl-Amidine to Inhibit PAD4

The procedure of Prophetic Example 1 is repeated except that Cl-amidine is used instead of hydroxychloroquine. It is expected that the drug inhibits TRAP formation, especially NET formation.

### Example 10: Using DPI to Inhibit Gluconeogenesis and Cellular Respiration Enzymes, and Inhibit ROS Production

The procedure of Prophetic Example 1 is repeated except that DPI is used instead of hydroxychloroquine. It is expected that the drug inhibits TRAP formation, especially NET formation.

### Example 11: Recombinant Human DNase

The procedure of Prophetic Example 1 is repeated except that Recombinant human DNase is used instead of hydroxychloroquine. It is expected that the drug decreases tissue reactions by neutralizing TRAPs, especially NETs.

### Example 12: Azithromycin and Chloramphenicol

The procedure of Prophetic Example 1 is repeated except that Azithromycin and chloramphenicol are used instead of hydroxychloroquine. It is expected that the drug combination inhibits TRAP formation, especially NET formation.

### Example 13: THIQs

The procedure of Prophetic Example 1 is repeated except that THIQs is used instead of hydroxychloroquine. It is expected that the drug inhibits TRAP formation, especially NET formation.

### Example 14: Anthracycline

The procedure of Prophetic Example 1 is repeated except that an anthracycline is used instead of hydroxychloroquine. It is expected that the drug inhibits TRAP formation, especially NET formation.

Other embodiments not specifically described also fall within the scope of the appended claims.

1. An implantable device coated with an anti-TRAP agent.

2. The implantable device of claim 1, wherein the anti-TRAP agent comprises an anti-NET agent.

3. The implantable device of claim 1, wherein the agent comprises an inhibitor of at least one member selected from the group consisting of azurocidin, calprotectin, cathelicidins, cathepsin G, defensins, elastase (NE), gelatinase, genomic DNA, histones, lactoferrin, leukocyte proteinase (PR3), lysozyme C, mitochondrial DNA and myeloperoxidase (MPO).

4. The implantable device of claim 1, wherein the anti-TRAP agent comprises at least one of an elastase inhibitor, a protease inhibitor, a citrullination inhibitor, a reactive oxygen species (ROS) inhibitor, a myeloperoxidase (MPO) inhibitor, a NADPH oxidase inhibitor and a toll-like receptor inhibitor.

5. (canceled)

6. The implantable device of claim 1, wherein the agent comprises an intracellular inhibitor.

7. The implantable device of claim 1, wherein the agent comprises an extracellular inhibitor

8. The implantable device of claim 1, wherein the anti-TRAP agent comprises a therapeutic nucleic acid such as coding or noncoding RNA e.g. modified-messenger RNA and/or micro-RNA.

9. The implantable device of claim 1, wherein an antimicrobial agent is combined with the anti-TRAP agent.

10. The implantable device of claim 1, wherein the anti-TRAP agent is contained in a pharmaceutically compatible carrier.

11. The implantable device of claim 10, wherein the carrier comprises at least one of a biological matrix, a synthetic matrix, a nanoparticle, and a liposome.

12. The implantable device of claim 1, wherein the implantable device comprises a sensor, a cannula, a collar for a cannula, a collar for a sensor, a catheter, a suture or a mesh.

13. The implantable device of claim 1, wherein the device is further coated with an anti-inflammatory agent.

14. The implantable device of claim 1, wherein the device comprises a drug-injecting pen.

15. The implantable device of claim 1, wherein the device comprises an infusion set.

16. A method of forming the coated implantable device of claim 1.

17. A kit comprising the coated implantable device of claim 1 enclosed in sterile packaging.

18. An analyte sensor having a sensing element and a support element, at least one of the sensing element and a support being coated with an inhibitor of TRAPS and/or a material that mitigates TRAPs after formation.

19. A method comprising:
obtaining an anti-TRAP agent,
obtaining an implantable device,
coating the implantable device with the anti-TRAP agent, and
implanting the device.

20. The method of claim 19, wherein the implantable device is coated prior to implantation.

21. The method of claim 19, wherein the implantable device is coated after implantation by injecting the inhibitor into tissue surrounding the implantable device.

22-50. (canceled)
